(19) United States
(12) Patent Application Publication
(10) Pub. No.: US 2022/0248988 A1
(43) Pub. Date: Aug. 11, 2022

(54) DIGITAL AND USER INTERFACES FOR ANALYTE MONITORING SYSTEMS

(71) Applicant: ABBOTT DIABETES CARE INC., Alameda, CA (US)

(72) Inventors: Panganamala Ashwin Kumar, Oakland, CA (US); Jennifer Woo, Oakland, CA (US); Stephen A. Rossi, Clayton, CA (US); Sujit Jangam, San Mateo, CA (US); Kendall Covington, Oakland, CA (US); Jordan Wing-Haye Lang, San Jose, CA (US); Andrew Revoltar, Burien, WA (US); William Koo Lee, Alameda, CA (US); Kimberly Hilton, San Francisco, CA (US); Jeremy Hurwitz, San Mateo, CA (US); Saranpreet Nagra, Union City, CA (US); Wesley Scott Harper, Alameda, CA (US); Swati Satish, Fremont, CA (US); Gina Correa, San Jose, CA (US); Duncan P. Williams, Pleasanton, CA (US); Naveen Thuramalla, Dublin, CA (US); Andreana Dereniak, Oakland, CA (US); Justin N. Williams, Oakland, CA (US); Marc B. Taub, Mountain View, CA (US); James P. McCarter, St. Louis, MO (US); Monica J. Wilkins, Lake Bluff, IL (US); Namvar Kiaie, Danville, CA (US); Ismene Grohmann, San Francisco, CA (US); Laura C. Brandner, Oakland, CA (US); Michael L. Bird, Bend, OR (US); Steven Stratis, Orlando, FL (US)

(21) Appl. No.: 17/478,447

(22) Filed: Sep. 17, 2021

Related U.S. Application Data
(60) Provisional application No. 63/143,584, filed on Jan. 29, 2021.
(60) Provisional application No. 63/090,204, filed on Oct. 10, 2020.
(60) Provisional application No. 63/080,763, filed on Sep. 20, 2020.
(60) Provisional application No. 63/079,850, filed on Sep. 17, 2020.

(51) Int. Cl. A61B 5/145; A61B 5/00

(52) U.S. Cl. CPC A61B 5/14532; A61B 5/7475; A61B 5/7435; A61B 2562/0271; A61B 5/0022; A61B 2560/0475; A61B 5/746

(57) ABSTRACT

Improved digital interfaces, graphical user interfaces, and alarms for analyte monitoring systems are provided. For example, disclosed herein are various embodiments of methods, systems, and interfaces for signal loss condition determination, Time-in-Ranges interfaces, GMI metrics, urgent low glucose alarms, alarm suppression features, alarm setup interfaces, and alarm unavailability detection features. In addition, various embodiments of interfaces for alarm logging and compatibility checking of an analyte monitoring software application are described. Also, various embodiments of interface enhancements are described, including an enhanced visibility mode, a voice accessibility mode, additional interfaces relating to user privacy, as well as caregiver alarms, among other embodiments.

### CROSS-REFERENCE TO RELATED APPLICATIONS

This application claims priority to U.S. Provisional Application No. 63/143,584, filed Jan. 29, 2021, U.S. Provisional Application No. 63/090,204, filed Oct. 10, 2020, U.S. Provisional Application No. 63/080,763, filed Sep. 20, 2020, and U.S. Provisional Application No. 63/079,850, filed Sep. 17, 2020, all of which are herein expressly incorporated by reference in their entirety for all purposes.

### FIELD

The subject matter described herein relates generally to digital interfaces, user interfaces, and alarms for analyte monitoring systems, as well as systems, methods, and devices relating thereto.

### BACKGROUND

The detection and/or monitoring of analyte levels, such as glucose, ketones, lactate, oxygen, hemoglobin A1C, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with the bodily fluid. The analyte monitoring system may also be configured to transmit analyte data and/or alarms to another device, from which a caregiver such as, for example, a parent, a spouse, or a health care provider (“HCP”), can review the data and make therapy decisions. Furthermore, the benefits of analyte monitoring systems are not limited to persons with diabetes. For instance, analyte monitoring systems can provide useful information and insights to individuals interested in improving their health and wellness. As one example, to improve their sports performance, athletes can utilize a sensor control device worn on the body to collect data relating to one or more analytes such as, for example, glucose and/or lactate. Other non-medical applications for analyte monitoring systems are possible and described in further detail below.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Furthermore, as sensor control devices have become more convenient, comfortable, and affordable for users, applications outside of medicine have become feasible. For example, high-performance athletes are interested in optimizing levels of performance-affecting analytes, for example blood glucose, before or during training and competition. However, some existing user interfaces for sensor control devices are designed for medical use by patients under care of a physician, and not for non-medical applications such as, for example, athletic training and competition. As such, the data collected by the sensor control device, and methods for presenting the data to the user, may be unsuitable for non-medical applications. In addition, sensor control devices for non-medical (e.g., wellness and fitness) use may be confused with similar devices made for medical use, leading to problems in interpreting or using data.

Thus, needs exist for digital interfaces, graphical user interfaces, and alarms for analyte monitoring systems for medical and/or non-medical use, as well as methods and devices relating thereto, that are robust, user-friendly, and provide for timely and actionable responses.

### SUMMARY

Provided herein are example embodiments of digital and user interfaces for analyte monitoring systems. Aspects of the inventions are set out in the independent claims and preferred features are set out in the dependent claims. Preferred features of each aspect may be provided in combination with each other within particular embodiments and may also be provided in combination with other aspects. According to some embodiments, methods, systems, and interfaces relating to determining a signal loss condition in an analyte monitoring system based on a time elapsed since a last current sensor reading are described. In other embodiments, methods, systems, and interfaces for determining an invalid current sensor reading in an analyte monitoring system are described. In still other embodiments, methods, systems, and interfaces relating to determining a “no recent valid sensor reading” alarm condition in an analyte monitoring system are also described.

According to another embodiment, methods for calculating percentages of time relating to an analyte level range or threshold for generating a Time-in-Ranges interface are described. In certain embodiments, the calculated percentages of time can include non-configurable and user-configurable analyte level ranges and/or thresholds.

According to another embodiment, an enhanced visibility mode is provided for an analyte monitoring system software application, in which numerous interfaces for use with an analyte monitoring system can be modified for better visibility in a low light environment. In some embodiments, the enhanced visibility mode can be enabled manually by the user through the operating system of the device. In other embodiments, the enhanced visibility mode can be enabled by a light sensor or according to a predetermined schedule.

According to another embodiment, a voice accessibility mode is provided for an analyte monitoring system software application, in which audible output of interfaces (or portions thereof) for an analyte monitoring system can be generated. In some embodiments, for example, the voice accessibility mode can convert touched portion of a display into an audible output. In other embodiments, certain touch responsive portions of an interface can be configured in groupings such that a device can convert text of an entire grouping into audible output in response to the user touching any portion associated with the grouping.

According to other embodiments, additional embodiments of digital and user interfaces for analyte monitoring are provided. In some embodiments, for example, a sensor usage report interface is provided in which a “view” metric is generated based on the number of instances in which a user views a sensor results interface with a valid sensor reading. In other embodiments, interfaces for an analyte monitoring software application are provided to allow for an “accountless mode,” in which a user need not create or sign into a cloud-based server in order to utilize the analyte monitoring software application. In other embodiments, interfaces for an analyte monitoring software application are provided to allow a user to opt-in or decline to share the user's sensor readings and/or other product-related data for research purposes. In still other embodiments, an interface for an analyte monitoring software application is provided to warn a user about possible false high sensor readings as a result of daily use of Vitamin C supplements above 500 mg.

According to another embodiment, methods, systems, and interfaces are provided for generating alarms relating to an analyte monitoring system on a caregiver's reader device. In some embodiments, for example, a sensor control device worn on a patient's body can wirelessly communicate current sensor readings to the patient's reader device which, in turn, can wirelessly communicate the current sensor readings to a cloud-based server. According to an aspect of the embodiments, the cloud-based server can determine whether the received current sensor readings satisfy an alarm condition and, if so, transmit an alarm indicator to the caregiver's reader device. In other embodiments, for example, an alarm condition is determined on the patient's reader device and, in response to the detection of the alarm condition, an alarm indicator is transmitted to the cloud-based server and subsequently “passed through” to the caregiver's reader device.

According to some embodiments, methods, systems, and interfaces relating to displaying a user interface including at least one glucose management indicator metric (GMI) determined for a time period are described.

According to some embodiments, methods, systems, and interfaces relating to displaying a user interface including at least one GMI metric determined for a time period, a glucose trend interface, a sensor user interface comprising a percentage time sensor active metric; and a health information interface is described.

According to some embodiments, methods, systems, and interfaces relating to displaying a user interface including at least one GMI metric determined for a time period, a plot of glucose data measurements taken from the subject across a horizontal representation of a plurality of time segments of a day; and a table comprising a column for each of the plurality of different time segments of the day, each column including both the assessment of the risk of hypoglycemia and the assessment of glycemic variability for one of the plurality of different time segments of the day.

According to some embodiments, methods, systems, and interfaces relating to displaying a user interface including a glucose statistics interface comprising at least one glucose management indicator (GMI) metric determined for a time period; a time in ranges interface; a plot of glucose readings over the time period, wherein the plot displays a median glucose trace, and a plurality of traces for glucose readings at different percentiles; and a plurality of glucose profiles comprising a glucose profile for each day of the time period.

In some embodiments, the at least one GMI metric may be a GMI percentage. In some embodiments, the at least one GMI metric may be a GMI value in mmol/mol. In some embodiments, the at least one GMI metric may be two GMI metrics. In some embodiments, both the GMI percentage and the GMI value in mmol/mol may be displayed.

In some embodiments, systems, methods, and interfaces for urgent low glucose alarms in an analyte monitoring system are provided, wherein the analyte monitoring system comprises a sensor control device configured to collect data indicative of an analyte level in a subject. The analyte monitoring system further comprises a reader device (e.g., smart phone) having wireless communication circuitry, and one or more processors coupled with a memory storing instructions that, when executed by the one or more processors, cause the processors to determine whether the data indicative of the analyte level meets one or more alarm conditions. The one or more alarm conditions comprise a first alarm condition associated with a first set of alarm settings that are configurable by a user, and a second alarm condition associated with a second set of alarm settings that are not configurable by the user, wherein the second alarm condition is an urgent low glucose alarm condition. In some embodiments, the second set of alarm settings can include, for example, a non-configurable on-off setting, a non-configurable urgent low threshold setting, a non-configurable alarm tone setting, and/or a non-configurable setting to override a “Do Not Disturb” feature.

According to other example embodiments, methods and systems for alarm suppression are provided. In some embodiments, for example, systems and methods for suppressing alarms during a post-alarm presentation period are described. In particular, a reader device (e.g., a smart phone) comprises one or more processors coupled with a memory, the memory storing instructions that, when executed by the one or more processors, cause the one or more processors to present a first alarm associated with a first condition, receive data indicative of an analyte level from a sensor control device, and determine if a second alarm condition is present based on either the received data indicative of the analyte level or a lack thereof. If a second alarm condition is present, and the second alarm condition is the same as the first alarm condition, then a determination is made as to whether a post-alarm presentation period has elapsed. If the post-alarm presentation period has not elapsed, then no action is taken with respect to the first alarm. If the post-alarm presentation period has elapsed, then the first alarm is either updated or cleared.

As another example, in some embodiments, systems and methods for suppressing alarms during an active dismiss period, which is a predetermined period of time after a user dismisses an alarm presentation, are described. In particular, a reader device (e.g., a smart phone) comprises one or more processors coupled with a memory, the memory storing instructions that, when executed by the one or more processors, cause the one or more processors to present a first alarm associated with a first alarm condition, begin an active dismiss period in response to receiving an indication that the first alarm is dismissed, receive data indicative of an analyte level from a sensor control device, and determine if a second alarm condition is present based on either the received data indicative of the analyte level or a lack thereof. If a second alarm condition is present, and the second alarm condition is the same as the first alarm condition, then a determination is made as to whether an active dismiss period has elapsed or is canceled. If the active dismiss period has either elapsed or is canceled, then a second alarm associated with the first alarm condition is presented. If the active dismiss period has not elapsed and is not canceled, then no further action is taken.

According to other embodiments, alarm setup interfaces for use with an analyte monitoring software application are also described. In some embodiments, for example, the alarm setup interfaces can include one or more interfaces for configuring alarm permission settings, such as a notification permission setting, a critical alerts permission setting, a location permission setting, a battery optimization setting, or a “Do Not Disturb” setting. According to one aspect of these embodiments, the analyte monitoring software application can be configured to remain in an inoperable state or a partially operable state unless the one or more alarm permission settings are enabled.

According to some embodiments, systems and methods for detecting alarm unavailability conditions are described. In particular, a reader device (e.g., a smart phone) comprises one or more processors coupled with a memory, the memory storing instructions that, when executed by the one or more processors, cause the one or more processors to detect one or more alarm unavailability conditions while at least one alarm of the analyte monitoring system is enabled, and present a notification associated with the detected one or more alarm unavailability conditions. In some embodiments, the one or more alarm unavailability conditions can include one or more of: a wireless communication circuitry being disabled or malfunctioning, one or more systemwide notifications being disabled, one or more application-specific notifications being disabled, a forced closure of an analyte monitoring software application, one or more critical alerts being disabled, an override “Do Not Disturb” feature being disabled, one or more alarm tones being set to silent, location permissions being disabled, one or more battery optimization features being enabled, no active sensor detected, or a sensor fault condition.

According to some embodiments, systems and interfaces for logging alarms in an analyte monitoring system are also described. In still other embodiments, methods and systems for determining the compatibility of an analyte monitoring software application are described.

According to some embodiments, systems, devices and methods for a sensor user interface in an in vivo analyte monitoring system adapted for non-medical use are also described.

According to some embodiments, provided herein are improvements to a user interface to make a sensor control device useful for non-medical applications. Other improvements and advantages are provided as well. The various configurations of these devices are described in detail by way of the embodiments which are only examples.

In some embodiments, for example, a method for an electronic interface of a computing device may include receiving, by at least one processor, sensor data from the sensor control device over a predetermined period of time, and determining whether the measurement value of the sensor data satisfies at least one necessary condition for providing such non-medical information to a display device. The method may further include providing, to a display device, an interactive graphical user interface configured for display of the sensor data based on the determining, wherein the display device only indicates one or more measurement values of the sensor data that satisfy the at least one condition.

In illustrative embodiments, the method includes using the at least one necessary condition that includes at least one of a predetermined upper threshold and lower threshold for the measurement values, wherein the thresholds are set to be indicative of a medical pathology or condition. For example, the method may include providing out-of-range values falling outside the predetermined threshold range to the interface without indicating a specific value to the display device. Additionally, when measurement values satisfy at least one condition for display as non-medical information, the method may include indicating specific values on the display device in text form. In one embodiment, for example, the method may include providing the sensor data from the sensor control device that indicates glucose level for a user wearing a control device on a display device, wherein the predetermined lower and upper measurement threshold values are 55 and 200 mg/dL, respectively. The method may include determining, by the at least one processor, that measurement values falling within this glucose range satisfy the at least one condition for display and therefore providing the measurement value for display by a display device, whereas a value falling outside this range triggers the out-of-range indicator. Conversely, the method may further include indicating that the measurement values are out of range without providing an exact measurement value to the reader device. In an aspect, the method may include providing a graphical user interface with a graph of the sensor data over time. The method may include configuring the graph with other information, for example, an indicator of a target average value for the sensor data. In an aspect, the graph may omit display of data that is out of range.

In another aspect, a sensor control device for non-medical (e.g., wellness and fitness) use is configured so that it cannot be accessed by a reader device or application that is configured for use with medical sensor control devices. Likewise, the reader device or application for non-medical use is configured so that it cannot access data transmitted by medical sensor control devices.

The reader device, which receives sensor data from the sensor control device, can be a smart phone, tablet computer, personal digital assistant or other proprietary or non-proprietary mobile computing platform. One or more applications can be installed on the reader device, which analyzes data transmitted from the sensor control device and displays non-medical information relating to wellness and nutrition to the individual. Accordingly, the reader device includes at least one data processing unit coupled to a computer memory and to a wireless interface for receiving data and determining whether the measurement value of the sensor data satisfies at least one necessary condition for display as non-medical information. In some embodiments, the memory may hold instructions for limiting display of the measurement values form the sensor control device within a restricted range that is not indicative of a medical pathology, and related operations or aspects as summarized above and/or described in the following detailed description.

Many of the embodiments provided herein are improved GUIs or GUI features for analyte monitoring systems that are highly intuitive, user-friendly, and provide for rapid access to physiological information of a user. More specifically, these embodiments allow a user to easily navigate through and between different user interfaces that can quickly indicate to the user various physiological conditions and/or actionable responses, without requiring the user (or an HCP) to go through the arduous task of examining large volumes of analyte data. Furthermore, some of the GUIs and GUI features and interfaces allow for users (and their caregivers) to better understand and improve their respective levels of engagement with their analyte monitoring systems, and/or to troubleshoot complex system settings to ensure that alarms are functioning properly within an analyte monitoring system. Likewise, many other embodiments provided herein comprise improved digital interfaces, methods, and/or features for analyte monitoring systems that improve upon a caregiver's ability to determine an adverse condition of the patient. Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. Where a method is described and claimed herein, analyte monitoring systems comprising means for performing each of the steps of the method are also expressly disclosed and provided. Moreover, computer programs, computer program products and computer readable media for implementing the steps of the method are also disclosed and provided. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIGS. 3A to 3D are flow diagrams depicting example embodiments of methods for signal loss detection.

FIGS. 4A to 4E are example embodiments of GUIs relating to signal loss conditions and signal loss alarms.

FIGS. 4F to 4H are example embodiments of GUIs comprising configuration interfaces for signal loss alarms.

FIGS. 4I and 4J are example embodiments of GUIs comprising signal loss alarms.

FIG. 5A to 5C are flow diagrams depicting example embodiments of methods for generating graphical representations of Time in Ranges interfaces.

FIG. 6 is a flow diagram depicting an example embodiment of a method for enabling an enhanced visibility mode.

FIGS. 7A-1 to 7I-2 are example embodiment of GUIs shown in normal mode and enhanced visibility mode.

FIG. 8 is a flow diagram depicting an example embodiment of a method for enabling a voice accessibility mode.

FIGS. 9A and 9B are example embodiments of GUIs for use with a voice accessibility mode.

FIG. 10A is an example embodiment of a GUI for a sensor usage report.

FIGS. 10B and 10C are example embodiments of GUIs relating to GMI.

FIG. 10D is an example embodiment of a GUI for snapshot reports including GMI.

FIGS. 10E and 10F are example embodiments of GUIs for insight reports including GMI.

FIG. 10G is an example embodiment of a GUI for glucose profile reports including GMI.

FIGS. 11A to 11D are example embodiments of additional GUIs relating to an analyte monitoring software application.

FIGS. 12A and 12B are further example embodiments of additional GUIs relating to an analyte monitoring software application.

FIG. 13A is a flow diagram depicting an example embodiment of a method for determining and presenting alarms in an analyte monitoring system.

FIGS. 13B to 13G are example embodiments of GUIs relating to various alarms in an analyte monitoring system.

FIG. 14A to 14I are example embodiments of GUIs relating to various alarm settings in an analyte monitoring system.

FIGS. 15A and 15B are flow diagrams depicting example embodiments of methods for suppressing alarms in an analyte monitoring system.

FIG. 15C is a diagram depicting an example embodiment for an alarming scheme.

FIGS. 16A to 16E are example embodiments of GUIs relating to the configuration of alarms in an analyte monitoring system.

FIGS. 17A to 17I are also example embodiments of GUIs relating to the configuration of alarms in an analyte monitoring system.

FIG. 18A is a flow diagram depicting an example embodiment of a method for the determination and notification of various alarm unavailable conditions.

FIGS. 18B and 18C are example embodiments of GUIs relating to various alarm unavailability conditions in an analyte monitoring system.

FIGS. 18D to 18N are example embodiments of modals relating to various alarm unavailability conditions in an analyte monitoring system.

FIGS. 18O to 18Q are example embodiments of GUIs relating to various alarm unavailability conditions in an analyte monitoring system.

FIG. 19 is a flow diagram depicting an example embodiment of a method for logging alarms in an analyte monitoring system.

FIGS. 20A to 20C are example embodiments of GUIs relating to alarm logging in an analyte monitoring system.

FIGS. 21A to 21J are example embodiments of GUIs relating to compatibility checking in an analyte monitoring system.

FIG. 22 is a system overview of an analyte monitoring system comprising a sensor control device, a patient reader device, a network, a trusted computer system, and a caregiver reader device.

FIGS. 23A and 23B are flow diagram depicting example embodiments of methods for providing caregiver alarms.

FIGS. 24A to 24H are example embodiment of GUIs relating to a caregiver application and alarm configuration settings included therewith.

FIG. 25 is a flowchart illustrating a process for an electronic interface of a computing device that displays non-medical data from a sensor control device.

FIG. 26 is a screenshot illustrating an example of a user interface provided by a method as described herein.

FIGS. 27-28 are flowcharts further illustrating a process for an electronic interface of a computing device that displays non-medical data from a sensor control device.

FIG. 29A to 29F are example embodiments of GUIs for displaying non-medical data from a sensor control device on a wearable device.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include GUIs, alarms, and digital interfaces for analyte monitoring systems, and systems, methods, and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

As previously described, a number of embodiments described herein provide for improved GUIs, alarms, and digital interfaces for analyte monitoring systems, wherein the alarms and GUIs are actionable, user-friendly, and provide for rapid access to physiological information of a user. According to some embodiments, for examples, methods and interfaces are provided for determining a signal loss condition, an invalid current sensor reading condition, or a “no recent valid sensor reading” condition in an analyte monitoring system. According to other embodiments, methods and systems are provided for generating Time-in-Ranges interfaces based on configurable and non-configurable analyte level ranges and thresholds. According to still other embodiments, methods, systems, and interfaces relating to an enhanced visibility mode and a voice accessibility mode are provided to improve the visual and auditory accessibility of an analyte monitoring software application. According to some embodiments, for example, methods and interfaces are provided for determining an alarm unavailability condition in an analyte monitoring system. According to other embodiments, methods and systems are provided for determining the compatibility of an analyte monitoring software application. Additional improved digital and user interfaces for an analyte monitoring software application are described.

A number of embodiments of systems, devices, and methods are described herein that provide for monitoring and managing the wellness and nutrition of an individual based at least in part on analyte data from an in vivo analyte sensor. For example, several embodiments of the present disclosure are designed to enable a user to track and understand of performance-affecting analytes, for example blood glucose, over time, before, during, and after training or athletic performances, using a commonly available sensor control device. Thus informed, athletes and their trainers can better understand the efficacy of their nutrition choices as it pertains to athletic conditioning and performance. For example, a user may monitor glucose levels using a sensor control device and reader device, thereby allowing an individual to correlate low glucose levels with performance-hindering results, such as fatigue. Additionally, the availability of glucose data measured at frequent intervals over time may inform the user when nutrients are needed to replenish and help achieve peak athletic performance. In other embodiments, monitoring biosensors at non-medical levels ensures the user that analyte levels are maintained in a target range. For example, for sports purposes, an athlete may maintain analyte levels within a range, such as between 55 and 200 mg/dL. Advantageously, only glucose levels within this range are displayed with a specific value. Consequently, these embodiments can provide non-medical analyte data to users to help promote wellness and athletic performance, among other advantages.

According to another embodiment, methods, systems, and interfaces are provided for generating alarms relating to an analyte monitoring system on a caregiver's reader device. In some embodiments, for example, a sensor control device worn on a patient's body can wirelessly communicate current sensor readings to the patient's reader device which, in turn, can wirelessly communicate the current sensor readings to a cloud-based server. According to an aspect of the embodiments, the cloud-based server can determine whether the received current sensor readings satisfy an alarm condition and, if so, transmit an alarm indicator to the caregiver's reader device. In other embodiments, for example, an alarm condition is determined on the patient's reader device and, in response to the detection of an alarm condition, an alarm indicator is transmitted to the cloud-based server and subsequently “passed through” to the caregiver's reader device.

Collectively and individually, these methods, systems, and digital and user interfaces improve upon the accuracy, integrity, and privacy of analyte data being collected by an analyte monitoring system, the flexibility of the analyte monitoring system by allowing caregivers to receive information about a patient's condition, and the alarming capabilities of the analyte monitoring system by providing for more robust signal loss detection, to name only a few. These methods, systems, and digital and user interfaces also improve upon the convenience, practicality, and utility of analyte monitoring system by allowing people suffering from diabetes to have access regularly to a valuable glucose metric (GMI) that will help them manage their diabetes. In the past, patients would only learn their A1C level by getting a doctor's order for a blood test. The user interfaces described herein take advantage of the voluminous data received from continuous glucose monitors and flash glucose monitors to calculate and display the GMI metric, which is a good indicator of A1C, to the patient at will. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

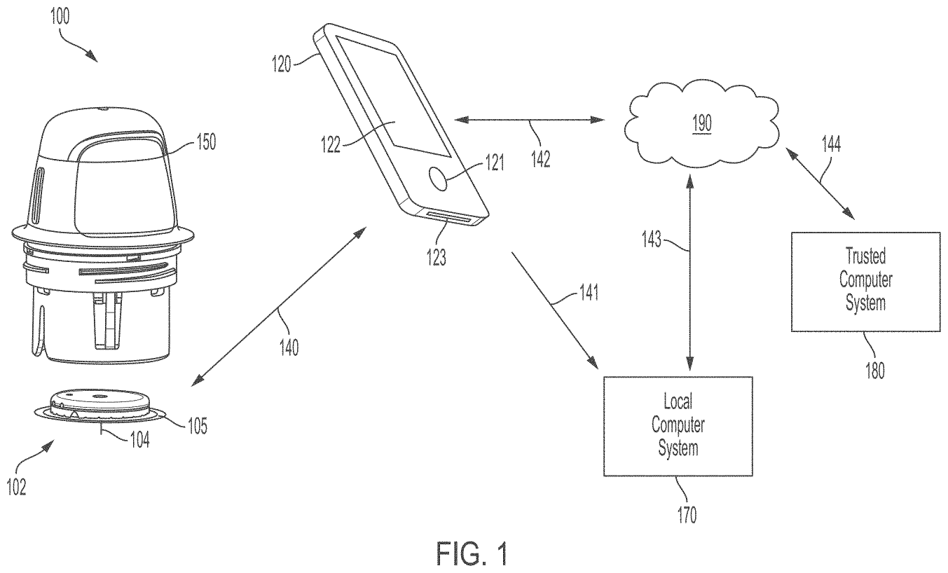

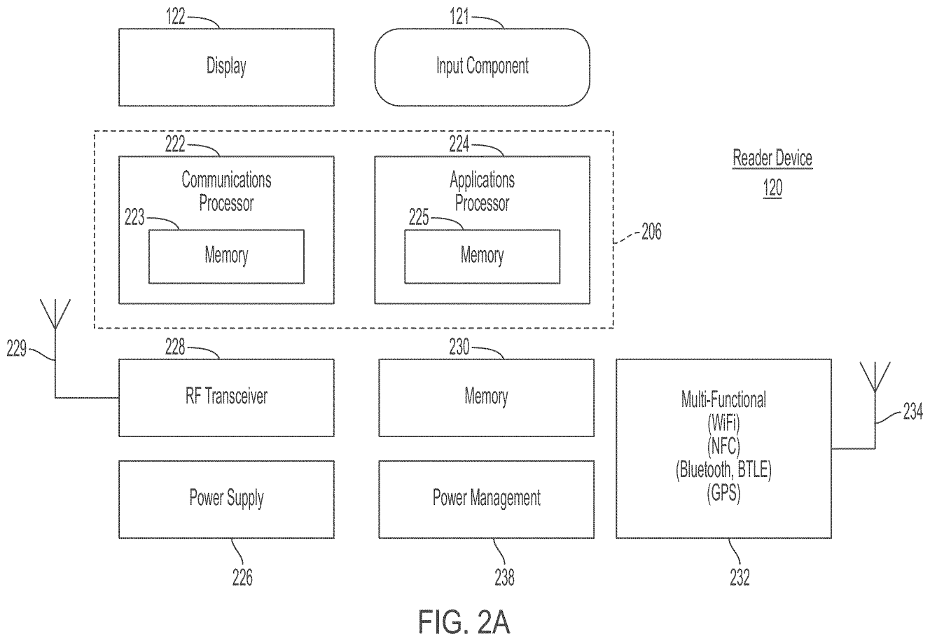

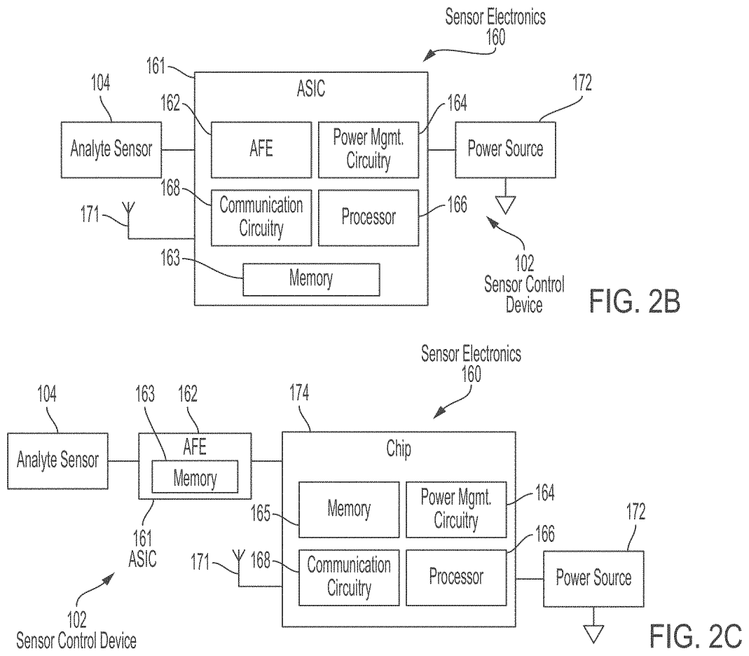

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, data acquired from sensor control device 102 can be stored on reader device 120. According to one aspect of some embodiments, such data can include the model number and serial number of sensor control device 102, as well as information relating to the sensor control device 102′s status, market code, or network address. In some embodiments, such data can also include error events detected by sensor control device 102. In addition, in some embodiments, either or both of current glucose values and historical glucose values can include one or more time stamps (e.g., factory time, UTC time, user's local time based on time zone, and the current time zone).

In some embodiments, sensor control device 102 can store data such that if reader device 120 is not in communication with sensor control device 102 (e.g., if reader device 120 is out of a wireless communication range, is powered off, or is otherwise unable to communicate with sensor control device 102), when reader device 120 re-establishes communication with sensor control device 102, data can then be backfilled to reader device 120. According to some embodiments, data that can be backfilled can include, but is not limited to, current and historical glucose values, as well as error events. Further details regarding data backfilling can be found in U.S. Pat. No. 10,820,842, as well as U.S. Publ. No. 2021/0282672 (“the '672 Publication”), both of which are hereby incorporated by reference in their entireties for all purposes.

According to some embodiments, each current glucose value and/or historical glucose value acquired from sensor control device 102 can further be validated on reader device 120, such as, for example, by performing a CRC integrity check to ensure that the data has been transferred accurately. In some embodiments, for example, a data quality mask of the current glucose value and/or historical glucose value can be checked to ensure that the reading is correct and can be displayed as a valid reading on the reader device 120.

According to another aspect of some embodiments, reader device 120 can include a database for storing any or all of the aforementioned data. In some embodiments, the database can be configured to retain data for a predetermined period of time (e.g., 30 days, 60 days, 90 days, six months, one year, etc.). According to some embodiments, the database can be configured to delete data after it has been uploaded to a cloud server. In other embodiments, database can be configured for a clinical setting, in which data is retained for a longer period of time (e.g., one year) relative to a non-clinical setting. In addition to the aforementioned data (e.g., current and/or historical glucose values, error events, etc.), the database on reader device 120 can also store user configuration information (e.g., login ID, notification settings, regional settings, and other preferences), as well as application configuration information (e.g., cloud settings, URLs for uploading data and/or error events, version information, etc.). The database can be encrypted to prevent a user from inspecting the data content directly even if the operating system of reader device 120 is compromised.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Graphical User Interfaces for Analyte Monitoring Systems

Described herein are example embodiments of GUIs for analyte monitoring systems, as well as methods and system relating thereto. As an initial matter, it will be understood by those of skill in the art that the GUIs and related methods described herein comprise instructions stored in a non-transitory memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the non-transitory memory of a single centralized device or, in the alternative, can be distributed across multiple discrete devices in geographically dispersed locations.

Also described herein are example embodiments of alarms, alarm features, and alarm settings for analyte monitoring systems, as well as methods, systems, and GUIs relating thereto. As an initial matter, it will be understood by those of skill in the art that the alarms, alarm features, and alarm settings, as well as the related methods and interfaces described herein, can comprise instructions (e.g., software, firmware, etc.) stored in non-transitory memory of a sensor control device 102, reader device 120, local computer system 170, trusted computer system 180, and/or any other computing device or system (e.g., cloud-based server) that is part of, or in communication with, analyte monitoring system 100. These instructions, when executed by one or more processors of the corresponding system or device, cause the one or more processors to perform any or all of the method steps, and/or output the alarms or alarm interfaces described herein. Those of skill in the art will further recognize that the alarms, alarm features, alarm settings, and alarm interfaces described herein can be stored as instructions in the non-transitory memory of a single centralized device or, in the alternative, can be distributed across multiple discrete devices in geographically dispersed locations.

### Example Embodiments of Signal Loss Indicators, Alarms, and Other Errors Conditions

Example embodiments of methods and systems for determining a signal loss condition, generating signal loss indicators and alarms, and outputting signal loss interfaces and GUIs will now be described. According to one aspect of the embodiments, a signal loss condition can refer to a state of a device capable of wireless communication in an analyte monitoring system, such as a reader device (e.g., smart phone), sensor control unit, or local computing system, in which the device has not received a current sensor reading for a predetermined period of time. According to another aspect of the embodiments, a signal loss alarm condition can comprise a state in which the device has not received a valid current sensor reading for another predetermined period of time. In some embodiments, an invalid current sensor reading can comprise one of a failure to receive a current sensor reading within a predetermined time period, an error relating to a sensor signal, or an error relating to the temperature of the sensor.

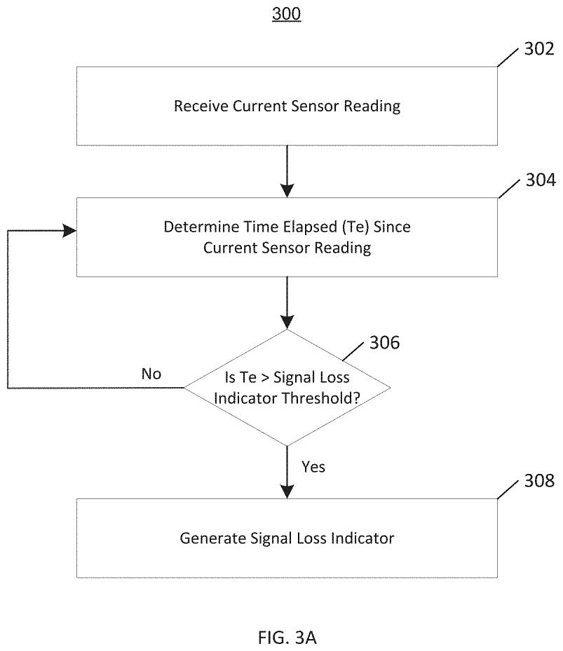

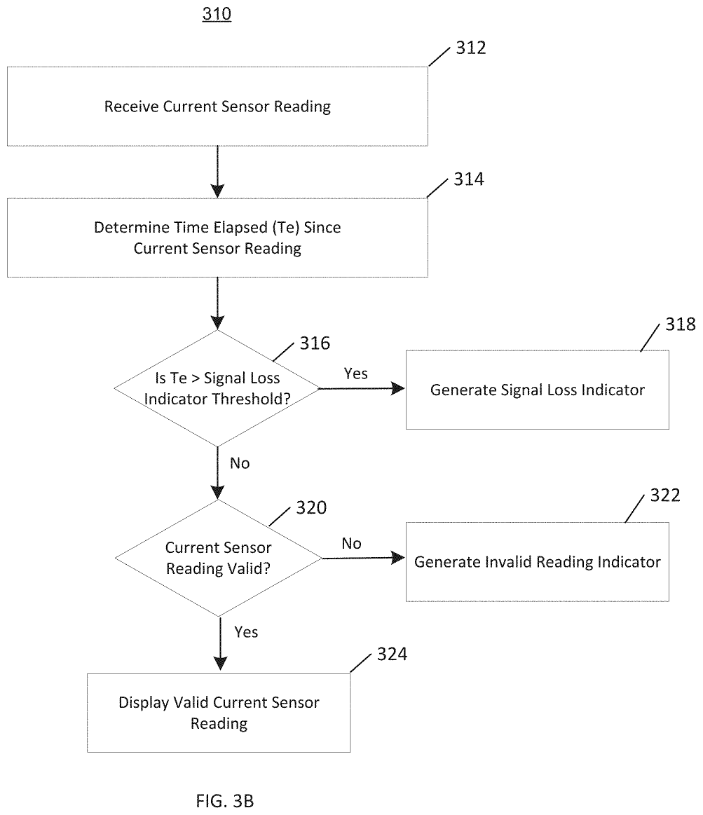

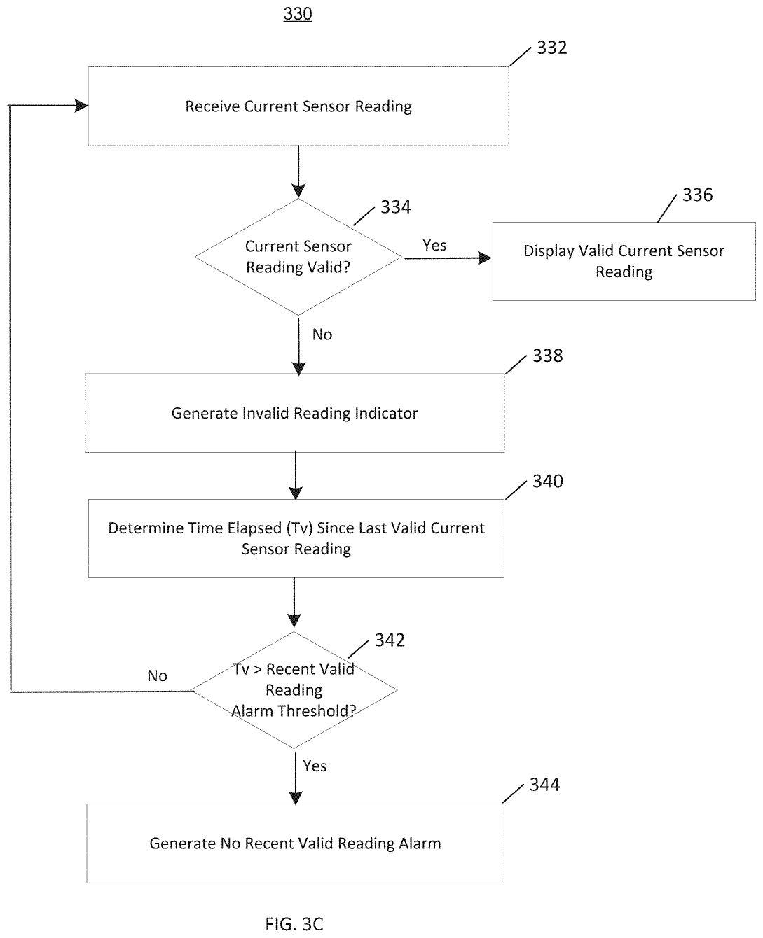

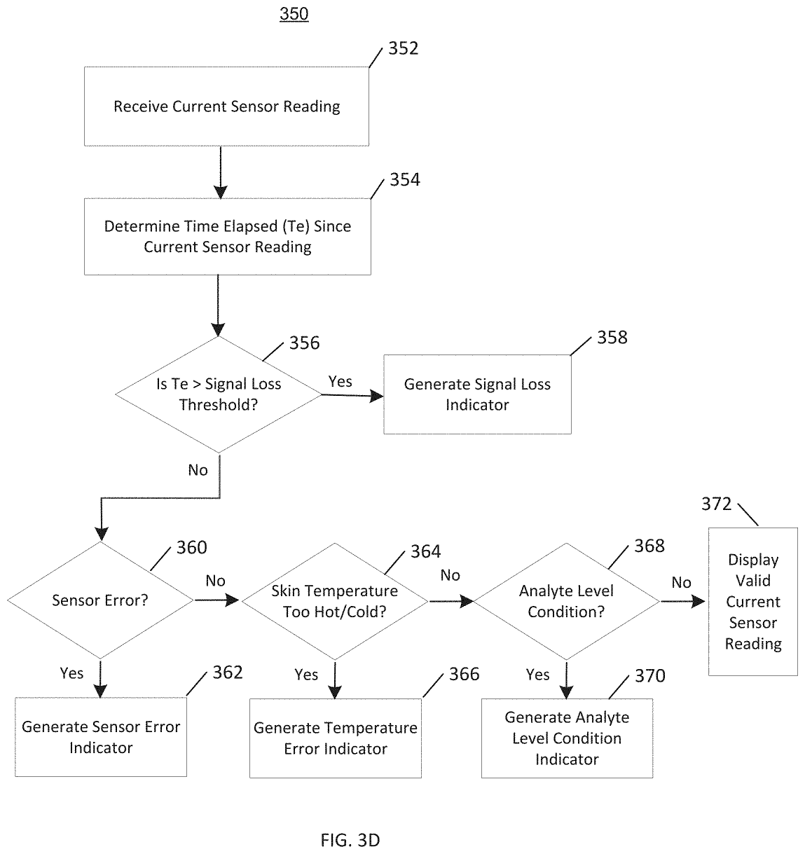

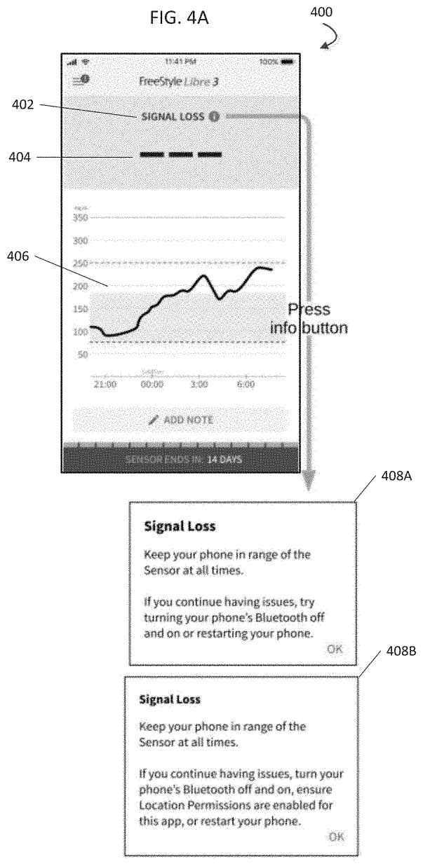

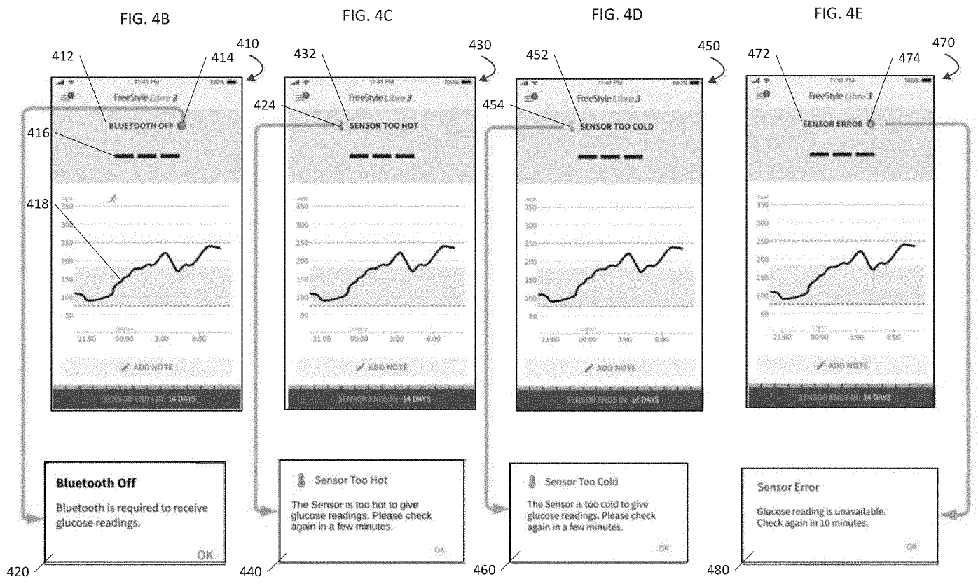

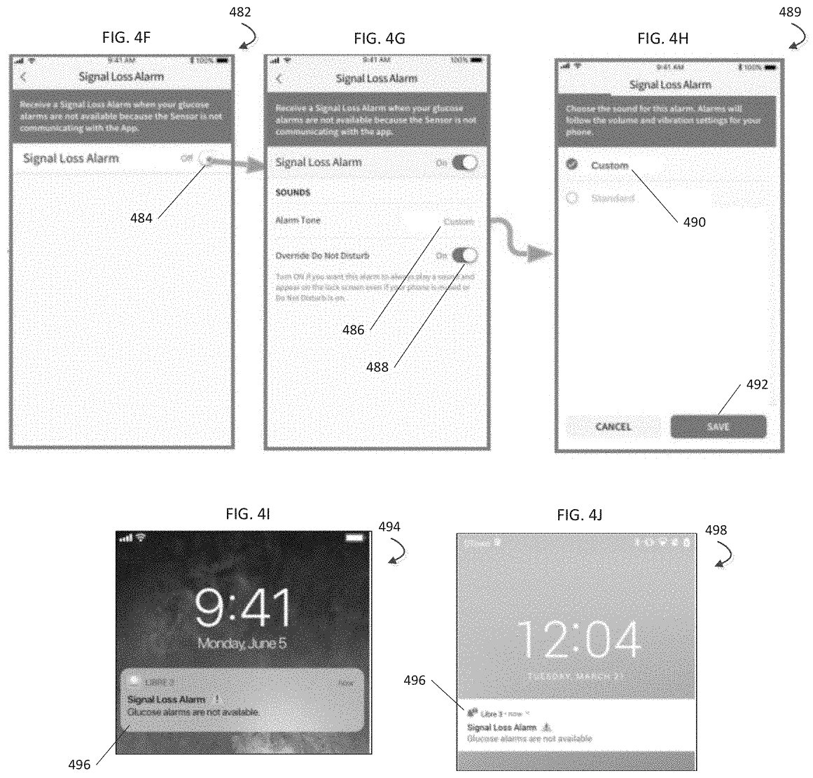

FIG. 3A is a flow diagram depicting an example embodiment of a method 300 for determining a signal loss condition. At Step 302, a current sensor reading is received. According to many of the embodiments, the current sensor reading can be received by a reader device such as, for example, a smart phone. In other embodiments, the current sensor reading can be received by a sensor control device, by a local computing system, or by any other device that is capable of communicating with an analyte sensor or sensor control device. At Step 304, a time elapsed since the last current sensor reading was received is determined. In some embodiments, this step can be performed by obtaining a current time from a clock of the device (or, alternatively, from a clock in communication with the device, e.g., a network time server) and comparing the current time with a timestamp associated with the last received current sensor reading. At Step 306, the time elapsed since the last received current sensor reading is compared to a predetermined signal loss indicator threshold. In some embodiments, for example, the signal loss indicator threshold can be a time period that is equal to or less than twenty minutes (e.g., 1, 2, 3, 4, 5, 10, or 15 minutes). At Step 308, if the time elapsed is not greater than the signal loss indicator threshold, then method 300 returns to Step 304 to determine the next time elapsed value. If the time elapsed is greater than the signal loss indicator threshold, then a signal loss indicator is generated. According to some embodiments, for example, the signal loss indicator can be a visual message or notification that is output to a display of the device. In some embodiments, the signal loss indicator message can comprise part of a sensor results screen (such as the embodiments shown in FIG. 4A). In other embodiments, the signal loss indicator can comprise a new screen, a pop-up window, or a banner notification that can either persist on the display until dismissed by the user or configured to disappear after a certain amount of time. In still other embodiments, the signal loss indicator can be an auditory or vibratory signal output to the device.

Although not shown, according to some embodiments, once a signal loss condition has been resolved (e.g., a new current sensor reading is received), the device can receive historical sensor readings to backfill any missing data on the device. Further details on data backfilling are described in the '672 Publication, which is hereby incorporated by reference in its entirety for all purposes.

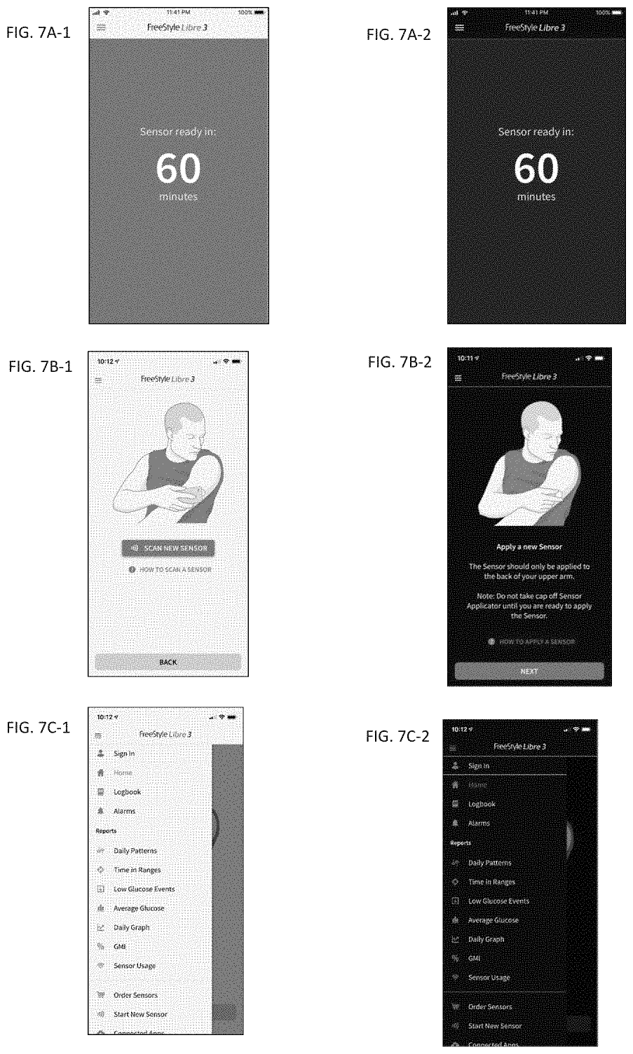

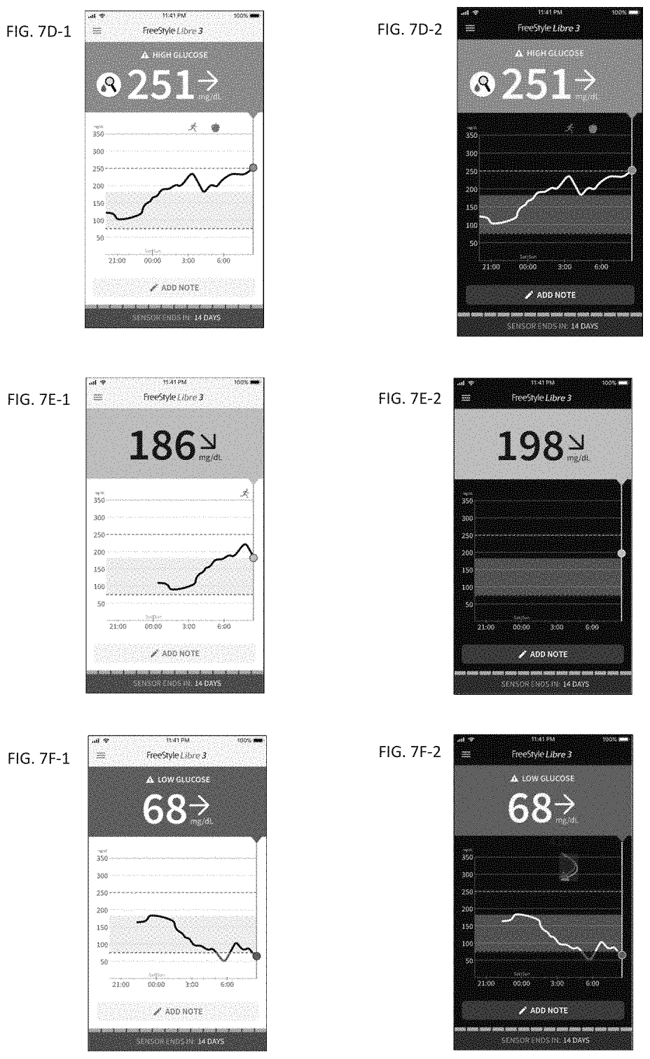

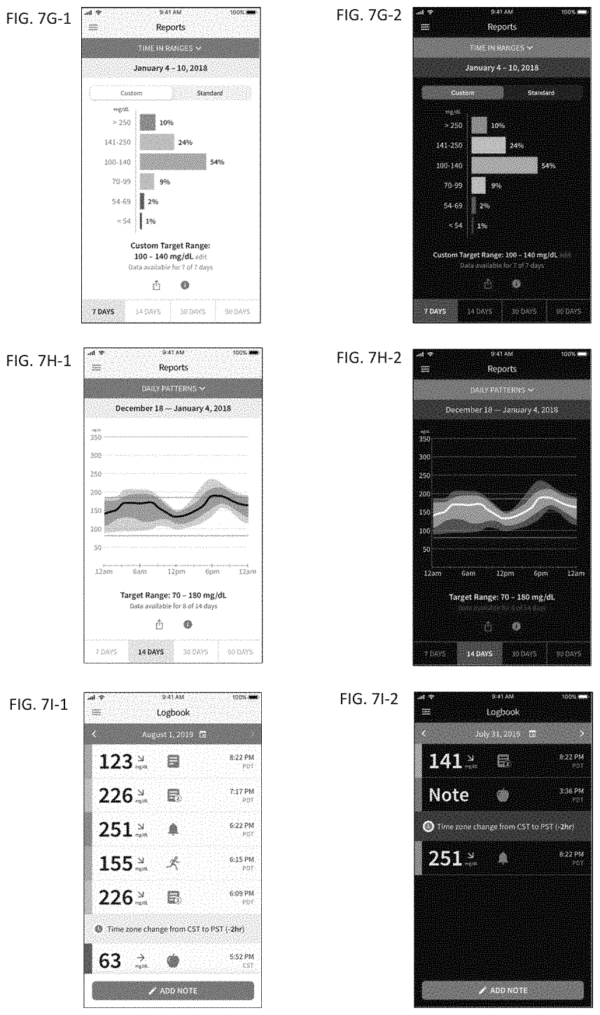

FIG. 3B is a flow diagram depicting another example embodiment of a method 310 for determining a signal loss condition. Like the example embodiment of method 300 (as described with respect to FIG. 3A), method 310 can also include the steps of receiving a current sensor reading at Step 312; determining a time elapsed since the last current sensor reading was received at Step 314; comparing the time elapsed since the last received current sensor reading to a predetermined signal loss indicator threshold at Step 316; and, in response to determining that the time elapsed is greater than a signal loss indicator threshold, generating a signal loss indicator at Step 318. An example of a signal loss indicator is described below with respect to FIG. 4A. According to an aspect of the embodiments, at Step 320, method 310 can further include a step of determining whether the current sensor reading is valid. If the current sensor reading is determined to be valid, then the valid current sensor reading is output to a display of the device at Step 324. (See, for example, FIG. 7E-1.) If, however, the current sensor reading is invalid, then an invalid sensor reading indicator is generated at Step 322. By way of example only, if the current sensor reading is invalid because the temperature of the sensor was too high, an invalid sensor reading indicator can output a message to the display of the device indicating the sensor was too hot to provide an accurate sensor reading. (See, for example, FIG. 4C.) Other exemplary invalid sensor reading indicators are described below with respect to FIGS. 4A to 4E below.

FIG. 3C is a flow diagram depicting an example embodiment of a method 330 for determining a no recent valid sensor reading condition. At Step 332, a current sensor reading is received by the device (e.g., reader device or smart phone). At Step 334, it is determined whether the current sensor reading is valid. If the current sensor reading is valid, then the valid current sensor reading is output to the display of the device at Step 336. (See, for example, FIG. 7E-1.) According to method 330, if the current sensor reading is invalid, then an invalid reading indicator is generated at Step 338. (See, for example, FIG. 4C.)

Referring still to FIG. 3C, at Step 340, a time elapsed since the last valid current sensor reading is determined. In some embodiments, this step can be performed by obtaining a current time from a clock of the device (or, alternatively, from a clock in communication with the device, e.g., a network time server) and comparing the current time with a timestamp associated with the last valid current sensor reading. At Step 342, the time elapsed since the last valid current sensor reading is compared to a predetermined no recent valid sensor reading alarm threshold. In some embodiments, for example, the no recent valid sensor reading alarm threshold can be a time period that is greater than five minutes (e.g., 10, 15, 20, 25, or 30 minutes). Furthermore, according to some embodiments, the no recent valid sensor reading alarm threshold can be user configurable. If it is determined that the time elapsed is not greater than the alarm threshold, then method 330 returns to Step 332 to receive the next current sensor reading. If, however, the time elapsed is greater than the alarm threshold then, at Step 344, a no recent valid sensor reading alarm is generated. According to some embodiments, the no recent valid sensor reading alarm can comprise a visual notification (e.g., pop-up, banner, new screen) that is output to a display of the device. (See, for example, FIGS. 4I and 4J.)

FIG. 3D is a flow diagram depicting an example embodiment of a method 350 for prioritizing and generating various error and analyte level condition indicators relating to current sensor readings. At Step 352, a current sensor reading is received by the device (e.g., reader device or smart phone). At Step 354, a time elapsed since the last current sensor reading was received is determined. In some embodiments, this step can be performed by obtaining a current time from a clock of the device (or, alternatively, from a clock in communication with the device, e.g., a network time server) and comparing the current time with a timestamp associated with the last received current sensor reading. At Step 356, the time elapsed since the last received current sensor reading is compared to a predetermined signal loss indicator threshold. In some embodiments, for example, the signal loss indicator threshold can be a time period that is equal to or less than twenty minutes (e.g., 1, 2, 3, 4, 5, 10, or 15 minutes). At Step 358, if the time elapsed is greater than the signal loss indicator threshold, then a signal loss indicator is generated. If, however, the time elapsed is not greater than the signal loss indicator threshold, then method 350 proceeds to check for various errors relating to current sensor readings in a predetermined order of priority. At Step 360, it is determined if an error is present relating to the sensor signal. For example, a software algorithm on the sensor control device can determine whether an early signal attenuation condition has been detected, and if so, can transmit this information to the device either as a separate packet or as part of the current sensor reading. If an error relating to the sensor signal is detected then, at Step 362, a sensor error indicator can be generated. (See, for example, FIG. 4E.)

If no error relating to the sensor signal is detected, then method 350 proceeds to Step 364 to determine if an error relating to the temperature of the sensor or the skin is present. According to one aspect of the embodiments, the sensor control device can include a temperature sensing element (e.g., a thermistor) capable of sensing the temperature of the sensor or the skin. In some embodiments, the sensor control device can transmit raw temperature readings to the device, wherein the device can determine if the received temperature readings exceed one or more predetermined temperature thresholds. In other embodiments, the sensor control device itself can determine if the temperature readings exceed the one or more predetermined temperature thresholds and, if so, transmit an indication to the device (either as part of the current sensor reading or as a separate packet) only if the thresholds are exceeded. If an error relating to the temperature of the sensor or the skin is detected then, at Step 366, a temperature error indicator can be generated. (See, for example, FIGS. 4C and 4D.)

If no error relating to the temperature of the sensor or the skin is detected, then method 350 proceeds to Step 368 to determine if an analyte level condition is present. In some embodiments, for example, the analyte level condition can comprise one or more of a glucose level that is outside of a non-configurable glucose level range, a glucose level above a high glucose level threshold, a glucose level below a low glucose level threshold, a projected glucose level above a high projected glucose level threshold, a projected glucose level below a low projected glucose level threshold, a glucose level within a user's configured target range, or a glucose level outside a user's configured target range. If it is determined that an analyte level condition is present then, at Step 370, an analyte condition indicator is generated. (See, for example, FIGS. 7D-1 and 7F-1.) If it is determined that no analyte level condition is present then, at Step 372, the valid current sensor reading is output to the display of the device. (See, for example, FIG. 7E-1.)

Although the aforementioned conditions refer to glucose level conditions, those of skill in the art will appreciate that conditions relating to other analyte levels and thresholds (e.g., ketone levels, lactate levels, etc.) can also be implemented with any of the example embodiment methods described herein, and are fully within the scope of the present disclosure.

It will also be understood by those of skill in the art that the order of priority depicted in FIG. 3D, and as described with respect to the aforementioned errors and analyte level conditions, are merely illustrative. In other embodiments not shown, for example, temperature error determination can occur before the determination of sensor signal errors. Other combinations and orders of priority are possible, and are fully within the scope of the present disclosure.

FIGS. 4A to 4J are example embodiments of GUIs for use with the methods described with respect to FIGS. 3A to 3D.

FIG. 4A is a GUI 400 depicting a signal loss indicator 402 as part of a sensor results screen. According to one aspect of the embodiments, signal loss indicator 402 can indicate that a current sensor reading has not been received within the time period comprising a signal loss indicator threshold. According to another aspect of the embodiments, signal loss indicator 402 can be accompanied by placeholder characters (e.g., three dashes 404) in place of a numeric analyte level value and trend arrow indicator, as shown in FIG. 7E-1. In addition, according to some embodiments, the signal loss indicator 402 can include an informational icon that, when pressed by the user, can cause an informational window (408A or 408B) to appear with additional information and user guidance relating to the error condition. According to another aspect of the embodiments, GUI 400 can further include information relating to historical sensor readings, such as a glucose trend line 406. In this manner, the user is notified of a current signal loss condition but can also review previous historical sensor readings.

FIGS. 4B to 4E are example embodiments of GUIs including various error indicators as part of sensor results screens. FIG. 4B is a GUI 410 depicting a Bluetooth Off indicator 412, to indicate that the Bluetooth transmitter of the device has been disabled or switched off. The Bluetooth Off indicator 412 can also be accompanied by placeholder characters 416 (in place of a numeric analyte level and trend arrow indicator), as well as an information icon 414 that, when pressed by the user, causes an informational window 420 to appear with additional information and user guidance relating to the error condition. GUI 410 further includes information relating to historical sensor readings, such as glucose trend line 418.

FIGS. 4C and 4D are GUIs 430 and 450, respectively, depicting a Sensor Too Hot indicator 432 and a Sensor Too Cold indicator 452, each indicating an error relating to the temperature of the sensor or the temperature of the skin. Indicators 432 and 452 are further accompanied by placeholder characters, as well as informational icons 424, 454 (shown as a thermometer icon) that, when pressed by the user, causes informational windows 440, 460 to appear with additional information and user guidance relating to the error condition. GUIs 430 and 450 can also include information relating to historical sensor readings such as, for example, glucose trend lines.

FIG. 4E is a GUI 470 depicting a sensor error indicator 472 to indicate an error relating to the sensor signal. In some embodiments, the sensor error can be an error detected on the sensor control device relating to the sensor signal. For example, the error can be associated with the detection of early signal attenuation. As another example, the error can be associated with a rate of change metric beyond a predetermined threshold, as measured by the sensor control device. Those of skill in the art will appreciate that the sensor error indicator 472 can indicate other signal-related errors diagnosed by the sensor control device and are fully within the scope of the present disclosure. According to another aspect of the embodiments, indicator 472 is accompanied by informational icon 474 that, when pressed by the user, causes informational window 480 to appear with additional information and user guidance relating to the error condition. For example, information window 480 can include an instruction to the user to check for an analyte reading after a predetermined period of time (e.g., ten minutes). According to some embodiments, the predetermined period of time presented in information window 480 can change depending on a time remaining until the error condition is expected to resolve. In other embodiments, the predetermined period of time can be a static value.

FIGS. 4F to 4H are GUIs 482 and 489 depicting interfaces for allowing a user to configure the no recent valid sensor reading alarm (sometimes also referred to as a “Signal Loss Alarm”). Referring first to FIG. 4F, GUI 482 includes a switch 484 that can be toggled between an “On” position and an “Off” position in order to activate or de-activate, respectively, the Signal Loss Alarm. If the switch is toggled to the “On” position, additional configuration settings are presented, as shown in FIG. 4G. According to another aspect of the embodiments, these additional settings can include an alarm tone setting 486 and an override do not disturb switch 488. In some embodiments, if the override do not disturb switch 488 is enabled, then a signal loss alarm will be presented even if the device operating system's do not disturb mode is enabled. In addition, according to other embodiments, the alarm tone setting 486, when pressed, can cause GUI 489 to be displayed, wherein GUI 489 includes a plurality of radio buttons 490 to allow the user to select a specific alarm tone (e.g., custom, standard, etc.).

FIGS. 4I and 4J are GUIs 494 and 498 depicting alarm interfaces for the no recent valid sensor reading alarm, or “Signal Loss Alarm.” As shown in the figures, the alarm interface 496 can comprise a window or banner notification that includes the text, “Signal Loss Alarm, Glucose alarms are not available.” According to some embodiments, alarm interface 496 can be configured to appear whether the device is currently in a locked state, a state in which a third-party application (e.g., YouTube) is in the foreground, or a state in which the analyte monitoring software is in the foreground. In some embodiments, alarm interface 496 can be configured as a temporary notification which can disappear after a predetermined amount of time without user interaction. In other embodiments, alarm interface 496 can persist on the display until a user has acknowledged or dismissed the alarm.

### Example Embodiments of Methods for Generating Time-in-Ranges Interfaces

Example embodiments of methods for generating Time-in-Ranges interfaces will now be described. According to one aspect of the embodiments, a Time-in-Ranges interface can provide useful information to a patient trying to monitor and manage his or her analyte levels by presenting information in a histogram format, which can provide a unique perspective of a patient's glycemic control. In many of the embodiments, a Time-in-Range interface provides a percentage of time within a predetermined reporting period that the patient's analyte levels were within a specific analyte level range. In addition, according to some embodiments, some of the analyte level ranges can be based on consensus standards, while others can be customized to fit the patient's personal goals. Further details regarding Time-in-Range interfaces are described in the '672 Publication, which is hereby incorporated by reference in its entirety for all purposes.

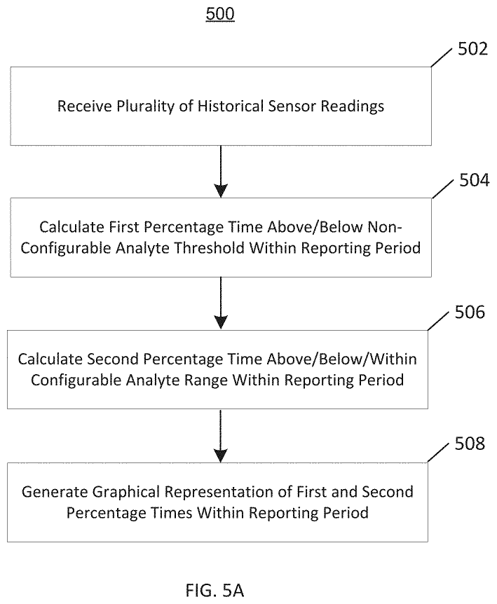

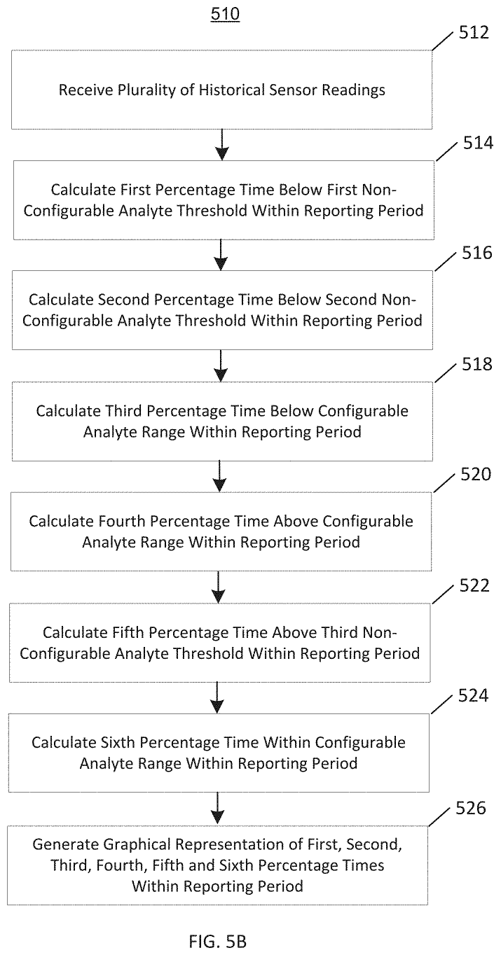

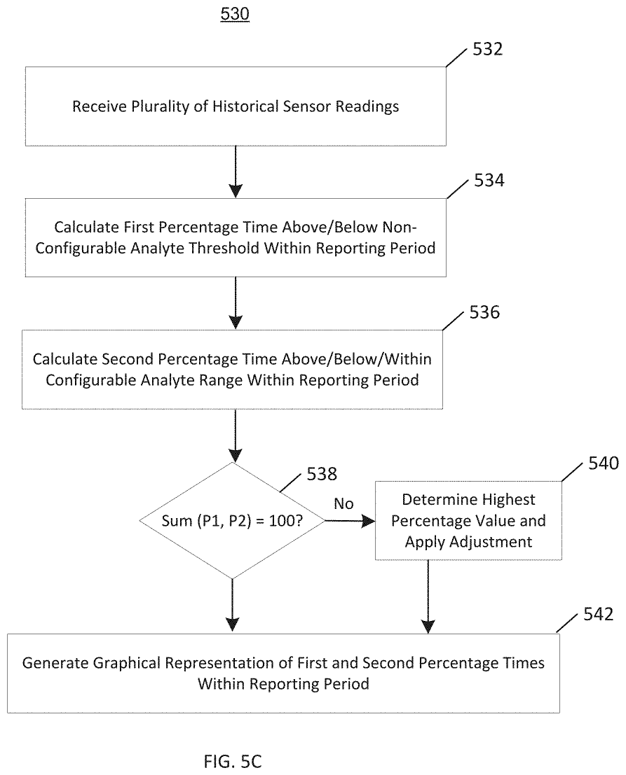

FIG. 5A is a flow diagram depicting an example embodiment of a method 500 for generating a Time-in-Ranges interface. At Step 502, a plurality of historical sensor readings is received. According to many of the embodiments, the plurality of historical sensor readings can be received by a reader device such as, for example, a smart phone. In other embodiments, the plurality of historical sensor readings can be received by a sensor control device, by a local computing system, or by any other device that is capable of communicating with an analyte sensor or sensor control device. At Step 504, a first percentage of time above (or below) a non-configurable analyte threshold can be calculated using the received plurality of historical sensor readings for a predetermined reporting period. In some embodiments, for example, the predetermined reporting period can be a date range that is configurable by the user. At Step 506, a second percentage of time either above, below, or within a user-configurable analyte range can be calculated using the same received plurality of historical sensor readings for the predetermined reporting period. At Step 508, a graphical representation of the first and second percentage times within the predetermined reporting period is generated and outputted to a display of the device. In this manner, method 500 provides for a Time-in-Range interface that can comprise information about a patient's glycemic control according to both a non-configurable threshold (e.g., according to a consensus standard) and a user-configurable threshold or range.

FIG. 5B is a flow diagram depicting an example embodiment of a method 510 for generating another Time-in-Ranges interface. At Step 512, a plurality of historical sensor readings is received by a device, such as, for example, a reader device or smart phone. At Step 514, a first percentage of time below a first non-configurable analyte threshold can be calculated using the received plurality of historical sensor readings for a predetermined reporting period. By way of example only, in some embodiments, the first non-configurable analyte threshold can comprise a threshold of 54 mg/dL (3 mmol/L) that cannot be changed by the user. At Step 516, a second percentage of time below a second non-configurable analyte threshold can be calculated using the received plurality of historical sensor readings for the predetermined reporting period. According to some embodiments, the second non-configurable analyte threshold can comprise a threshold of 70 mg/dL (3.9 mmol/L) that cannot be changed by the user.

Referring still to FIG. 5B, at Step 518, a third percentage of time below a configurable analyte range can be calculated using the received plurality of historical sensor readings for the predetermined reporting period. At Step 520, a fourth percentage of time above the configurable analyte range can be calculated using the received plurality of historical sensor readings for the predetermined reporting period. According to one aspect of the embodiments, the configurable analyte range can comprise a target glucose range (e.g., an upper target analyte level and a lower target analyte level) that can be changed by the user.

At Step 522, a fifth percentage of time above a third non-configurable analyte threshold can be calculated using the received plurality of historical sensor readings for the predetermined reporting period. By way of example only, in some embodiments, the third non-configurable analyte threshold can comprise a threshold of 250 mg/dL (13.9 mmol/L) that cannot be changed by the user. At Step 524, a sixth percentage of time within the configurable analyte range can be calculated using the received plurality of historical sensor readings for the predetermined reporting period. As described above, the configurable analyte range can comprise a target glucose range (e.g., an upper target analyte level and a lower target analyte level) that can be changed by the user.

At Step 526, a graphical representation of the first, second, third, fourth, fifth, and sixth percentages of times based on the received plurality of historical sensor readings can be generated for the predetermined reporting period. According to some embodiments, for example, each of the percentages of times can be graphically represented as an individual bar, with the length of each bar proportional to the percentage value. In other embodiments, the percentages of times can be graphically represented as bar portions of a single bar, with the length of each bar portion proportional to the percentage value. In still other embodiments, the percentages of time can be graphically represented as portions or segments of a circle (e.g., a pie chart), with the area of each portion or segment proportional the percentage value.

Although the example embodiment of FIG. 5B describes calculating and generating graphical representations of six percentages of time, wherein three of the percentages of time associated with non-configurable thresholds/ranges and three of the percentages of time are associated with configurable thresholds/ranges, those of skill in the art will recognize that method 510 can be used to calculate and generate graphical representations of any number of percentages of time, having any combination of non-configurable and configurable ranges and/or thresholds. Further details of graphical representations of Time-in-Ranges interfaces are described in '672 Publication, which is hereby incorporated by reference in its entirety for all purposes.

FIG. 5C is a flow diagram depicting an example embodiment of a method 530 for generating a Time-in-Ranges interface to account for historical sensor readings that do not fall into one of the configurable or non-configurable threshold/ranges. In certain circumstances, one or more historical sensor readings may not fall into one of the configurable or non-configurable thresholds/ranges such that the sum of the percentages of time do not equal a hundred percent. For example, one or more historical sensor readings may comprise a non-numeric value (e.g., an error condition) or a numeric value that does not fall into any of the configurable or non-configurable thresholds/ranges. In one aspect, method 530 can apply an adjustment to account for these situations.

At Step 532, a plurality of historical sensor readings is received by a device, such as, for example, a reader device or a smart phone. At Step 534, a first percentage of time above (or below) a non-configurable analyte threshold can be calculated using the received plurality of historical sensor readings for a predetermined reporting period. At Step 536, a second percentage of time above, below, or within a user-configurable analyte range can be calculated using the same received plurality of historical sensor readings for the predetermined reporting period. At Step 538, it is determined if the sum of the first and the second percentages of time equal a hundred. If so, then method 530 proceeds to Step 542, where a graphical representation of the first and the second percentage times are generated for the reporting period. If the sum of the first and the second percentages of time do not equal a hundred, then method 530 proceeds to Step 540. At Step 540, it is determined which of the percentages of time has the highest value. Subsequently, an adjustment to the percentage of time with the highest value is made. In some embodiments, for example, the percentage of time with the highest value is adjusted such that the sum of the first and the second percentages of time equal a hundred. In other embodiments, also by way of example, the percentage of time with the highest value is adjusted by multiplying it by a factor. In still other embodiments, the lowest value of percentages of time is adjusted such that the sum of the percentages of time equal a hundred. After the adjustment is applied at Step 540, method 530 proceeds to Step 542, where a graphical representation of the first and the second percentage times are generated for the reporting period.

Although the example embodiment of FIG. 5C is described with a first and a second percentage of time, those of skill in the art will appreciate that method 530 can be applied to any number of percentages of time. By way of example, method 530 can be used to adjust percentages where there are six percentages of time, as described with respect to FIG. 5B.

According to another aspect of the embodiments, in circumstances where multiple percentages of time share the highest value, the adjustment can be made to a single percentage based on a predetermined order of priority. For example, in some embodiments, an order of priority can be (from highest priority to lowest priority): percentage of time above 250 mg/dL, percentage of time above a target glucose range, percentage of time within a target glucose range, percentage of time below a target glucose range, percentage of time below 70 mg/dL, and percentage of time below 54 mg/dL.

### Example Embodiments Relating to Enhanced Visibility Mode

Example embodiments of methods and GUIs relating to an enhanced visibility mode will now be described. In certain environments, it may be desirable to enhance the display of a computing device for better visibility with respect to many of the digital and graphical interfaces described herein. For example, certain color shadings in a chart or graph may be difficult to see in a low light setting due to a lack of contrast between the colors. By way of another example, interfaces can be modified to utilize certain background colors which may provide less strain on the eyes in a low light setting.

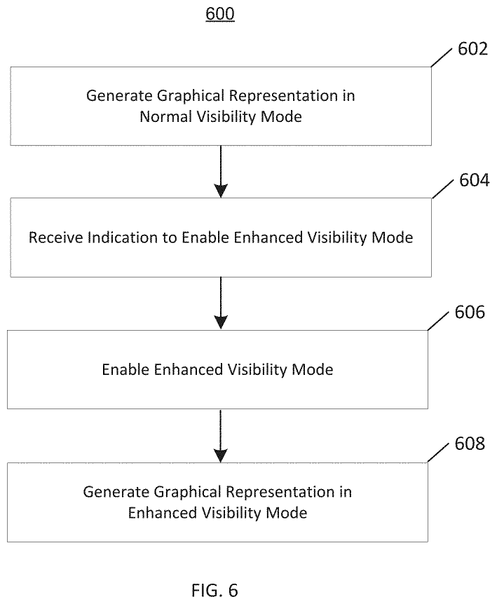

FIG. 6 is a flow diagram depicting an example embodiment of a method 600 for enabling an enhanced visibility mode. At Step 602, a graphical representation of a digital or graphical user interface, including any of the interfaces described herein, is generated according to a normal visibility mode and outputted to a display of a device. According to many of the embodiments, the device can be a reader device such as, for example, a smart phone. In other embodiments, device can be a local computing system or any other device that is capable of communicating with an analyte sensor or a sensor control device. At Step 604, an indication to enable an enhanced visibility mode is received by the device. According to one aspect of the embodiments, the indication to enable the enhanced visibility mode can be a user-configurable setting of the operating system of the device (e.g., iOS, Android). In other embodiments, the indication to enable the enhanced visibility mode can be a user-configurable setting within an analyte monitoring software application. In some embodiments, the indication to enable the enhanced visibility mode can be generated automatically in response to the detection of light above or below a certain predetermined activation threshold by a light sensor (e.g., photoelectric devices, photo sensors, phototransistors, photoresistors, and/or photodiodes). In still other embodiments, the indication to enable the enhanced visibility mode can be generated according to a predetermined time schedule (e.g., 6:00 PM to 6:00 AM) or according to a seasonal time schedule (e.g., sunset to sunrise). Those of skill in the art will understand that other activation mechanisms can be utilized and fully within the scope of the present disclosure.

Referring still to FIG. 6, at Step 606, the enhanced visibility mode is enabled on the device. According to some embodiments, the enhanced visibility mode can remain enabled until it is manually disabled by the user. In other embodiments, the enhanced visibility mode can be disabled after the expiration of a timer or, in the alternative, according to a predetermined time schedule or a seasonal time schedule. In still other embodiments, the enhanced visibility mode can be disabled in response to the detection of light above or above a certain predetermined deactivation threshold by a light sensor. Subsequently, at Step 608, a graphical representation of a digital or graphical user interface, including any of the interfaces described herein, is generated according to the enhanced visibility mode and outputted to the display of the device.

FIGS. 7A-1 to 7I-2 are GUIs depicting interfaces for an analyte monitoring software application. In particular, each GUI is shown in normal visibility mode and enhanced visibility mode. FIGS. 7A-1 and 7A-2 are GUIs depicting a sensor warm-up interface for an analyte monitoring software application, displayed according to a normal visibility mode and an enhanced visibility mode, respectively. FIGS. 7B-1 and 7B-2 are GUIs depicting a home screen for an analyte monitoring software application, displayed according to a normal visibility mode and an enhanced visibility mode, respectively. FIGS. 7C-1 and 7C-2 are GUIs depicting a menu interface for an analyte monitoring software application, displayed according to a normal visibility mode and an enhanced visibility mode, respectively. FIGS. 7D-1, 7D-2, 7E-1, 7E-2, 7F-1, and 7F-2 are GUIs depicting sensor results interfaces for an analyte monitoring software application, displayed according to a normal visibility mode and an enhanced visibility mode. FIGS. 7G-1, 7G-2, 7H-1, 7H-2, 7I-1, and 7I-2 are GUIs depicting report interfaces for an analyte monitoring software application, displayed according to a normal visibility mode and an enhanced visibility mode. These depicted embodiments are intended to illustrate examples of GUIs and interfaces in normal visibility mode and enhanced visibility mode, and those of skill in the art will readily understand that the disclosure of the enhanced visibility mode is not limited to the particular embodiments shown and/or described.

### Example Embodiments Relating to Voice Accessibility Mode

Example embodiments of methods and GUIs relating to an enhanced accessibility mode will now be described. According to one aspect of the embodiments, a voice accessibility mode can be enabled to provide for audible descriptions of interfaces on a display of a device. In some embodiments, the voice accessibility mode can further include gesture recognition such that when a user touches the display (e.g., drags a finger over a portion of the display), the voice accessibility mode converts the touched portion of a display to an audible output. In this manner, voice accessibility mode can be configured to increase accessibility for blind and low-vision users, as well as for users with dyslexia.

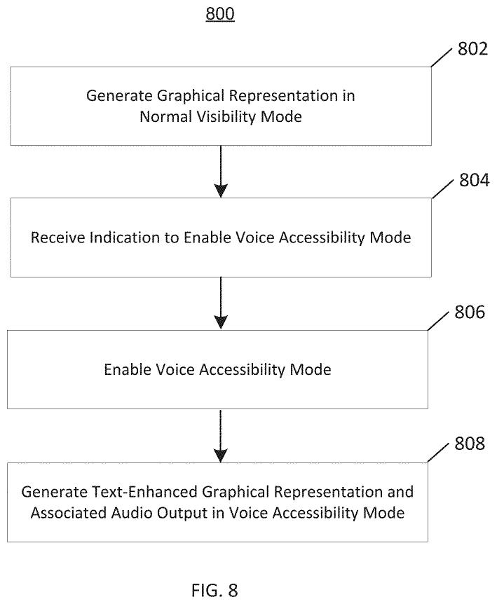

FIG. 8 is a flow diagram depicting an example embodiment of a method 800 for enabling a voice accessibility mode. At Step 802, a graphical representation of a digital or graphical user interface, including any of the interfaces described herein, is generated according to a normal visibility mode and outputted to a display of a device. According to many of the embodiments, the device can be a reader device such as, for example, a smart phone. In other embodiments, device can be a local computing system or any other device that is capable of communicating with an analyte sensor or a sensor control device. At Step 804, an indication to enable a voice accessibility mode is received by the device. According to one aspect of the embodiments, the indication to enable the voice accessibility mode can be a user-configurable setting of the operating system of the device (e.g., iOS, Android). In other embodiments, the indication to enable the voice accessibility mode can be a user-configurable setting within an analyte monitoring software application.

Referring still to FIG. 8, at Step 806, the voice accessibility mode is enabled on the device. According to some embodiments, the voice accessibility mode can remain enabled until it is manually disabled by the user. Subsequently, at Step 808, a text-enhanced graphical representation of a digital or graphical user interface and associated audible output is generated according to the voice accessibility mode. For example, in some embodiments, certain graphics, non-textual icons or indicators (e.g., trend arrow) on an interface can be replaced with descriptive text or a textual element, which can then be converted to an audible output by the voice accessibility mode. In other embodiments, certain gesture responsive portions of an interface can be configured to cause the device to convert the text to audible speech. In still other embodiments, certain touch responsive portions of the interface can be configured in groupings such that the device will convert the text in an entire grouping to audible speech in response to the user touching any portion associated with the grouping. In still other embodiments, the voice accessibility mode can be integrated with a virtual assistant (e.g., Siri, Alexa) such that the user need not provide any touch-based input.

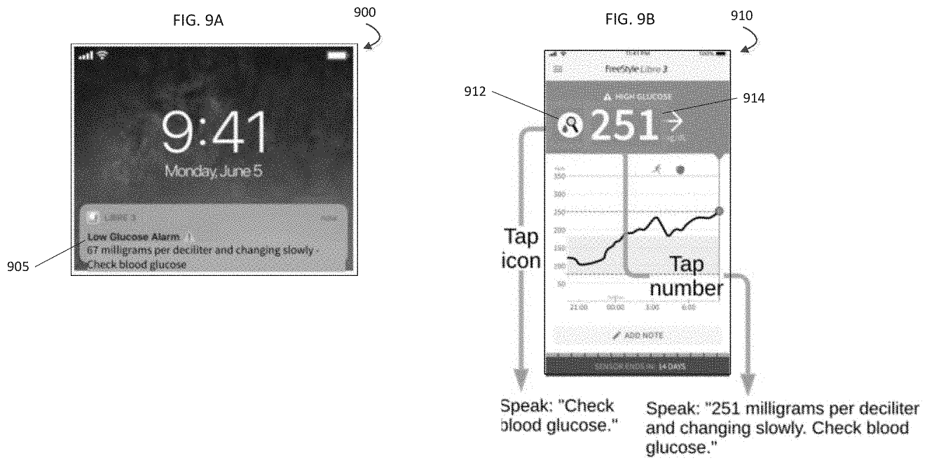

FIG. 9A is a GUI 900 depicting a low glucose alarm interface 905 on a display of a smart phone where the voice accessibility mode has been enabled. According to one aspect of the embodiments, alarm interface 905 includes text modified by the voice accessibility mode. In particular, standard abbreviated terms, such as mg/dL, have been replaced with the unabbreviated terms. In addition, a non-textual trend arrow has been replaced with a descriptive text (e.g., “changing slowly”). According to another aspect of the embodiments, the voice accessibility mode can convert the modified textual portions into audible output.

FIG. 9B is another GUI 910 depicting a sensor results interface 910 of an analyte monitoring software application where the voice accessibility mode has been enabled. As indicated by the highlighted upper-left portion, if graphical icon 912 is touched by the user, an audible output is generated (e.g., speak: “Check blood glucose.”). According to another aspect of the embodiments, GUI 910 includes a grouping of different graphical elements of the interface such that if the user touches any portion of interface 914, an audible output is generated (e.g., speak “251 milligrams per deciliter and changing slowly. Check blood glucose.”)

### Additional Example Embodiments of Digital and User Interfaces for Analyte Monitoring

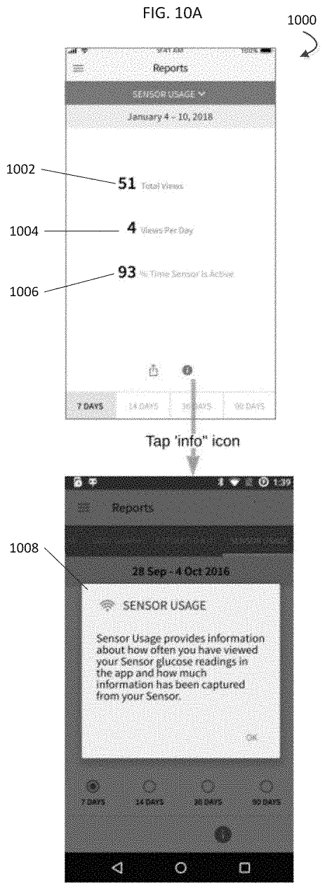

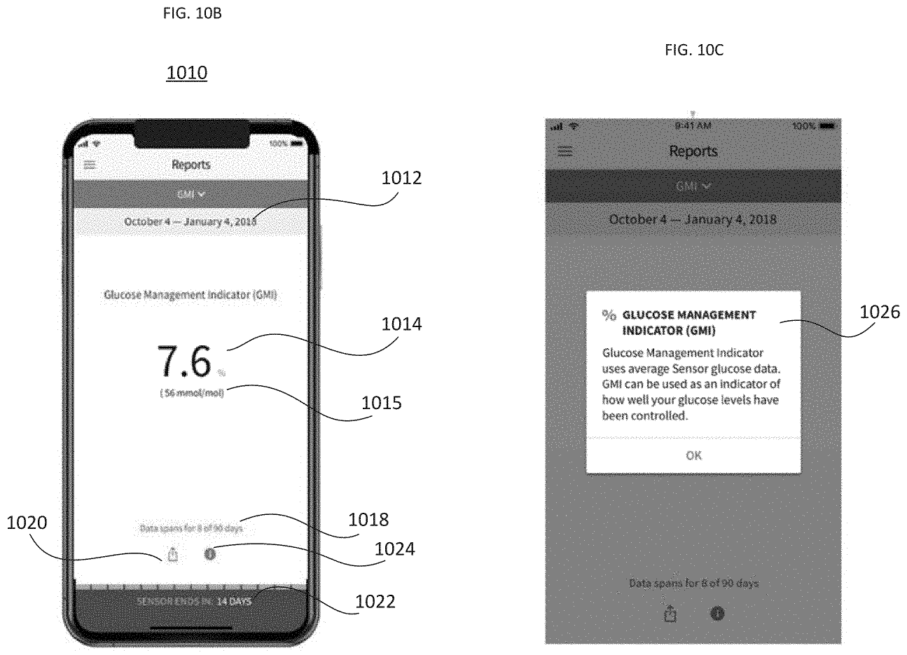

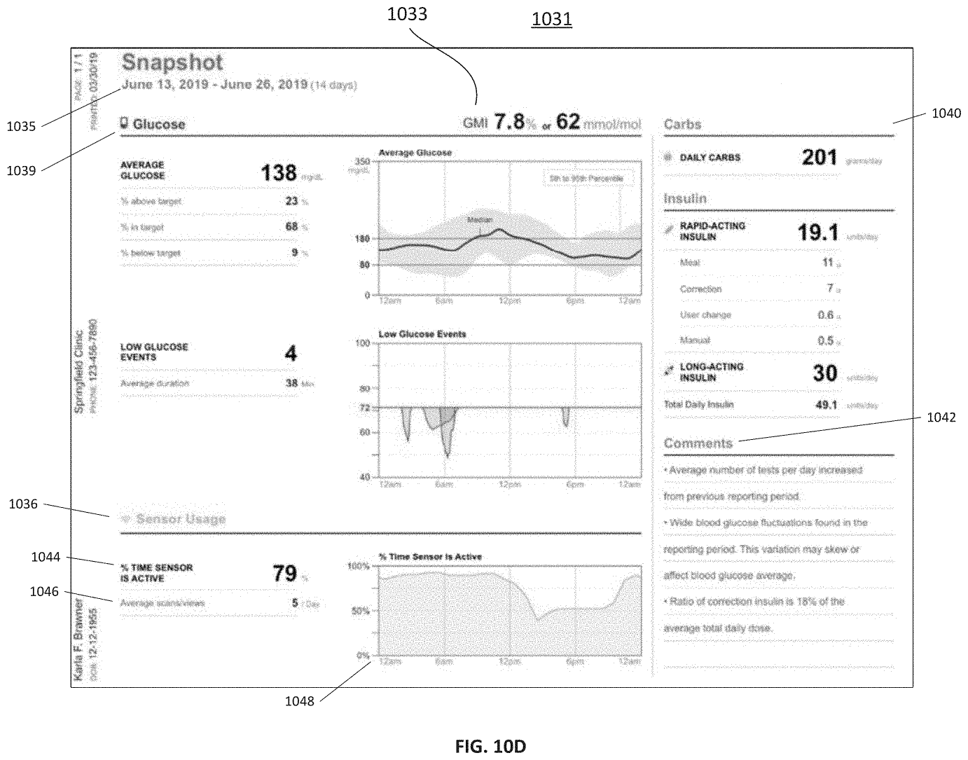

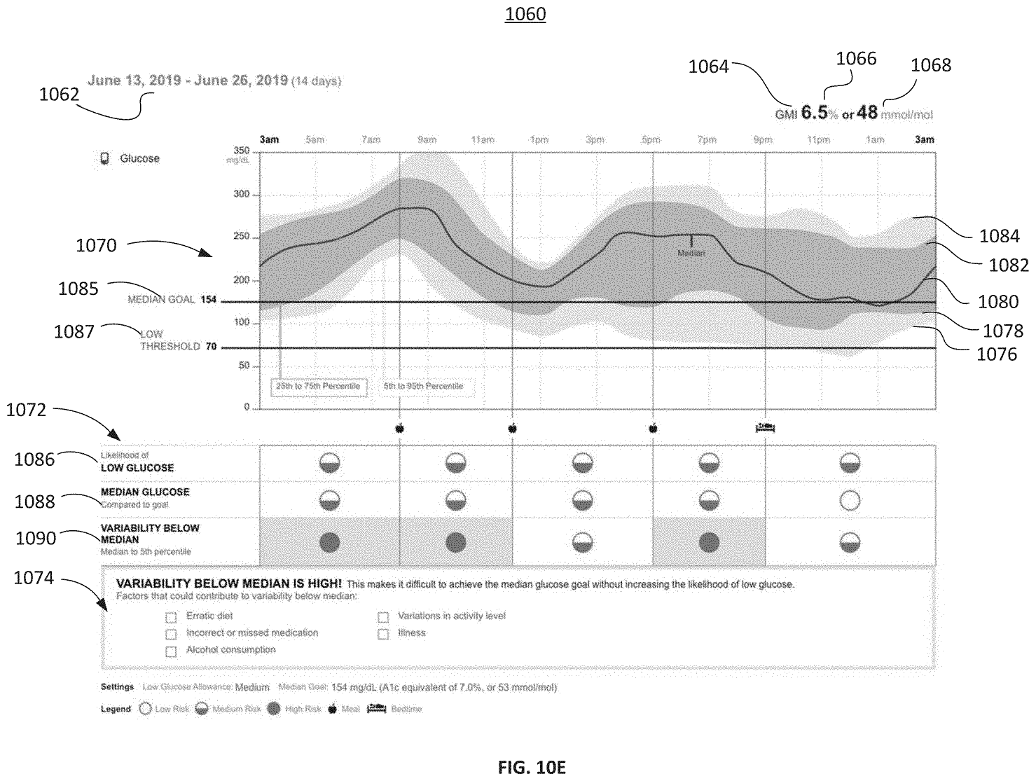

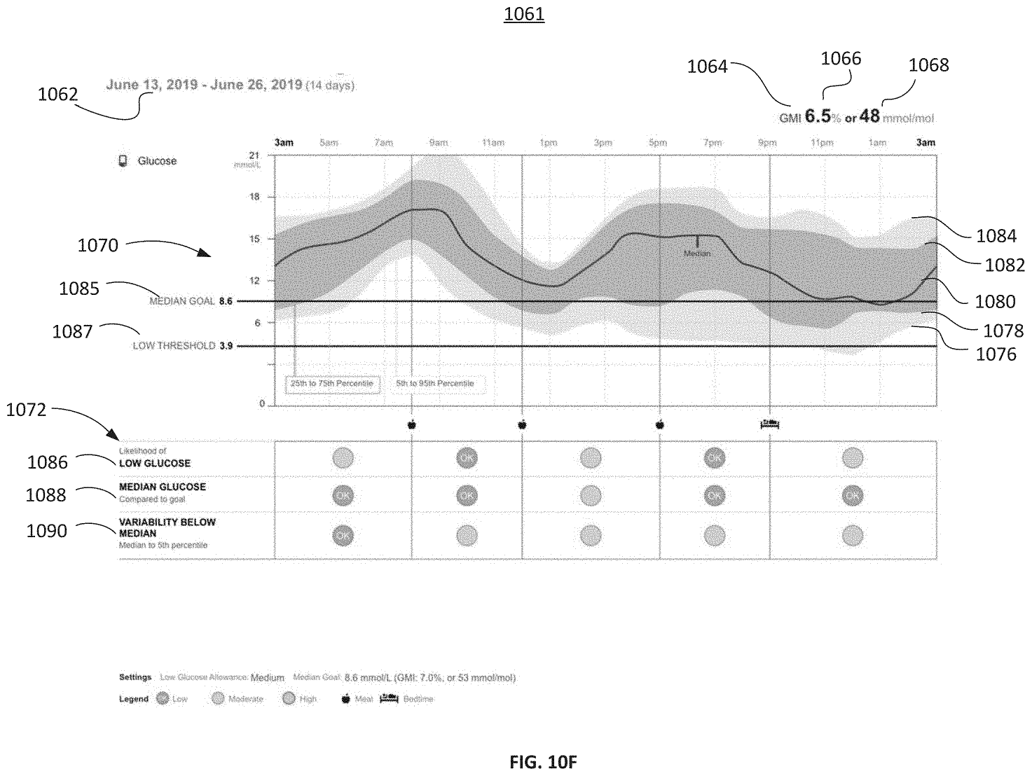

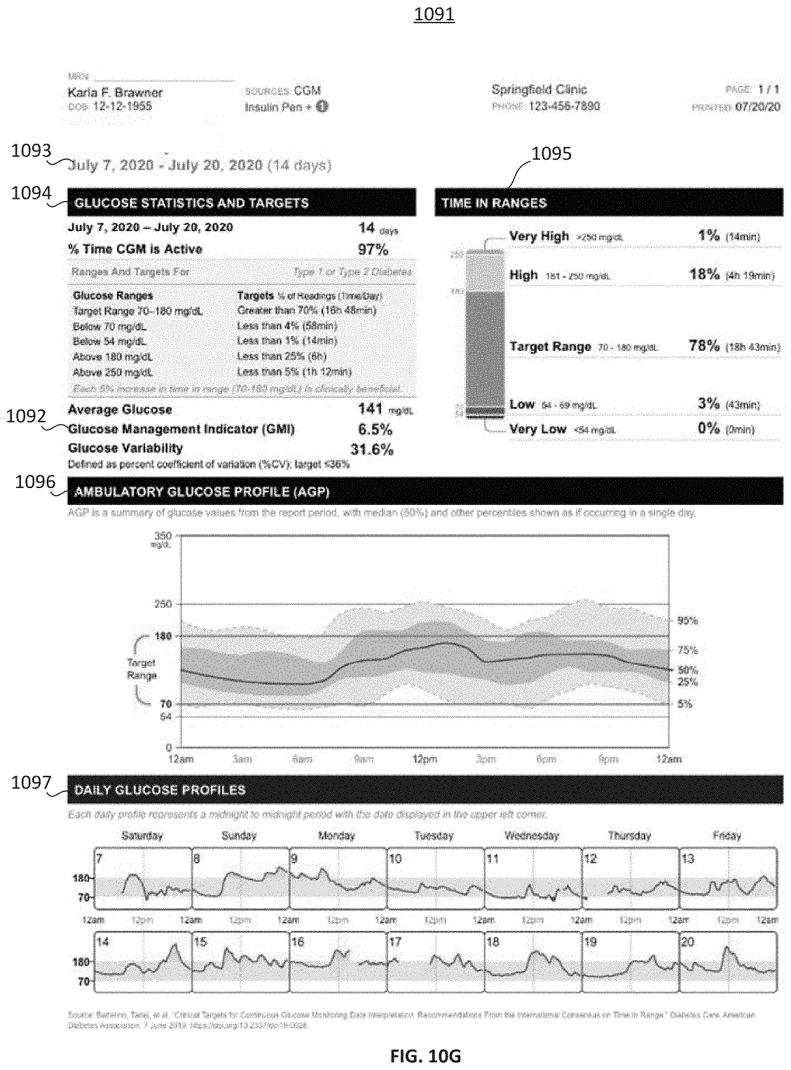

Additional example embodiments of digital and user interfaces for analyte monitoring will now be described. Referring first to FIG. 10A, GUI 1000 depicts a sensor usage report interface. According to one aspect of the embodiments, sensor usage report interface can comprise a Total Views metric 1002, which is indicative of a total number of views over a predetermined time period; a Views Per Day metric 1004, which is indicative of an average number of views per day over the predetermined time period; and a Percentage Time Sensor Active metric 1006, which is indicative of the percentage of predetermined time period that a device is in communication with sensor control device. In addition, GUI 1000 can include an information icon that, when pressed, causes an information window 1008 to appear with additional information relating to sensor usage information. According to one aspect of the embodiments, a “view” can be defined as an instance in which a sensor results interface is rendered or brought into the foreground. According to other embodiments, a “view” can be defined as an instance when a user views a sensor results interface with a valid sensor reading for the first time in a sensor lifecount. Further details regarding view metrics are described in the '672 Publication, which is hereby incorporated by reference in its entirety for all purposes.

FIGS. 10B to 10G depict various interfaces relating to Glucose Management Indicator, or GMI. Hemoglobin A1C has been used as a measure of a patient's average blood glucose levels over the past three months. It is often used as an indicator as to how well a patient is managing their diabetes, or is used when testing for diabetes or pre-diabetes. It is also a risk marker for diabetes complications. In light of the increasing use of continuous glucose monitoring and flash monitoring systems, a new marker, “Glucose Management Indicator” or “GMI” was established based on converting a mean glucose from continuous or flash monitoring systems based on population data used from clinical trials using CGM systems. See Bergenstal, R. M et al., “Glucose Management Indicator (GMI): A New Term for Estimating AIC From Continuous Glucose Monitoring.” Diabetes Care 41(11): 2275-80 (November 2018), which is hereby expressly incorporated by reference in its entirety for all purposes.

The GMI may be reported as a percentage for a reporting period or as a value in mmol/mol for a reporting period. The GMI percentage for a reporting period can be calculated according to formula (1). The GMI value in mmol/mol can be calculated according to formula (2).

GMI(%)=round_to_0.1{3.31+0.02392*(G_{avg})} (1)

GMI(mmol/mol)=round{12.71+4.70587*(G_{avg})} (2)

FIG. 10B depicts an example embodiment of a user interface relating to GUIs for analyte monitoring systems. According to one aspect of the embodiments, user interface 1010 can be rendered and displayed, for example, by a mobile app or software residing in non-transitory memory of reader device 120, such as those described with respect to FIGS. 1 and 2A. Referring to FIG. 10B, user interface 1010 can comprise: a time period interval 1012 indicative of a time period (e.g., a date range) during which a GMI metric is determined. The GMI metric 1014, 1015 may be displayed as a GMI percentage, a GMI value in mmol/mol, or both. An additional indication as to the amount of days for which data is being considered 1018 in the time period interval 1012 may also be displayed. In one embodiment, user interface 1010 can include one or more GMI metrics 1014, 1015. In another embodiment, user interface 1010 can include a GMI percentage 1014 and a GMI value in mmol/mol 1015 for the time period 1012. Where more than one GMI metric is displayed, one of the GMI metrics may be more prominently displayed than the other GMI metric. For example, the GMI percentage 1014 may be displayed in a larger font or a different color than the GMI value in mmol/mol 1015. The user interface 1010 may also include an indication as to the life of the current sensor remaining. For example, a statement 1022 that the sensor ends in a certain number of days may be displayed at the bottom. The user interface 1010 may also include an option 1020 to share or otherwise save the one or more GMI metrics. The user interface 1010 may also include a link (e.g., “i”) 1024 to click on for more information on GMI. After a user clicks on link 1024, a GUI 1026 may be displayed with a description of GMI. For example, as seen in FIG. 10C, the description may explain that GMI uses average sensor glucose data and can be used as an indicator of how well a patient's glucose levels have been controlled.

FIG. 10D depicts an example embodiment of a GMI metric 1033, as part of analyte monitoring system report GUI 1031. According to one aspect of the embodiments, GUI 1031 can be displayed on a computing device (e.g., personal computer, desktop computer, laptop computer, reader device, tablet computer, etc.) through a web browser, a web-enabled client, or software residing in non-transitory memory of the computing device. In some embodiments, where the user interface is displayed through a web browser or a web-enabled client, software instructions can be executed on a remote server (or a group of remote servers) which, in turn, can cause the web browser or web-enabled client residing on the computing device to display the user interface. According to one aspect of the embodiments, GUI 1031 is a snapshot report covering a predetermined time period 1035 (e.g., 14 days), and including a plurality of report portions on a single report GUI, including: a GMI metric 1033, a sensor usage interface portion 1036, a glucose trend interface 1039, which can include an glucose trend graph, a low glucose events graph, a health information interface 1040, which can include information logged by the user about the user's average daily carbohydrate intake and medication dosages (e.g., insulin dosages); and a comments interface 1042, which can include additional information about the user's analyte and medication patterns presented in a narrative format. GMI metric 1033 can be reported as either a GMI percentage, a GMI value in mmol/mol, or both. According to another aspect of the embodiments, sensor usage interface 1036 can comprise a Percentage Time Sensor Active metric 1044, an Average Scans/Views metric 1046 (e.g., indicative of an average sum of a number of scans and a number of views), and a Percentage Time Sensor Active graph 1048. As can be seen in FIG. 10D, an axis of the Percentage Time Sensor Active graph can be aligned with a corresponding axis of one or more other graphs (e.g., average glucose trend graph, low glucose events graph), such that the user can visually correlate data between multiple graphs from two or more portions of the report GUI by the common units (e.g., time of day) from the aligned axes.

FIGS. 10E and 10F depict example embodiments of GMI metric 1064, as part of analyte monitoring system report GUI 1060, 1061, which provides insights regarding the patient's diabetes management. According to one aspect of the embodiments, GUI 1060, 1061 can be displayed on a computing device (e.g., personal computer, desktop computer, laptop computer, reader device, tablet computer, etc.) through a web browser, a web-enabled client, or software residing in non-transitory memory of the computing device. In some embodiments, where the user interface is displayed through a web browser or a web-enabled client, software instructions can be executed on a remote server (or a group of remote servers) which, in turn, can cause the web browser or web-enabled client residing on the computing device to display the user interface. The insights report GUI 1060, 1061 covers a predetermined time period 1062 (e.g., 14 days), and includes a plurality of report portions on a single report GUI, including: a GMI metric 1064, an Ambulatory Glucose Profile (“AGP”) plot 1070, and a glucose control assessment (GCA) 1072. In some embodiments, the insights report GUI 1060, 1061 also includes indicators for high glucose variability 1074. A mathematically-based system and method can be used that exploits the relationship between glucose median, glucose variability, and hypoglycemic risk to prepare a report, and can be implemented in computer software. From this relationship, the insights report 1060, 1061 can be produced. Examining the GMI metric 1064, AGP plot 1070, the GCA table 1072, and the indicators 1074 may provide a good reference during the decision-making process in diabetes treatment. More details can be found in WO 2014/145335 titled “System and Method to Manage Diabetes Based on Glucose Median, Glucose Variability, and Hypoglycemic Risk”, which is hereby expressly incorporated by reference in its entirety for all purposes.

The insights report GUI 1060, 1061 may include one or more GMI metrics 1066, 1068. In one embodiment, insights report GUI 1060, 1061 can include a GMI percentage 1066, a GMI value in mmol/mol 1068, and or both the GMI percentage 1066 and GMI value in mmol/mol 1068 for the time period 1062. Where more than one GMI metric is displayed, one of the GMI metric may be more prominently displayed than the other GMI metric. In another embodiment, the numerical values for the GMI metric may be more prominently displayed than the units. For example, the numerical value of GMI percentage 1064 or GMI value 1068 may displayed in a larger font, and/or in bold or italics, and/or in a different color than the units.

The AGP graph 1070 displays the hourly 5^{th} (1076), 25^{th} (1078), 50^{th} (median) (1080), 75^{th} (1082), and 95^{th} (1084) percentiles of glucose readings, presented over the “typical” day based on all days within the selected timeframe. The AGP plot 1070 may also include two horizontal lines, a “median goal” line 1085 of 154 mg/dL and a low glucose line 1087 of 70 mg/dL.

The first GCA 1072 measure, “Likelihood of Low Glucose” (“LLG”) 1086, is the probability that low glucose values have exceeded an allowable, user-defined threshold. The second measure, “Median Glucose (Compared to Goal)” 1088, is an indication of when the median glucose has exceeded the individual's Median Goal setting. The third measure, “Variability below Median (Median to X^{th} Percentile)” (1090), is a measure of the spread of glucose data below the median. It is calculated as the difference between the 50^{th} and X^{th} percentile glucose readings for the time period. The lower percentile (“X”) may be, e.g., 5%, alternatively 10%, alternatively 15%. It is important to note that when variability below the median is high, it is difficult to achieve the median goal without increasing the Likelihood of Low Glucose (1086). Therefore, factors causing the elevated glucose variability must be addressed before insulin doses are increased, otherwise there would be an increased risk for low glucose. The insights report 1060, 1061 also outlines factors that could contribute to HIGH variability below the median including “Erratic diet,” “Incorrect or missed medication,” “Alcohol consumption,” “Variations in activity level,” or “Illness,” that need to be reviewed and addressed by the health care professional in his/her counseling of the patient. The indicators in the various GCA 1072 categories may be in color, preferably green, yellow, and red, where green indicates a “low” level, yellow indicates a “moderate” level, and red indicates a “high” level of variability, as depicted in FIG. 10F. Alternatively, the indicators may be shown as circles with various levels of shading (no shading (empty), half-circle shaded, or solid), which can correspond to “low” risk (no shading), “medium” risk (half-filled), and “high” risk (solid circle) as seen in FIG. 10E.

The indicators for high glucose variability 1074 include possible factors that could be contributing to glucose variability below the median. Examples include, but are not limited to, erratic diet, incorrect or missed medication, alcohol consumption, variations in activity level, and illness.

Portions of the insights report 1060, 1061, such as the AGP 1070 and GCA 1072, may be divided into time of day periods. The time of day periods can be adjusted according to a patient's particular schedule. The user may set the typical times for Breakfast, Lunch, Dinner, (apple icon) and Bedtime (person in bed icon). These times correspond to daily events that are clinically relevant to diabetes patients whose insulin therapy is related to eating and sleeping events. The result is three daytime periods and two overnight periods, with default time boundaries of 3 am, 8 am, 12 pm, 6 pm, and 10 pm.

FIG. 10G is an example embodiment of GMI metric 1092, as part of glucose profile report GUI 1091, which provides insights regarding the patient's diabetes management. According to one aspect of the embodiments, GUI 1091 can be displayed on a computing device (e.g., personal computer, desktop computer, laptop computer, reader device, tablet computer, etc.) through a web browser, a web-enabled client, or software residing in non-transitory memory of the computing device. In some embodiments, where the user interface is displayed through a web browser or a web-enabled client, software instructions can be executed on a remote server (or a group of remote servers) which, in turn, can cause the web browser or web-enabled client residing on the computing device to display the user interface. According to one aspect of the embodiments, GUI 1091 is a glucose profiles report covering a predetermined time period 1093 (e.g., 14 days), and including a plurality of report portions on a single report GUI, including: a glucose statistics and targets portion 1094 including GMI metric 1092, a Time-in-Ranges portion 1095, an ambulatory glucose profile (AGP) portion 1096, and a daily glucose profiles portion 1097.

The glucose statistics and targets portion 1094 includes the relevant date range, an indication (e.g., percentage) of time that the sensor was active during the specified date range, a list of glucose ranges and targets for patients suffering from diabetes (type 1 or type 2). The targets indicate a % of readings or an amount of time that the patient should target for a particular glucose range for the specified time period. An average glucose level is also reported (either in mg/dL or mmol/L). GMI metric 1092 may also be reported as either a GMI percentage, a GMI value in mmol/mol, or both. A glucose variability percentage may also be reported, which is defined as a percent coefficient of variation.

The Time-in-Ranges portion 1095 depict Time-in-Ranges (also referred to as Time-in-Range and/or Time-in-Target) GUIs, each of which comprise a plurality of bars or bar portions, wherein each bar or bar portion indicates an amount of time that a user's analyte level is within a predefined analyte range correlating with the bar or bar portion. In some embodiments, for example, the amount of time can be expressed as a percentage of a predefined amount of time. Time-in-Ranges GUI portion 1095 includes a single bar comprising five bar portions including (from top to bottom): a first bar portion indicating that the user's glucose range is “Very High” or above 250 mg/dL for 1% (14 minutes) of a predefined amount of time, a second bar portion indicating that the user's glucose range is “High” or between 180 and 250 mg/dL for 18% (4 hours and 19 minutes) of the predefined amount of time, a third bar portion indicating that the user's glucose range is within a “Target Range” or between 70 and 180 mg/dL for 78% (18 hours and 43 minutes) of the predefined amount of time, a fourth bar portion indicating that the user's glucose range is “Low” or between 54 and 69 mg/dL for 3% (43 minutes) of the predefined amount of time, and a fifth bar portion indicating that the user's glucose range is “Very Low” or less than 54 mg/dL for 0% (0 minutes) of the predefined amount of time. As seen in FIG. 10G, according to some embodiments, Time-in-Ranges GUI 1095 can display text adjacent to each bar portion indicating an actual amount of time, e.g., in hours and/or minutes.

According to one aspect of the embodiment shown in FIG. 10G, each bar portion of Time-in-Ranges GUI 1095 can comprise a different color. In some embodiments, bar portions can be separated by dashed or dotted lines and/or interlineated with numeric markers to indicate the ranges reflected by the adjacent bar portions. In some embodiments, the time in ranges reflected by the bar portions can be further expressed as a percentage, an actual amount of time (e.g., 4 hours and 19 minutes), or, as shown in FIG. 10G, both. Furthermore, those of skill in the art will recognize that the percentages of time associated with each bar portion can vary depending on the analyte data of the user. In some embodiments of Time-in-Ranges GUI 1095, the Target Range can be configured by the user. In other embodiments, the Target Range of Time-in-Ranges GUI 1095 is not modifiable by the user.

The glucose profile report GUI 1091 also contains an AGP portion 1096 similar to the AGP graph 1070 described with respect to FIGS. 10E and 10F. The AGP graph displays the hourly 5^{th}, 25^{th}, 50^{th} (median), 75^{th}, and 95^{th} percentiles of glucose readings, presented over the “typical” day based on all days within the selected timeframe. The AGP plot may also include two horizontal lines, which indicate the boundaries of the target range defined in the glucose statistics and targets portion 1094 and the Time-in-Ranges portion 1095. For example, a first line may correspond to the lower boundary of the target range (e.g., 70 mg/dL) and a second line may correspond to an upper boundary of the target range (e.g., 250 mg/dL). The first and second lines may also be color-coded and correspond to the same color as the target range bar portion in the Time-in-Ranges portion 1095 (e.g., green). Thus, the AGP plot of AGP portion 1096 easily illustrates the amount of time spent in (or amount of readings falling within) the target range.

The glucose profile report GUI 1091 may also include a daily glucose profiles section 1297. The daily glucose profiles section 1097 displays a plurality of daily profiles, one for each day of the time period 1093. Each daily profile may represent a midnight to midnight period with the date displayed in the same frame as the profile. In addition to the displaying the date, each profile may also indicate the corresponding day of the week. Each profile may also contain an indication of the target glucose range (e.g., a shaded region or lines indicating the upper and lower boundaries of the target region) to illustrate which parts of each daily profile were within the target range. Portions of the graph outside of the target range may also be color-coded as a further indication of readings or analyte levels that were outside of the target range. The color coding may correspond to the colors used in the Time-in-Ranges 1095 portion. For example, portions of the graph above the target range in the “high” level (e.g., 181-250 mg/dL) may be color-coded yellow. Portions of the graph below the target range in the “low” level (e.g., 54-69 mg/dL) may be color-coded red. The color-coding may consist of coloring the area under the curve a certain color or changing the portion of the graph to be a certain color, or otherwise highlighting the region with the corresponding color.

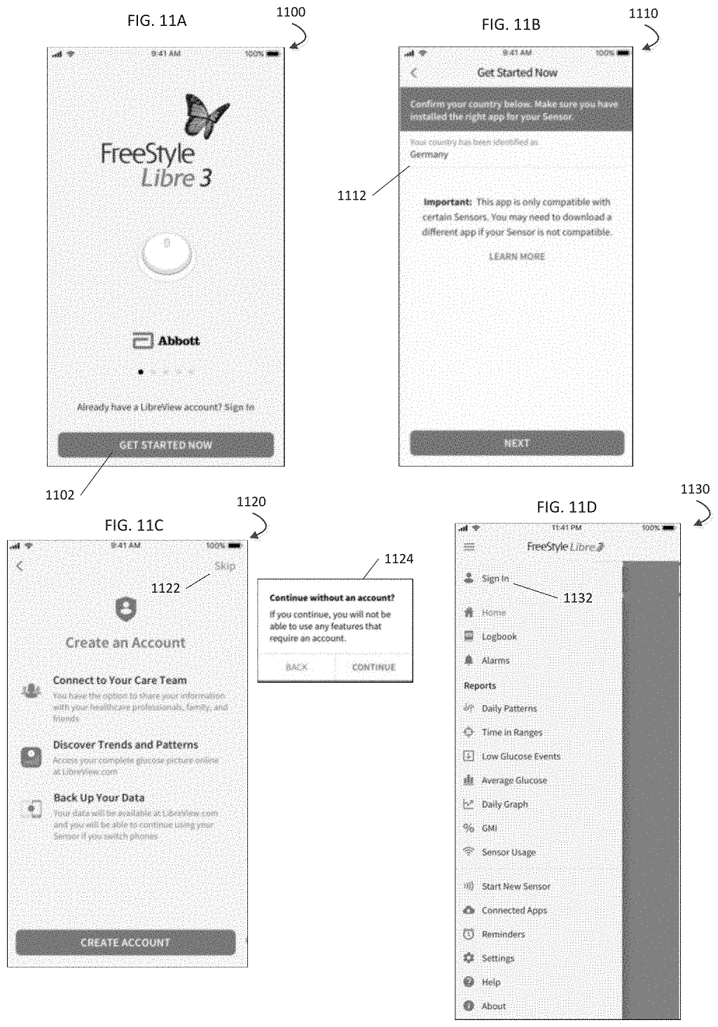

FIGS. 11A to 11D and 12A to 12B depict various GUIs for improving usability and user privacy with respect to analyte monitoring software. FIG. 11A is GUI 1100 depicting a first start interface which can be displayed to a user the first time the analyte monitoring software is started. According to one aspect of the embodiments, GUI 1100 can include a “Get Started Now” button 1102 that, when pressed, will navigate the user to GUI 1110 of FIG. 11B. GUI 1110 depicts a country confirmation interface 1112 that prompts the user to confirm the user's country. According to another aspect of the embodiments, the country selected can limit and/or enable certain interfaces within the analyte monitoring software application for regulatory compliance purposes.

Turning next to FIG. 11C, GUI 1120 depicts a user account creation interface which allows the user to initiate a process to create a cloud-based user account. According to one aspect of the embodiments, a cloud-based user account can allow the user to share information with healthcare professionals, family and friends; utilize a cloud-based reporting platform to review more sophisticated analyte reports; and back up the user's historical sensor readings to a cloud-based server. In some embodiments, GUI 1120 can also include a “Skip” link 1122 that allows a user to utilize the analyte monitoring software application in an “accountless mode” (e.g., without creating or linking to a cloud-based account). Upon selecting the “Skip” link 1122, an information window 1124 can be displayed to inform that certain features are not available in “accountless mode.” Information window 1124 can further prompt the user to return to GUI 1120 or proceed without account creation.

FIG. 11D is GUI 1130 depicting a menu interface displayed within an analyte monitoring software application while the user is in “accountless mode.” According to an aspect of the embodiments, GUI 1130 includes a “Sign in” link 1132 that allows the user to leave “accountless mode” and either create a cloud-based user account or sign-in with an existing cloud-based user account from within the analyte monitoring software application.

Referring next to FIG. 12A, GUI 1240 depicts a research consent interface 1240, which prompts the user to choose to either decline or opt in (through buttons 1242) with respect to permitting the user's sensor readings and/or other product-related data to be used for research purposes.

Referring next to FIG. 12B, GUI 1250 depicts a “Vitamin C” warning interface 1252 which displays a warning to the user that the daily use of more than 500 mg of Vitamin C supplements can result in falsely high sensor readings.

### Example Embodiments of Methods and Systems for Alarm Interfaces

Various example embodiments relating to alarming and alarm suppression methods, alarm interfaces, alarm setup interfaces, compatibility checking interfaces, and alarm logging interfaces for analyte monitoring systems, and other related features will now be described. It will be understood by those of skill in the art that any one or more of the example embodiments of the methods, interfaces, and systems described herein can either be implemented independently, or in combination with any of the other embodiments described in the present application.

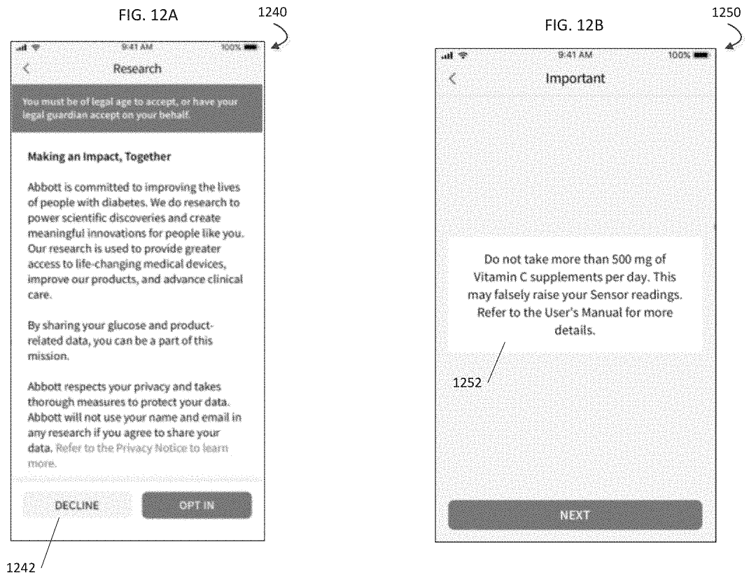

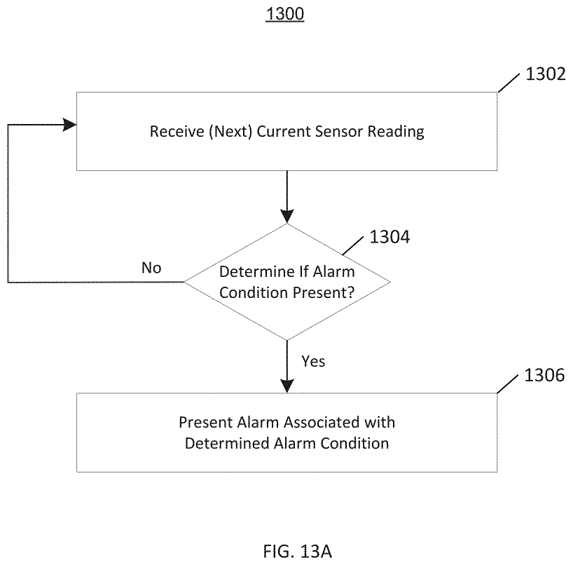

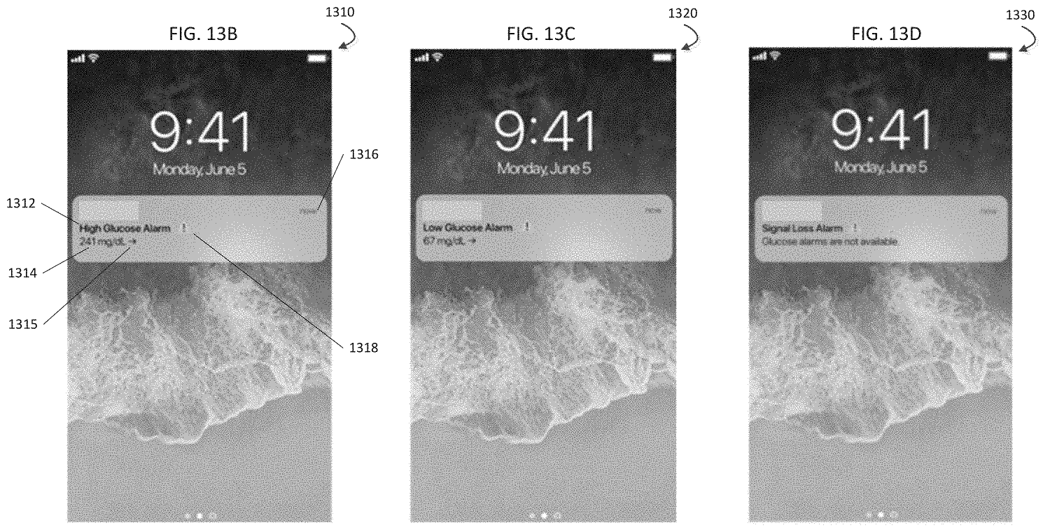

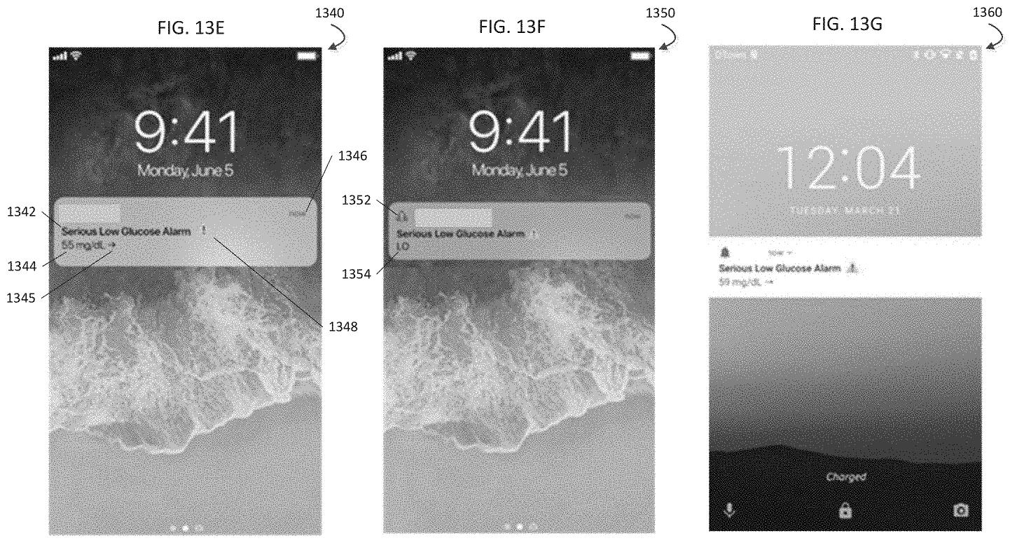

FIG. 13A depicts an example embodiment of a method 1300 for determining one or more alarm conditions and presenting an alarm associated with the determined one or more alarm conditions. At Step 1302, a current sensor reading is received. In some embodiments, the current sensor reading can comprise one or more signals from a glucose sensor disposed in the sensor control device 102, wherein at least a portion of the glucose sensor is configured to be positioned under the subject's skin and in contact with a bodily fluid. In other embodiments, the current sensor reading can be a glucose level measurement received by reader device 120. In some embodiments, the reader device 120 can be in direct communication with sensor control device 102. In other embodiments, reader device 120 can receive a glucose level measurement via another computing device, such as, for example, a cloud-based server. At Step 1304, a determination is made as to whether one or more alarm conditions are present. According to some embodiments, for example, the one or more alarm conditions can comprise at least one of: a low glucose condition, an urgent low glucose condition (also sometimes referred to as a “serious low glucose condition” or a “fixed low glucose condition”), a high glucose condition, a rapidly dropping glucose (rate-of-change) condition, a rapidly rising glucose (rate-of-change) condition, a predicted low glucose condition, or a predicted high glucose condition, amongst other alarm conditions. In some embodiments, the alarm condition can also comprise a signal loss condition, wherein a valid current glucose reading has not been received within a predetermined amount of time (e.g., one minute, five minutes, ten minutes, twenty minutes, etc.). In some embodiments, the signal loss condition can be a result of a loss of wireless connectivity (e.g., Bluetooth connectivity) between reader device 120 and sensor control device 102. Other signal loss conditions and their details are described in the '672 Publication, which is hereby incorporated by reference in its entirety for all purposes. As stated earlier, the determination step can be performed by reader device 120, sensor control device 102, or any another computing device described with respect to analyte monitoring system 100.

Referring still to FIG. 13A, at Step 1306, if it is determined that the one or more alarm conditions are present, an alarm associated with the determined alarm condition is presented. In some embodiments, the presentation of the alarm can comprise a visual notification (e.g., pop-up window, banner notification, full screen notification, etc.). In other embodiments, the presentation of the alarm can comprise a visual notification accompanied by an audible and/or vibratory indicator. In still other embodiments, the presentation of the alarm can comprise an audible and/or vibratory indicator, without a visual notification.

It will be appreciated by those of skill in the art that the method steps described herein can be performed either by a single device or by multiple devices. For example, in some embodiments, the determination of the one or more alarm conditions can be performed by sensor control device 102 and the presentation of alarms can be performed by reader device 120. In other embodiments, both the determination of the alarm condition and the presentation of the alarm can be performed by reader device 120.

FIGS. 13B to 13D are example embodiments of GUIs comprising alarms for use with an analyte monitoring system. FIG. 13B, for example, is a GUI 1310 depicting an alarm for an analyte monitoring system, wherein the alarm comprises an alarm condition text 1312 (e.g., “High Glucose Alarm”), an analyte level measurement 1314 (e.g., a current glucose level of 241 mg/dL) associated with the alarm condition, and a trend indicator 1315 (e.g., a trend arrow or directional arrow) associated with the alarm condition. Additionally, a time-of-alarm indicator 1316 is also shown. In some embodiments, the time-of-alarm indicator 1316 can indicate the amount of time elapsed since the alarm condition was triggered (e.g., now, 5 min ago, 10 min ago). In other embodiments, the time-of-alarm indicator 1316 can indicate the specific time of that the alarm condition was triggered (e.g., 6:12 p.m.). In some embodiments, an alarm icon 1318 can also be adjacent to the alarm condition text 1312.

FIG. 13C is a GUI 1320 depicting another alarm for an analyte monitoring system, wherein the alarm comprises a low glucose alarm for a low glucose alarm condition. FIG. 13D is a GUI 1330 depicting another alarm for an analyte monitoring system, wherein the alarm comprises a signal loss alarm for a signal loss condition. Additional details regarding alarms are described in the '672 Publication, which is hereby incorporated by reference in its entirety for all purposes.

### Example Embodiments of Urgent Low Glucose Alarms

Example embodiments of urgent low glucose alarms will now be described. In a general sense, urgent low glucose alarms for analyte monitoring systems share certain similarities to the alarms previously described with respect to FIGS. 13B to 13D. According to one aspect of the embodiments, urgent low glucose alarms will present an alarm to the user when his or her glucose level has fallen below an urgent low glucose threshold (e.g., below 55 mg/dL). According to another aspect of the embodiments, because of the critical nature of the user's condition, urgent low glucose alarms will override other settings of reader device 120, including the reader device's operating system, such as a “Mute” or “Do Not Disturb” setting. Further, in many embodiments, the glucose threshold levels, which are typically adjustable for other alarms, cannot be changed for urgent low glucose alarms.

FIGS. 13E to 13G are example embodiments of GUIs comprising urgent low glucose alarms for use in an analyte monitoring system. As stated above, these embodiments of alarms have some similarities to the embodiments depicted in FIGS. 13B to 13D. FIG. 13E, for example, is a GUI 1340 depicting an urgent low glucose alarm for an analyte monitoring system, wherein the alarm comprises an urgent low glucose alarm condition text 1342, an analyte level measurement 1344 (e.g., 55 mg/dL) associated with the alarm condition, and a trend indicator 1345 (e.g., a trend arrow or directional arrow) associated with the alarm condition. Additionally, a time-of-alarm indicator 1346 and an alarm icon 1348 are also shown. FIG. 13F is another GUI 1350 depicting an urgent low glucose alarm. As seen in FIG. 13F, a critical alert icon 1352 and an out-of-range (“LO”) text indicator 1354 are displayed in GUI 1350, indicating that the glucose level has dropped below a minimum threshold value within a measurable range. FIG. 13G is another GUI 1360 depicting an urgent low glucose alarm similar to embodiments previously described, but presented through a different mobile operating system (e.g., Android) than the embodiment shown in FIGS. 13E and 13F (e.g., iOS).

According to an aspect of these embodiments, the alarms described herein can include both alarms with configurable settings (e.g., low glucose alarm, high glucose alarm, signal loss alarm) and alarms with non-configurable settings (e.g., urgent low glucose alarm) operating on a single computing device within the same analyte monitoring system. In some embodiments, for example, an analyte monitoring system can include a reader device comprising wireless communication circuitry configured to receive data indicative of an analyte level from a sensor control device, and one or more processors coupled with a memory, the memory storing instructions that, when executed by the one or more processors, cause the one or more processors to: (1) determine whether the data indicative of the analyte level meets one or more alarm conditions, wherein the one or more alarm conditions comprises a first alarm condition associated with a first set of alarms settings that are configurable by the subject, and a second alarm condition associated with a second set of alarm settings that are not configurable by the subject, and wherein the second alarm condition is an urgent low glucose alarm condition; and (2) in response to a determination that at least one of the one or more alarm conditions is met, present an alarm associated with the at least one of the one or more alarm conditions.

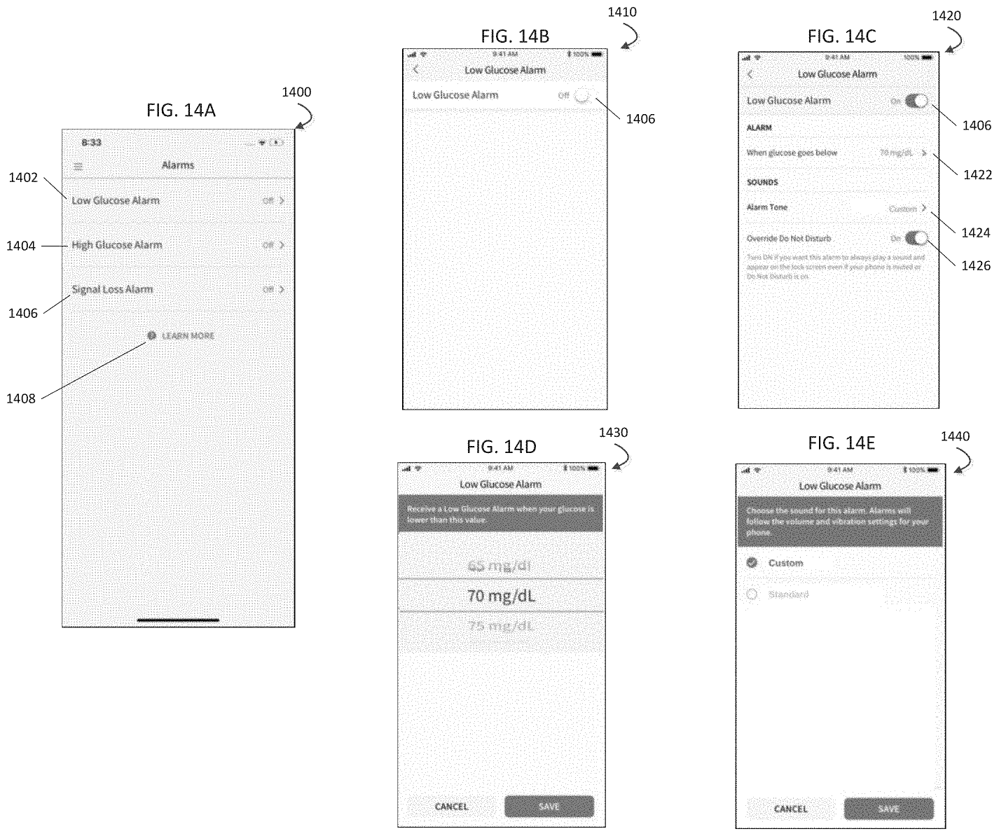

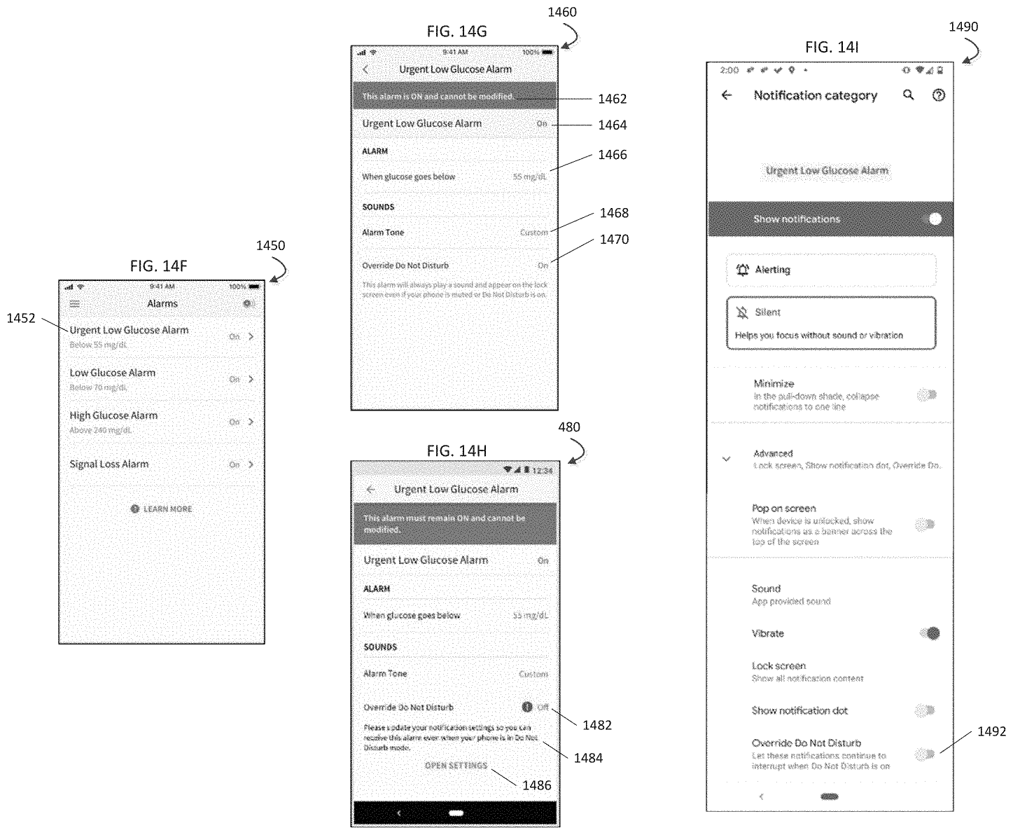

FIGS. 14A to 14E are example embodiments of GUIs including alarm settings for alarms in an analyte monitoring system. FIG. 14A is a GUI 1400 depicting an Alarms settings interface comprising three selectable alarm options: a low glucose alarm option 1402, a high glucose alarm option 1404, and a signal loss alarm option 1406. Adjacent to each selectable alarm option is a textual indicator to indicate whether the alarm is on or off. Additionally, in some embodiments, a selectable “Learn More” option for additional information is provided beneath the selectable alarms. FIG. 14B is a GUI 1410 depicting a Low Glucose Alarms settings interface. According to one aspect of the embodiments, GUI 1410 can be displayed when the user selects the Low Glucose Alarm in previous GUI 1400. According to another aspect of the embodiments, GUI 1410 includes a textual label for the “Low Glucose Alarm” adjacent to a switch 1406 configured to be toggled between an on position and an off position. Those of skill in the art will understand that, instead of a toggle, GUI 1410 can include any one or more of: an on-off checkbox, on-off slider switch, on-off radio buttons, on-off buttons, and the like. As seen in FIG. 14B, when switch 1406 is in the off position, no other settings for the Low Glucose Alarm are available and/or visible.

FIG. 14C is a GUI 1420 depicting the Low Glucose Alarms settings interface after the switch 1406 has been toggled to the on position. After switch 1406 has been toggled to the on position, as can be seen in FIG. 14C, GUI 1420 can include a plurality of configurable settings, including (but not limited to) a low glucose alarm threshold setting 1422, a low glucose alarm tone setting 1424, and a low glucose alarm override setting 1426. According to one aspect of the embodiments, the low glucose alarm threshold setting 1422 can be configured by the user such that the user can select a low glucose threshold (as shown in GUI 1430 of FIG. 14D), where a low glucose alarm will be triggered when the user's glucose level falls below the selected low glucose threshold. According to another aspect of the embodiments, the low glucose alarm tone setting 1424 can be configurable by the user such that the user can select a standard alarm (e.g., adopting the operating system's alarm tone) or a custom low glucose alarm (e.g., allowing the user to select a specific tone or output), as shown in GUI 1440 of FIG. 14E. According to some embodiments, the low glucose alarm tone can be either or both of an audible and a vibratory notification. According to another aspect of the embodiments, the low glucose alarm override setting 1426 can be configured by the user such that, when enabled, a low glucose alarm will always output a sound (or vibration), and display a visual notification on the display of reader device 120 (e.g., on a lock screen), even if reader device 120 is muted or configured in a “Do Not Disturb” mode.

Although FIGS. 14B to 14E depict GUIs having configurable alarm settings for a low glucose alarm, those of skill in the art will recognize that similar GUIs can be utilized for configurable alarm settings for a high glucose alarm or a signal loss alarm, and are fully within the scope of the present disclosure.

FIGS. 14F to 14I are additional example embodiments of GUIs including alarm settings for alarms in an analyte monitoring system. FIG. 14F is a GUI 1450 depicting an Alarms settings interface comprising four selectable alarm options: an urgent low glucose alarm option 1452 (also referred to as a “urgent low glucose alarm” or a “fixed low glucose alarm”), a low glucose alarm option, a high glucose alarm option, and a signal loss alarm option. In one aspect, GUI 1450 is similar to GUI 1400 of FIG. 14A, except that GUI 1450 further includes an urgent low glucose alarm 1452. FIG. 14G is a GUI 1460 depicting an Urgent Low Glucose Alarm settings interface. According to one aspect of the embodiments, GUI 1460 can be displayed when the user selects the Urgent Low Glucose Alarm in previous GUI 1450. According to another aspect of the embodiments, GUI 1460 includes an informational section 1460 that indicates that the Urgent Low Glucose Alarm “is ON and cannot be modified.” In addition, like GUI 1420 of FIG. 14C, GUI 1460 further includes: a textual label for “Urgent Low Glucose Alarm” adjacent to a switch 1464, an urgent low glucose alarm threshold setting 1466, an urgent low glucose alarm tone setting 1468, and an urgent low glucose alarm override setting 1470.

In many embodiments, the aforementioned settings are not configurable to the user, and displayed for informational purposes only. For example, unlike switch 1406 in GUIs 1410 and 1420 (FIGS. 14B and 14C), switch 1464 cannot be toggled to an “Off” position or state. Similarly, according to another aspect of some embodiments, the urgent low glucose alarm threshold setting 1466, the urgent low glucose alarm tone setting 1468, and the urgent low glucose alarm override setting 1470 cannot be modified or disabled.

In other embodiments, one or more of the aforementioned settings may be configurable by the user, while the rest of the settings are displayed for information purposes only. For example, in certain embodiments, the textual label and switch 1464, alarm threshold setting 1466, and alarm override setting 1470 can be non-configurable, while the alarm tone setting 1468 may be configurable so as to allow the user to select a specific tone or vibration. Other combinations of configurable and non-configurable settings are possible, and those of skill in the art will recognize that these combinations are fully within the scope of the present disclosure.

According to another aspect of some embodiments, certain settings may become “active” under certain conditions. FIG. 14H is a GUI 1480 depicting an Urgent Low Glucose Alarm settings interface, similar to GUI 1460 of FIG. 14G. As seen at the bottom third of the interface, the urgent low glucose alarm override setting 1482 is shown in an “Off” state. In addition, a critical alert icon or badge is displayed adjacent to the “Off” state, indicating that corrective action is needed. In some embodiments, further guidance 1484 can be displayed on the interface (e.g., “Please update your notification settings so you can receive this alarm even when your phone is in Do Not Disturb Mode”). According to one aspect of some embodiments, GUI 1480 presents an active “Open Settings” link 1486 near the bottom of the interface. Upon selection of link 1486, GUI 1490 (FIG. 14I) is displayed. In many of the embodiments, GUI 1490 is a notification settings interface provided through the operating system of reader device 120, instead of from within the analyte monitoring software application running on reader device 120. According to another aspect of the embodiments, GUI 1490 includes an “Override Do Not Disturb” setting 1492, which can be enabled by the user. According to an aspect of some embodiments, once the override setting 1492 is enabled, the “Open Settings” link 1486 of GUI 1480 can be transitioned to a hidden or inactive state.

Although the above descriptions of the figures and embodiments refer to alarms and alarm interfaces for a reader device, those of skill in the art will appreciate that these alarms and alarm interfaces can also be implemented in a sensor control device, a local computing system, a trusted computing system, or any other computing device within, or in communication with, an analyte monitoring system. Moreover, as described earlier, any of the GUIs and features described herein can comprise instructions stored in memory of a reader device, sensor control device, or any other computing device that is part of, or in communication with, the analyte monitoring system.

### Example Embodiments of Alarm Suppression Features

Example embodiments of methods and systems for alarm suppression features in an analyte monitoring system will now be described. Generally, without the ability to moderate alarms in an analyte monitoring system, adverse effects ranging from mild irritation to de-sensitization can arise. In particular, these effects can lead to dangerous consequences, such as a user disregarding or ignoring critical alarms of the analyte monitoring system. Thus, there exists a need for robust methods and systems for alarm suppression features in an analyte monitoring system.

As stated earlier, those of skill in the art will recognize that the method steps described herein can comprise instructions (e.g., software, firmware, etc.) stored in non-transitory memory of sensor control device 102, reader device 120, or any other computing device or system that is part of, or in communication with, analyte monitoring system 100. Further, the method steps described herein can be performed by a single centralized device or by multiple devices.

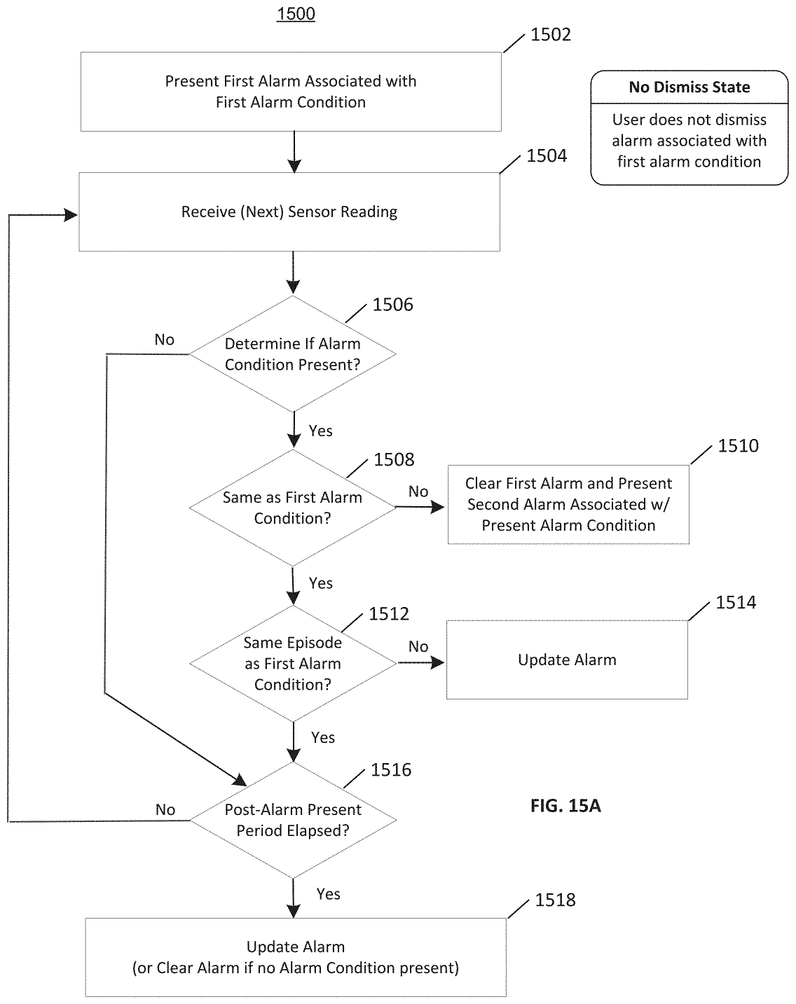

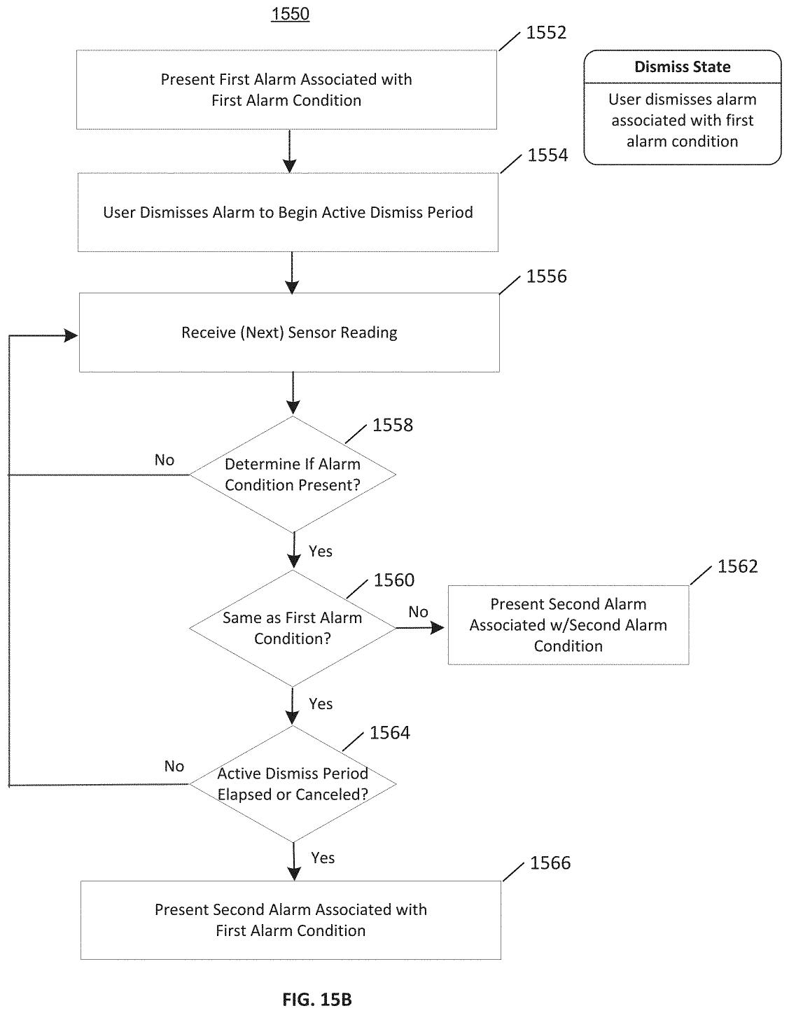

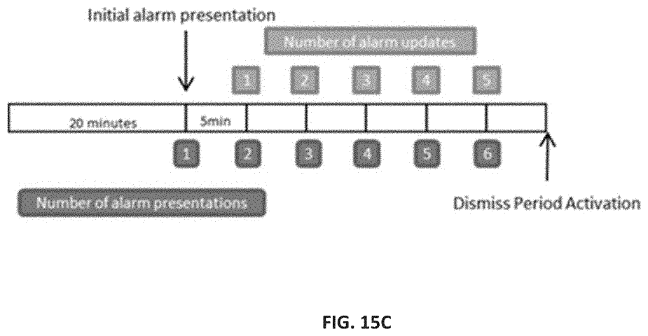

FIG. 15A is a flow diagram depicting an example embodiment of a method 1500 for suppressing alarms during a post-alarm presentation period in an analyte monitoring system. According to one aspect of the embodiments, a post-alarm presentation period comprises a predetermined amount of time after an alarm has been presented, during which the same alarm will not be repeated in response to the same alarm condition. In many embodiments, the post-alarm presentation period can be the same for all alarms. In other embodiments, the post-alarm presentation period can be configured for each alarm. Furthermore, method 1500 assumes that the user has not dismissed or otherwise taken any action in response to the alarm.

At Step 1502, a first alarm is presented, wherein the first alarm is associated with a first alarm condition. In some embodiments, the first alarm condition can be determined using method 1300, as described with respect to FIG. 13A, and the presentation of the first alarm can comprise any of the interfaces described with respect to FIGS. 13B to 13G. At Step 1504, a sensor reading is received. In some embodiments, the sensor reading can comprise a current glucose value. In other embodiments, the sensor reading can comprise a historical glucose value. Thereafter, at Step 1506, a determination is made as to whether an alarm condition is present with respect to the received sensor reading (or, in the case of a signal loss alarm condition, a lack thereof). If an alarm condition is not present, then a determination is made, at Step 1516, as to whether a post-alarm presentation period has elapsed. According to some embodiments, the post-alarm presentation period can be a predetermined amount of time (e.g., 1, 2, 5, 10, 15 minutes, etc.). In other embodiments, the post-alarm presentation period can be based on a count value that is incremented. At Step 1516, if the post-alarm presentation period has not elapsed, then no further action is taken with respect to the first alarm and method 1500 returns to Step 1504, and the next sensor reading is received. However, if the post-alarm presentation period has elapsed, then at Step 1518, the presentation of the first alarm is cleared. In some embodiments, the first alarm is cleared when a visual notification is removed from the display. In other embodiments, the first alarm is cleared when an audible tone or vibration is stopped, or the repetition of an audible tone or vibration is discontinued.

Returning to Step 1506, if a determination is made that an alarm condition is present, then at Step 1508, it is then determined whether the present alarm condition is the same as the first alarm condition. If the present alarm condition is determined to be a different alarm condition than the first alarm condition then, at Step 1510, the first alarm is cleared and a second alarm associated with the present (e.g., second) alarm condition is presented.

For illustration, and without the intent to limit the embodiments, if the first alarm presented is a low glucose alarm associated with a low glucose condition at Step 1502, and an alarm condition is subsequently determined to be a high glucose condition at Steps 1504, 1506, and 1508, then, at Step 1510, the low glucose alarm is cleared and a high glucose alarm is presented.

Returning to Step 1508, if a determination is made that the present alarm condition is the same as the first alarm condition then, at Step 1512, it is subsequently determined whether the present alarm condition is part of the same episode as the first alarm condition. If it is determined that the present alarm condition is not part of the same episode as the first alarm condition then, at Step 1514, the alarm is updated. According to some embodiments, the step of updating the alarm can comprise presenting a new notification interface (e.g., a new analyte level measurement 1314, as shown in FIG. 13B). In some embodiments, the new notification interface can completely replace the previous notification interface. In other embodiments, the new notification interface can be stacked on top of the previous notification interface.

For illustration, and without the intent to limit the embodiments, if the first alarm presented is a high glucose alarm associated with a high glucose condition (e.g., 241 mg/dL) at Step 1502, and the present alarm condition is subsequently determined to also be a high glucose condition (e.g., 255 mg/dL) at Steps 1504, 1506, and 1508, but the high glucose condition is determined to be part of a different episode from the first high glucose condition at Step 1512, then the high glucose alarm is updated at Step 1514 (e.g., a new notification interface indicating an analyte measurement of 255 mg/dL replaces the previous notification interface).

Returning to Step 1512, if a determination is made that the present alarm condition is part of the same episode as the first alarm condition then, at Step 1516, it is then determined whether the post-alarm presentation period has elapsed. If the post-alarm presentation period has elapsed, then the alarm is updated at Step 1518. If the post-alarm presentation period has not elapsed, then no action is taken with respect to the first alarm, and method 1500 returns to Step 1504 to receive the next sensor reading.

With reference to Step 1512 of method 1500, according to one aspect of the embodiments, to determine whether the present (e.g., second) alarm condition is part of the same episode as the previous (e.g., first) alarm condition, certain criteria can be evaluated. For example, in some embodiments, a determination can be made that a high glucose episode has ended when:

Sensor reading<HG_{threshold}−HG_{tolerance} (1)

In Equation (1) above, the sensor reading can be a current glucose value or a historical glucose value, HG_{threshold} is a high glucose threshold, and HG_{tolerance} is a high glucose threshold tolerance. Furthermore, in some embodiments, HG_{tolerance} can equal F_HIGH*HG_{threshold}+C_HIGH, wherein F_HIGH is a positive fraction up to one place after the decimal and C_HIGH is a scalar factor.

As another example, in some embodiments, a determination can be made that a low glucose episode has ended when:

Sensor reading<LG_{threshold}+LG_{tolerance} (2)

In Equation (2) above, LG_{threshold} is a low glucose threshold and LG_{tolerance} is a low glucose threshold tolerance.

As another example, in some embodiments, a determination can be made that an urgent low glucose episode has ended when:

Sensor reading>ULG_{threshold}+ULG_{tolerance} (3)

In Equation (3) above, ULG_{threshold} is an urgent low glucose threshold and ULG_{tolerance} is an urgent low glucose threshold tolerance.

Those of skill in the art will appreciate that other methods for identifying analyte episodes can also be used in addition to, or in lieu of, the above equations. Further descriptions of such methods are described, for example, in U.S. Pat. No. 9,622,689, U.S. Patent Appl. Publ. Nos. 2018/0226150 and 2018/0217917, all of which are hereby incorporated by reference in their entireties for all purposes.

Furthermore, it will be understood by those of skill in the art that any of the above steps, or combinations of steps, can be optional to method 1500. For example, according to some embodiments, Steps 1512 and 1514, which relate to determining whether the present alarm condition is part of the same episode as the first alarm condition can be optional, and thus Step 1508 would proceed to Step 1516.

FIG. 15B is a flow diagram depicting an example embodiment of a method 1550 for suppressing alarms during an active dismiss period in an analyte monitoring system. According to one aspect of the embodiments, when a user dismisses an alarm, the alarm's visual and/or audible presentation stops and a dismiss period is activated during which recurring conditions for the same alarm are not triggered. An active dismiss period comprises a predetermined amount of time after the alarm has been dismissed by the user. In many embodiments, the active dismiss period can be the same for all alarms. In other embodiments, the active dismiss period can be configured to be customized for one or more alarms. Those of skill in the art will also recognize that any combination, subset, or all of the steps of method 1550 can be implemented in combination with any combination, subset, or all of the steps of method 1500 described above.

At Step 1552, a first alarm is presented, wherein the first alarm is associated with a first alarm condition. In some embodiments, the first alarm condition can be determined using method 1300, as described with respect to FIG. 13A, and the presentation of the first alarm can comprise any of the interfaces described with respect to FIGS. 13B to 13G. At Step 1554, the user dismisses the presented alarm to begin an active dismiss period. According to some embodiments, the active dismiss period can be a predetermined amount of time (e.g., 5, 10, 15, 20, 30 minutes, etc.). In other embodiments, the active dismiss period can be based on a count value that is incremented or a countdown timer.

At Step 1556, a sensor reading is received. In some embodiments, the sensor reading can comprise a current glucose value. In other embodiments, the sensor reading can comprise a historical glucose value. Thereafter, at Step 1558, a determination is made as to whether an alarm condition is present with respect to the received sensor reading (or, in the case of a signal loss alarm condition, a lack thereof). If an alarm condition is not present, then method 1550 returns to Step 1556 to receive a next sensor reading. If an alarm condition is present then, at Step 1560, a determination is made as to whether the present (e.g., second) alarm condition is the same as the previous (e.g., first) alarm condition. If the present (e.g., second) alarm condition (e.g., high glucose condition) is different from the previous (e.g., first) alarm condition (e.g., low glucose condition), then at Step 1562, the first alarm (e.g., low glucose alarm) is cleared and a second alarm (e.g., high glucose alarm) associated with the second alarm condition (e.g., high glucose condition) is presented. In some embodiments, an alarm is cleared when a visual notification is removed from the display. In other embodiments, an alarm is cleared when an audible tone or vibration is stopped, or the repetition of an audible tone or vibration is discontinued.

Returning to Step 1560, if it is determined that the present (e.g., second) condition is the same as the previous (e.g., first) condition then, at Step 1564, a determination is then made as to whether the active dismiss period has either elapsed or been canceled. If the active dismiss period has either elapsed or been canceled then, at Step 1566, a second alarm associated with the alarm condition is presented. If the active dismiss period has neither elapsed nor been canceled, then no further action is taken (e.g., no second alarm is presented), and method 1550 returns to Step 1556 to receive the next sensor reading. In one aspect, the active dismiss period thereby prevents a second alarm from being triggered too soon after a first alarm that was based on the same alarm condition, after a user has dismissed the first alarm.

According to one aspect of the embodiments, the active dismiss period has elapsed when a predetermined amount of time since the user dismissed the first alarm has passed. The active dismiss period can range, for example, from five minutes to 1440 minutes. Those of skill in the art will recognize that other measures of time for the active dismiss period can be implemented as well (e.g., timer, counter, etc.), and that those values are within the scope of the present disclosure.

According to another aspect of the embodiments, the active dismiss period can also be canceled (before the predetermined amount of time has elapsed) by one or more predefined events and/or conditions: an alarm threshold setting changed, an alarm disabled, a glucose episode ended, a sensor ended (e.g., sensor control device entering an end-of-life state), a sensor terminated, a new sensor started, and/or a device reset. Those of skill in the art will recognize that other events and/or conditions can be utilized to cancel the active dismiss period, and that those events and/or conditions are within the scope of the present disclosure.

Additionally, certain types of alarms can be configured with special active dismiss period conditions. For example, according to some embodiments, a signal loss alarm may be configured such that the active dismiss period is not activated until there are a predetermined number of consecutive signal loss alarm presentations. The signal loss alarm can also be dismissed automatically after the predetermined number of consecutive signal loss alarm presentations. For illustration, as shown in FIG. 15C, the signal loss alarm can be configured such that when there are six consecutive presentations for a thirty minute period, the active dismiss period is automatically activated without user intervention. Although the above example is provided for a signal loss alarm, those of skill in the art will recognize that these embodiments can be applied to other types of alarms as well.

### Example Embodiments of Alarm Setup Interfaces

Example embodiments of methods and systems for alarm setup GUIs in an analyte monitoring system will now be described. As previously described with respect to FIGS. 14H and 14I, certain alarms can require that the user configure specific operating system features that may interfere with the alarms. For example, some operating systems for mobile computing devices include such features as “Do Not Disturb” or “Mute” that may interfere with the audible, visual, or vibratory alarm presentation of the aforementioned alarms. While these operating system features may be disabled on a system-wide basis or customizable for each application, the actual process to configure these features correctly can be difficult for the user and/or subject to error. Thus, it would be advantageous to include easy-to-use alarm setup GUIs for the user to mitigate the risk of interference of these operating system features with the alarms.

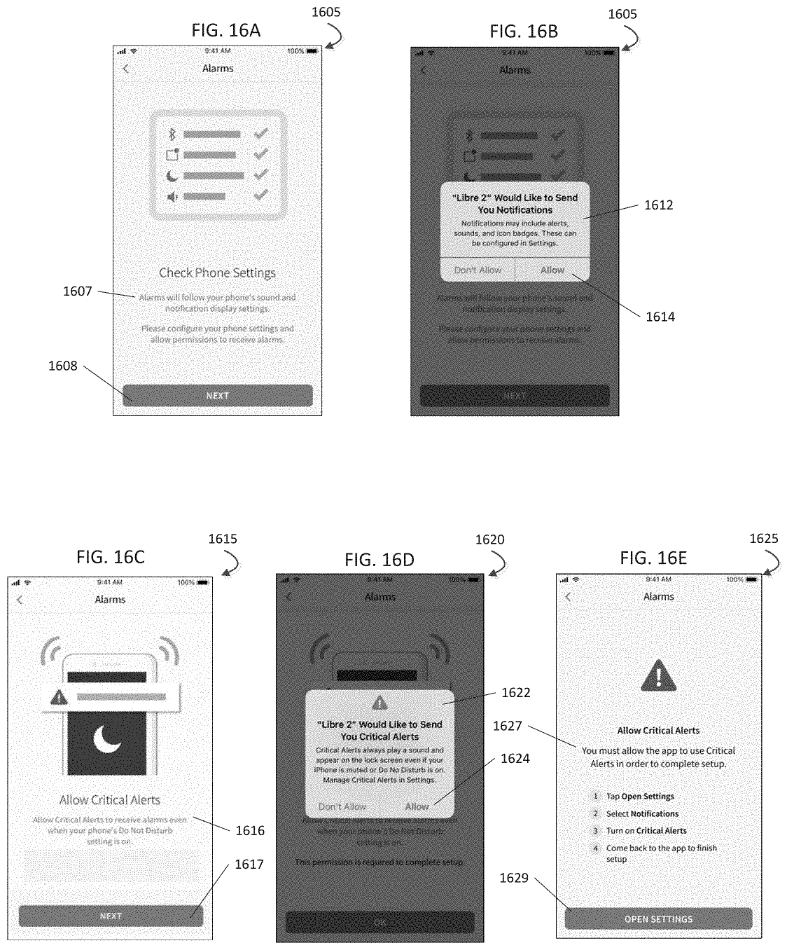

FIGS. 16A to 16E depict example embodiments of alarm setup interfaces in an analyte monitoring system. FIG. 16A is a GUI 1605 depicting an Alarms setup interface providing instructions 1607 to the user to configure certain system settings and permissions of reader device 120 in order to allow for alarms. In some embodiments, GUI 1605 further includes a button or link 1608 that, when pressed by the user, causes reader device 120 to display a notification permissions interface 1612, as shown in FIG. 16B. According to some embodiments, the notification permissions interface 1612 can include an “Allow” button or link 1614 to allow the analyte monitoring software application to send notifications to reader device 120.

Referring next to FIG. 16C, GUI 1615 depicts another Alarms setup interface providing instructions 1616 to the user to configure certain system settings to allow critical alerts to receive alarm even when reader device 120 is in a “Do Not Disturb” mode. In some embodiments, GUI 1615 further includes a button or link 1617 that, when pressed by the user, causes reader device 120 to display a critical alerts permission interface 1622, as shown in FIG. 16D. According to some embodiments, the critical alerts permission interface 1622 can include an “Allow” button or link 1624 to allow the analyte monitoring software application to send critical alerts to reader device 120.

According to one aspect of some embodiments, if the user did not enable either or both of: (1) notification permissions (FIG. 16B), and (2) critical alerts permissions (FIG. 16D), then another interface, shown in FIG. 16E, is displayed to the user. In particular, FIG. 16E is a GUI 1625 depicting another Alarms setup interface providing instructions 1627 to the user about how to manually set up the notification permissions and critical alerts settings for use with the analyte monitoring software application. According to some embodiments, if it is determined that the user did not enable one or more required alarm permissions settings, then the analyte monitoring software application can remain in an inoperable or partially operable state until the required alarm permissions settings are enabled. In this regard, the user is otherwise prevented from further operating the analyte monitoring software application while a predetermined set of alarm permissions settings are not enabled. Likewise, in some embodiments, if it is determined that the user has later disabled one or more required alarm permissions settings, the user can be prompted with an interface to manually correct the alarm permissions settings and, furthermore, prevented from further operating the analyte monitoring software application. In this regard, the analyte monitoring software application is able to mitigate the risk of a user not receiving critical alerts and other alarms for adverse conditions.

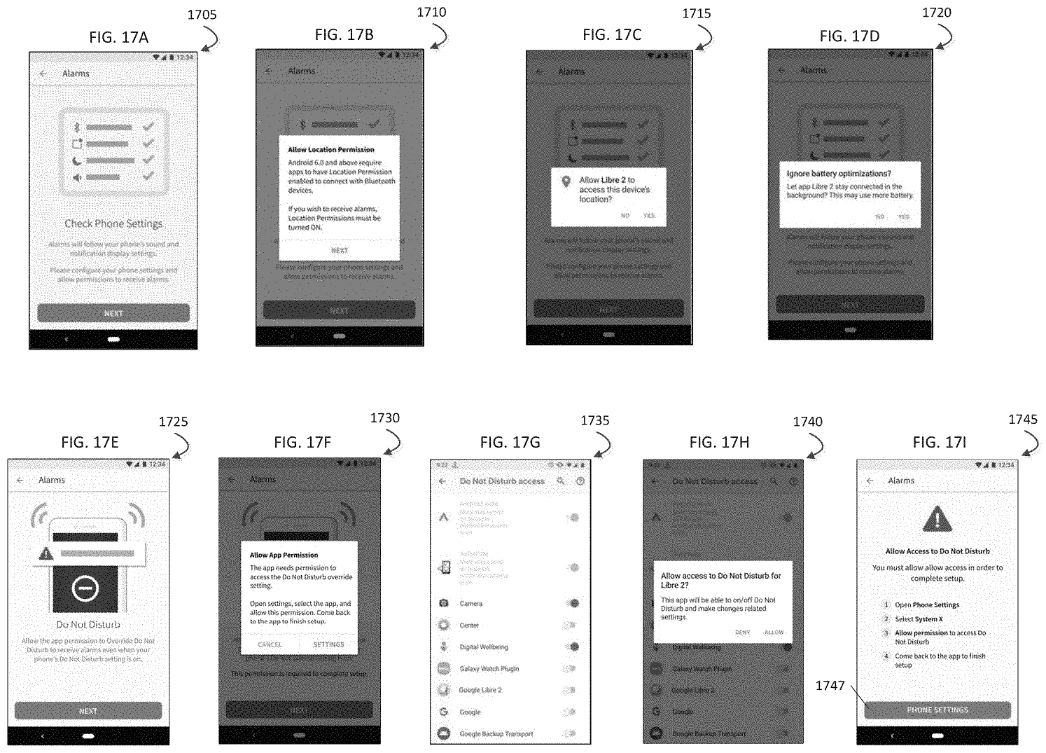

FIGS. 17A to 17I depict additional example embodiments of alarm setup interfaces in an analyte monitoring system. Generally, FIGS. 17A to 17I share many similarities to the alarm setup interfaces depicted in FIGS. 16A to 16E. However, the interfaces shown in FIGS. 17A to 17I are directed at a different operating system and includes different alarm permissions settings. For example, GUIs 1710 and 1715 of FIGS. 17B and 17C are directed to enabling location permissions. GUI 1720 of FIG. 17D is directed to interfaces for enabling a system setting to ignore battery optimization settings (e.g., so as not to disable alarms during low power conditions). GUIs 1725, 1730, 1735, and 1740 (e.g., FIGS. 17E to 17H) are directed to interfaces to allow the analyte monitoring software application to override the “Do Not Disturb” features in the operating system.

According to one aspect of some embodiments, if the user did not enable one or more of the aforementioned alarm permissions settings, then another interfaces, shown in FIG. 17I, is displayed to the user. Similar to the embodiments described with respect to FIG. 16E, FIG. 17I is a GUI 1745 depicting another Alarms setup interface providing instructions 1747 to the user about how to manually configure system settings for use with the analyte monitoring software application. According to some embodiments, if it is determined that the user did not enable one or more required alarm permissions settings, then the user is prevented from further operating the analyte monitoring software application. In addition, in some embodiments, if it is determined that the user has disabled one or more required alarm permissions settings, the user can be prompted with an interface to manually correct the alarm permissions settings and, furthermore, prevented from further operating the analyte monitoring software application. In this regard, the analyte monitoring software application is able to mitigate the risk of a user not receiving critical alerts and other alarms for adverse conditions.

### Example Embodiments of Alarm Unavailability Features and Interfaces

Example embodiments of methods, systems, and related GUIs for detecting the unavailability of alarms in an analyte monitoring system will now be described. As previously described, certain operating system features (e.g., power optimization features, “Do Not Disturb” features, etc.) can interfere with alarms in an analyte monitoring system. In addition, users can unknowingly take certain actions with their analyte monitoring system that can also interfere with alarms. Thus, needs exist for robust methods and system for detecting the unavailability of alarms in an analyte monitoring system.

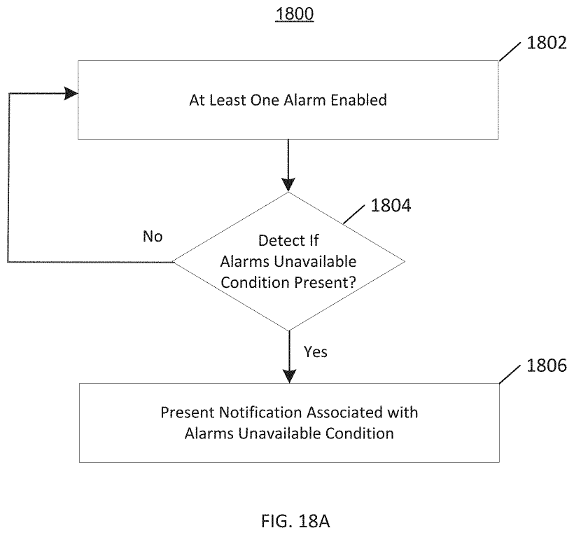

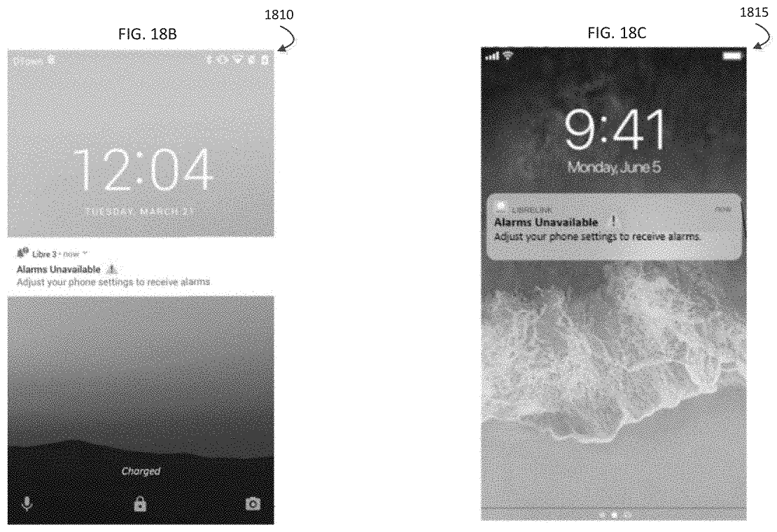

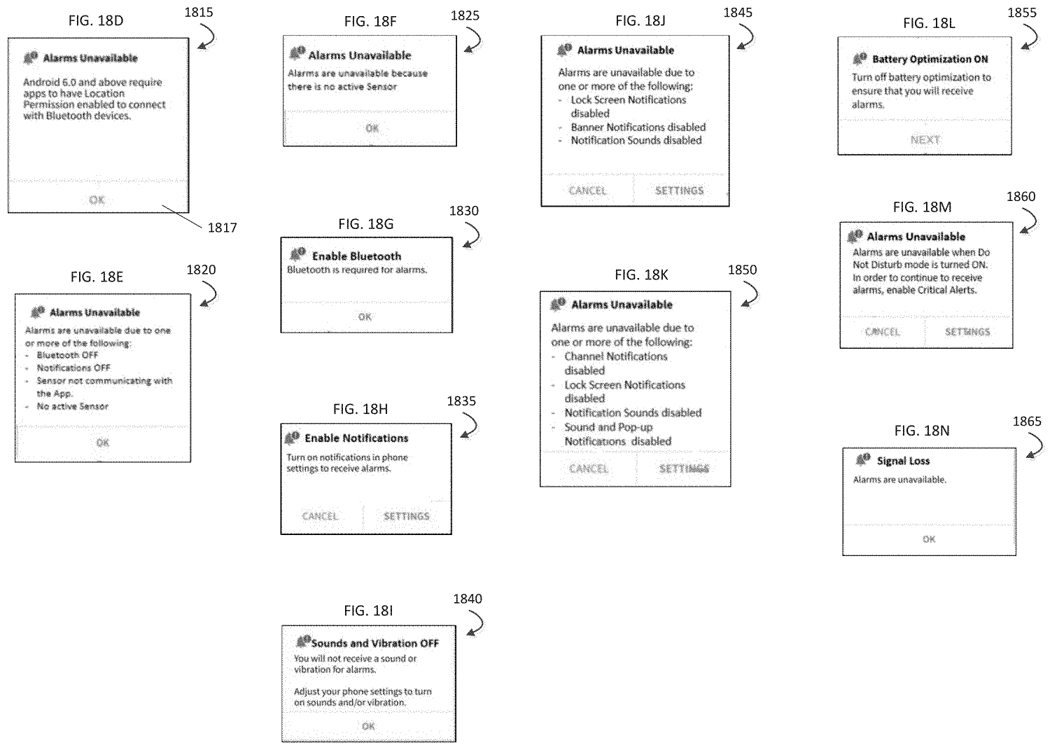

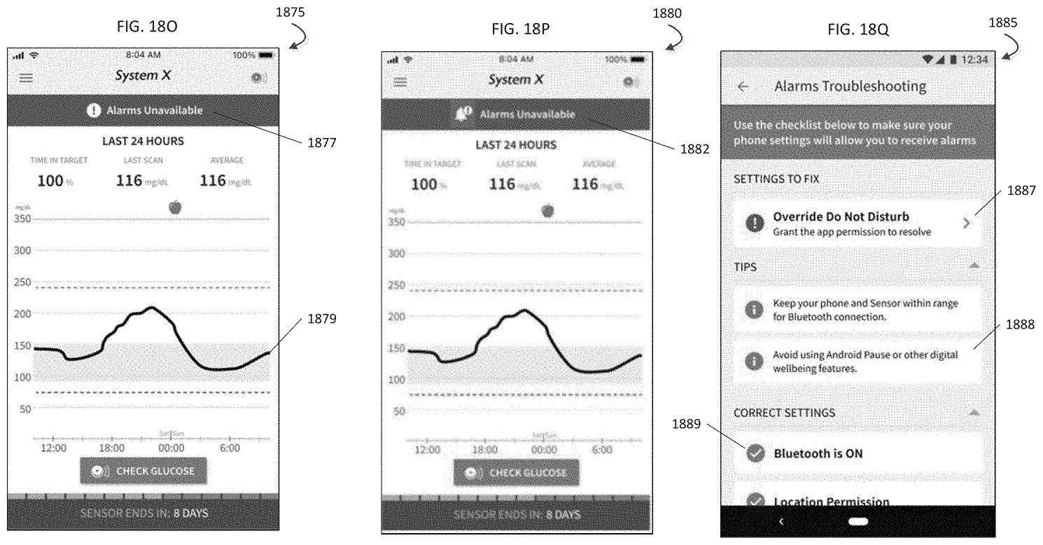

FIG. 18A depicts an example embodiment of a method for determining an alarm unavailability condition is present in an analyte monitoring system. At Step 1802, one or more alarms are enabled in an analyte monitoring system. In many embodiments, the one or more alarms can comprise a low glucose alarm, an urgent low glucose alarm, a high glucose alarm, and/or a signal loss alarm, amongst other examples, that can be enabled on one or more of: a reader device 120, a sensor control device 102, or any other computing device that is a part of, or in communication with, the analyte monitoring system.

At Step 1804, a determination is made as to whether one or more alarm unavailability conditions are present. According to one aspect of the embodiments, alarm unavailability conditions can comprise any one or more of the following conditions: the wireless communication circuitry (e.g., Bluetooth or Bluetooth Low Energy) is disabled and/or not functioning, systemwide notifications are disabled, application-specific notifications are disabled, a mute or silent feature is enabled, the analyte monitoring software application has been force-closed by the user or by the system (i.e., no longer running in the background or foreground), critical alerts are disabled, the “Override Do Not Disturb” feature is disabled, the “Do Not Disturb” channel is turned off; alarm tone(s) are set to silent, location permissions are disabled, and/or the battery optimization feature(s) are enabled. In some embodiments, other alarm unavailability conditions can further include: no active sensor detected or sensor fault conditions (e.g., temperature too high, temperature too low, sensor not communicating with reader device 120). Those of skill in the art will recognize that these aforementioned alarm unavailability conditions are meant to be illustrative only, and do not represent an exhaustive list of all alarm unavailability conditions. Other conditions relating to either the analyte sensor, sensor control device 102, or reader device 120 that can cause interference with either: (1) the determination of alarm conditions, or (2) the presentation of alarms in the analyte monitoring system are possible and are fully within the scope of the present disclosure.

Referring again to FIG. 18A, if no alarm unavailability conditions are detected, then method 1800 returns to Step 1802 and continues to monitor for alarm unavailability conditions (as long as at least one alarm is enabled). However, if one or more alarm unavailability conditions are detected, then, at Step 1806, one or more notifications associated with the detected one or more alarms unavailability conditions are presented to the user.

According to one aspect of the embodiments, the one or more notifications associated with the detected one or more alarms unavailability conditions can comprise banner notifications or pop-up windows displayed to the user outside of the analyte monitoring software application (e.g., on a lock screen), as seen in GUI 1810 (FIG. 18B) and GUI 1815 (FIG. 18C).

According to another aspect of the embodiments, the one or more notifications associated with the detected one or more alarm unavailability conditions can comprise modal windows, as seen in GUIs 1815 to 1865 (FIGS. 18D to 18N). In some embodiments, the modal can provide information regarding the specific cause of the alarm unavailability condition, along with a confirmation (“OK”) button, as seen in FIG. 18D (location permission not enabled), FIG. 18F (no active sensor), and FIG. 18G (Bluetooth disabled). In some embodiments, the modal can provide a number of possible reasons for the alarm unavailability condition along with a confirmation (“OK”) button, as seen in FIG. 18E. In other embodiments, the modal can provide a number of possible reasons for the alarm unavailability condition along with a “Settings” button that opens up the corresponding settings interface to allow the user to correct the condition, as seen in FIGS. 18J and 18K. In still other embodiments, the modal can present a specific alarm that is unavailable, the reason for the alarm unavailability condition, and a “Settings” button to that opens up the corresponding settings interface to allow the user to correct the condition. These examples are meant to be illustrative only, and those of skill in the art will recognize that other combinations and permutations of modals can be implemented and are fully within the scope of the present disclosure.

According to another aspect of the embodiments, the one or more notifications associated with the detected one or more alarm unavailability conditions can comprise an in-app notification within an analyte monitoring software application, as seen in GUI 1875 and GUI 1880 (FIGS. 18O and 18P). In some embodiments, the notification associated with the alarm unavailability condition can be presented as an in-app banner notification 1877 positioned on a same interface as an analyte trend graph 1879. Although not shown, in some embodiments, the in-app banner notification 1877 can persist through different interfaces within the analyte monitoring software application (e.g., reports, logbook, etc.). In this regard, the in-app banner notification 1877 allows for the user to continue to review recent and historical analyte data, as well as reports. According to some embodiments, the banner notification can also be configured to be an active link such that when it is pressed by the user, an alarm troubleshooting interface 1885 is displayed. In some embodiments, the alarm troubleshooting interface 1885 can further include one or more active links 1887 to specific system settings that can be changed. In some embodiments, the alarm troubleshooting interface 1885 can also include informational tips 1888 to resolve one or more alarm unavailability conditions. Furthermore, in some embodiments, the alarm troubleshooting interface 1885 can also include a correct settings section 1889 to indicate settings that are correctly configured. In this regard, the in-app notification can be configured to prompt the user to take corrective action regarding the alarms unavailable condition.

### Example Embodiments of Alarm Logging Features

Example embodiments of methods, systems, and related GUIs for logging alarms in an analyte monitoring system will now be described. Generally, many of the alarms described herein can provide timely and important information to a user of an analyte monitoring system. However, these alarms are typically intended to convey current or recent information. It would also be advantageous for the user of an analyte monitoring system to be able to review alarm events and their corresponding conditions retrospectively.

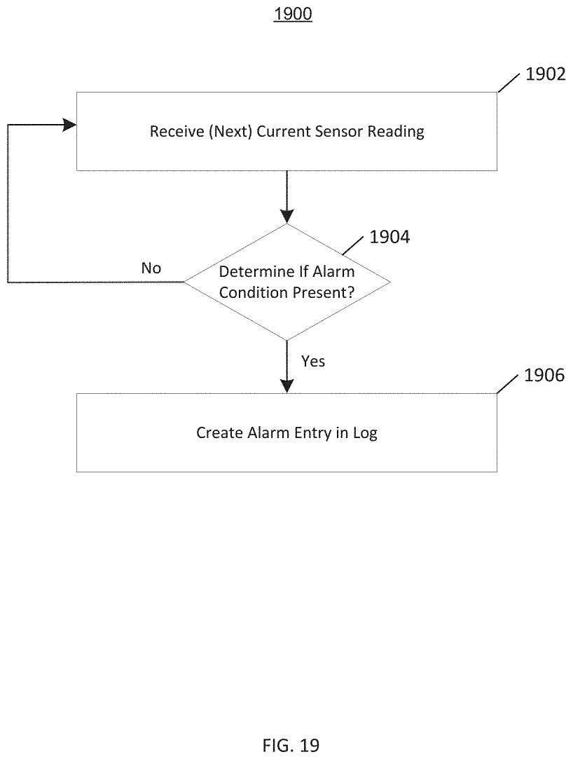

FIG. 19 depicts an example embodiment of a method 1900 for determining one or more alarm conditions and logging an alarm associated with the determined one or more alarm conditions. At Step 1902, a current sensor reading is received. In some embodiments, the current sensor reading can comprise one or more signals from a glucose sensor disposed in the sensor control device 102, wherein at least a portion of the glucose sensor is configured to be positioned under the subject's skin and in contact with a bodily fluid. In other embodiments, the current sensor reading can be a glucose level measurement received by reader device 120. In some embodiments, the reader device 120 can be in direct communication with sensor control device 102. In other embodiments, reader device 120 can receive a glucose level measurement via another computing device, such as, for example, a cloud-based server.

At Step 1904, a determination is made as to whether one or more alarm conditions are present. According to some embodiments, for example, the one or more alarm conditions can comprise at least one of: a low glucose condition, an urgent low glucose condition (also sometimes referred to as an “urgent low glucose condition” or a “fixed low glucose condition”), a high glucose condition, a rapidly dropping glucose (rate-of-change) condition, a rapidly rising glucose (rate-of-change) condition, a predicted low glucose condition, or a predicted high glucose condition, amongst other alarm conditions. In some embodiments, the alarm condition can also comprise a signal loss condition, wherein a valid current sensor reading has not been received within a predetermined amount of time (e.g., one minute, five minutes, ten minutes, twenty minutes, etc.). In some embodiments, the signal loss condition can be a result of a loss of wireless connectivity (e.g., Bluetooth connectivity) between reader device 120 and sensor control device 102. Other signal loss conditions and their details are described in the '672 Publication, which is hereby incorporated by reference in its entirety for all purposes. As stated earlier, the determination step can be performed by reader device 120, sensor control device 102, or any another computing device described with respect to analyte monitoring system 100.

Referring still to FIG. 19, at Step 1906, if it is determined that one or more alarm conditions are present, an alarm entry is created in a log. In some embodiments, creation of the log entry can occur prior to the presentation of the alarm (e.g., pop-up window, banner notification, full screen notification, audible indicator, vibratory indicator, etc.). In other embodiments, the creation of the log entry can occur after the presentation of the alarm. In still other embodiments, the creation of the log entry can occur upon or after dismissal of the alarm presentation by the user.

According to one aspect of the embodiments, the log can be a discrete file on any one of more of: sensor control device 102, reader device 120, or any other computing device that is a part of, or in communication with, analyte monitoring system 100. In some embodiments, for example, the log can be a part of the analyte monitoring software application residing in memory of reader device 120.

It will also be appreciated by those of skill in the art that the method steps described herein can be performed either by a single device or by multiple devices. For example, in some embodiments, the determination of the one or more alarm conditions can be performed by sensor control device 102 and the presentation of alarms can be performed by reader device 120. In other embodiments, both the determination of the alarm condition and the presentation of the alarm can be performed by reader device 120.

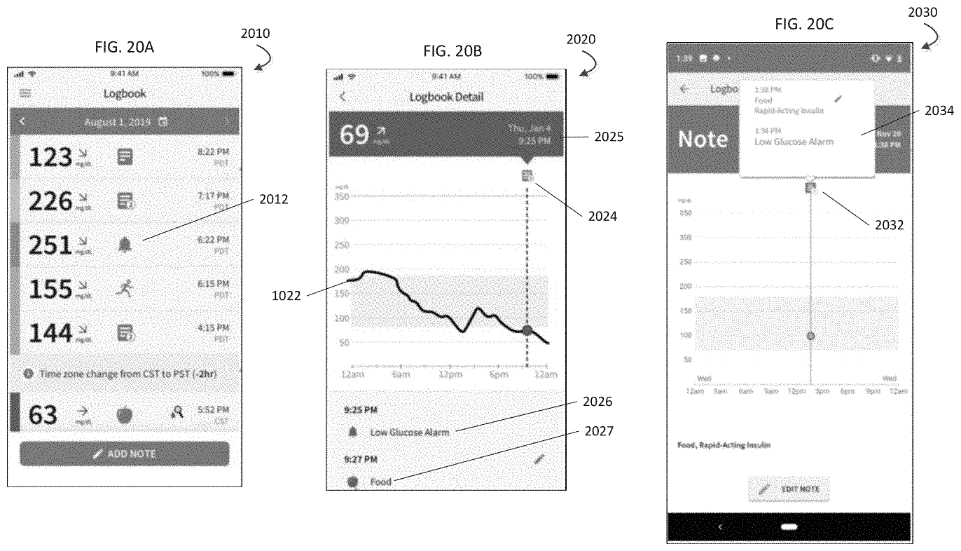

FIGS. 20A to 20C depict example embodiments of alarm logging interfaces for an analyte monitoring system. FIG. 20A is a GUI 2010 depicting a logbook interface for an analyte monitoring software application installed on reader device 120. According to one aspect of the embodiments, the logbook interface can include multiple log entries for a selected time period (e.g., “August 1, 2019”), where the multiple log entries can comprise user-entered notes (e.g., exercise, food, medication), as well as alarm-generated log entries 2012. Further details regarding the logbook interface are described in U.S. Patent Appl. Publ. No. 2019/0183393, which is hereby incorporated by reference in its entirety for all purposes.

According to one aspect of some embodiments, the alarm-generated log entries cannot be modified by the user. In these embodiments, by contrast, other log entries (e.g., user-entered notes) can be added, edited, or deleted.

According to another aspect of some embodiments, the alarm-generated log entry 2012 can be configured such that, when pressed by the user, a logbook detail interface 2020 is displayed, as shown in FIG. 20B. In some embodiments, the logbook detail interface 2020 can include an analyte trend graph 2022 and an icon 2024 to indicate the time of the alarm event. According to some embodiments, the icon 2024 can also be graphically associated with a banner 2025 that provides further information about the analyte level or sensor reading at the time of the alarm. In addition, further alarm information 2026, comprising the type of alarm and the specific time, for example, can be displayed on the same logbook detail interface 2020. In some embodiments, other information, such as user-entered notes 2027 regarding food or exercise, can be displayed on the same logbook detail interface 2020 to provide further context to the user about the circumstances during which the alarm event occurred.

FIG. 20C is another example embodiment of a logbook detail interface 2030. As with the previous embodiment, logbook detail interface 2030 also includes comprises an icon 2032 that indicates the time of the alarm event. However, logbook detail interface 2030 includes a pop-up modal 2034 that provides further alarm information such as, e.g., the type of alarm (“Low Glucose Alarm”) and the specific time of the alarm (“1:38 PM”). In some embodiments, the pop-modal 2034 can also include other logbook entries, such as one or more user-entered logbook entries for food and/or medication (“Rapid-Acting Insulin”) to provide further context to the user about the circumstances during which the alarm event occurred.

With respect to FIGS. 20B and 20C, according to some embodiments, the logbook detail interfaces can also be configured to allow users to add and/or edit notes or other user-entered logbook entries. However, in many embodiments, alarm-generated log entries and associated alarm information cannot be modified to maintain the integrity of the data.

### Example Embodiments of Compatibility Checking for Analyte Monitoring Software Applications

Example embodiments of methods, systems, and related GUIs for onboarding and compatibility checking in an analyte monitoring software application will now be described. According to some embodiments, the various alarms and alarm features described in the previous sections can be associated with an analyte monitoring software application residing in memory of a reader device 120 (or other computing devices used in conjunction with an analyte monitoring system). Thus, in such embodiments, it would be advantageous to include certain onboarding and compatibility checking features during the setup of the analyte monitoring software application to ensure that the alarms and other features can operate as intended.

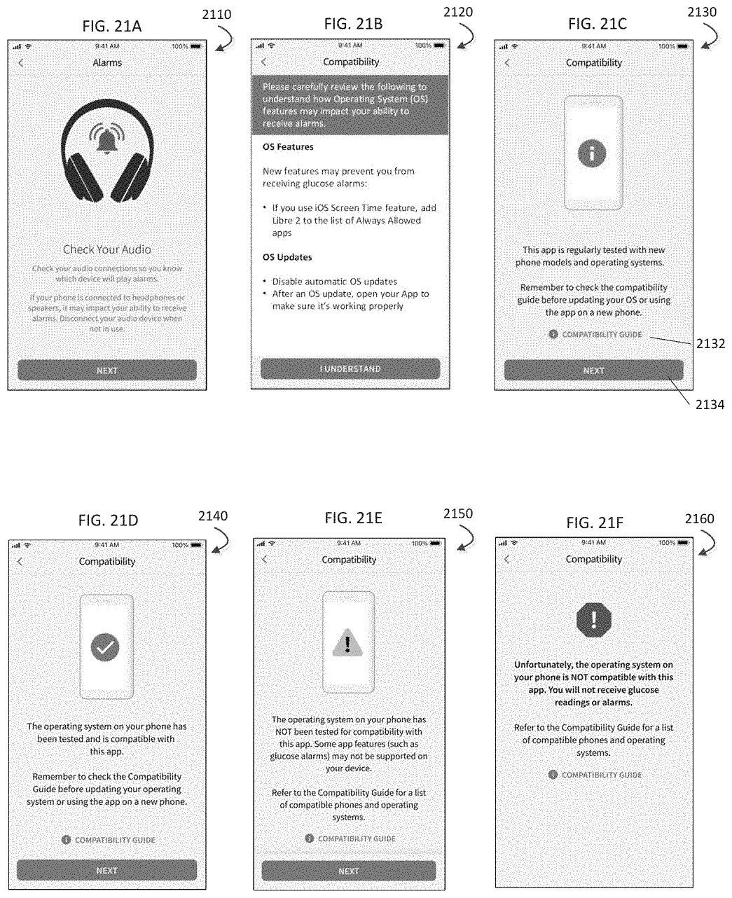

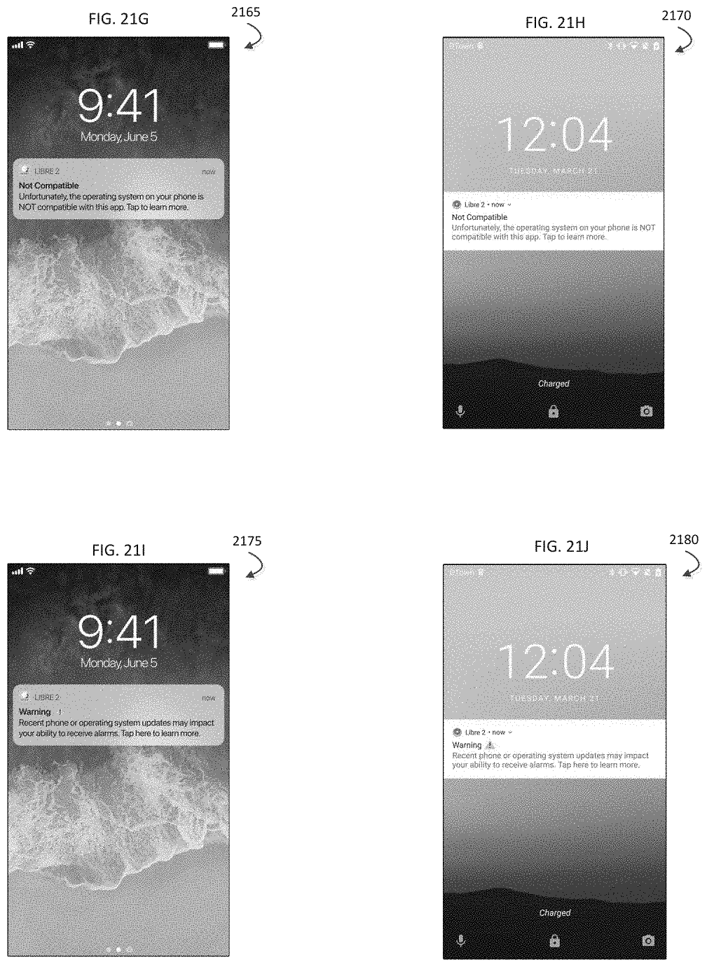

FIGS. 21A to 21F depict example embodiments of onboarding and compatibility checking interfaces for use during the setup of an analyte monitoring software application. FIG. 21A is a GUI 2110 that prompts the user to check audio connections to ensure that alarms can be properly outputted to the appropriate device. In some embodiments, GUI 2110 can also include an icon, link, or button (not shown) configured to play an audible tone when pressed for testing purposes.

FIG. 21B is a GUI 2120 depicting a compatibility interface that displays information about the operating system's features and updates that can impact the ability of the user to receive alarms. FIG. 21C is a GUI 2130 depicting a compatibility interface that includes information and an icon, link, or button 2132 to display a compatibility guide. According to one aspect of the embodiments, the compatibility guide can list all devices, operating systems, operating system types, and operating system versions that are confirmed to be compatible and/or incompatible with the analyte monitoring software application. In some embodiments, the compatibility guide can also provide information or instructions to the user about what to do if the analyte monitoring software application has been determined to be incompatible with the reader device and/or the operating system.

According to another aspect of the embodiments, GUI 2130 also includes a next button 2134 configured to perform a compatibility check. In some embodiments, the compatibility check comprises comparing the type and version of the reader device's operating system against a list, table, or database of compatible operating system types and versions stored on the reader device. In some embodiments, for example, the list, table, or database can be an encrypted data store residing on the reader device that is only accessible by the analyte monitoring software application. In other embodiments, the compatibility check can include retrieving an updated list, table, or database of compatible operating system types and versions from a remote computing system (e.g., cloud-based server). If the remote computing system is inaccessible, then the analyte monitoring software application can use a list, table, or database stored locally on the reader device. In still other embodiments, the compatibility check can comprise transmitting the type and version of the reader device's operating system to the remote computing system. Subsequently, the remote computing system sends a response back to the reader device.

According to one aspect of some embodiments, the compatibility check can result in two or more outcomes, as shown in FIGS. 21D to 21F. In many embodiments, for example, if it is determined that the operating system has been tested and is compatible with the analyte monitoring software application, then GUI 2140 is displayed, and the analyte monitoring software application is functional with a predetermined set of features enabled.

According to some embodiments, if it is determined that the operating system type or version has not been tested for compatibility with the analyte monitoring software application, then GUI 2150 is displayed along with a warning that certain features may not function correctly. In addition, in some embodiments where the operating system type or version has not been tested, certain features of the analyte monitoring software application may be disabled. In other embodiments, if the operating system type or version has not been tested, the analyte monitoring software application may still be functional with a predetermined set of features enabled.

According to many embodiments, if it is determined that the operating system type or version is not compatible with the analyte monitoring software application, then GUI 2160 is displayed, and a limited set of features are enabled for the analyte monitoring software application. For example, in some embodiments where the operating system type or version is not compatible with the analyte monitoring software application, then alarms can be disabled. In other embodiments, both alarms and sensor readings can be disabled, but the user can still review historical data or reports, if available. In still other embodiments, the entire analyte monitoring software application can be disabled.

Although the above compatibility check is described with respect to the reader device's operating system type and version, those of skill in the art will recognize that other aspects of hardware and software can be analyzed as part of the compatibility check, including but not limited to reader device model, reader device hardware componentry (e.g., minimum requirements for processor, memory, storage), other software applications installed on the same reader device, sensor control device type, sensor control device version, sensor control device model number, sensor control device firmware version, sensor control device hardware componentry, regional and/or geographical information, amongst others. This list is meant to be illustrative only, and those of skill in the art will appreciate that other factors regarding the compatibility of various software and/or hardware components of computing devices in an analyte monitoring system are fully within the scope of the present disclosure.

FIGS. 21G to 21J depict example embodiments of additional interfaces relating to compatibility checking for an analyte monitoring software application. FIGS. 21G and 21H are GUIs 2165 and 2170, respectively, each displaying a notification that the operating system of the reader device is not compatible with the analyte monitoring software application. According to some embodiments, the notification can be displayed in response to a determination of incompatibility during the onboarding or setup processes of the analyte monitoring software application, as described earlier with respect to FIGS. 21A to 21F. In other embodiments, the notification can be displayed as part of a compatibility check event that occurs after the onboarding or setup process of the analyte monitoring software application, such as, for example, as part of a compatibility check that is performed each time an update to the operating system of the reader device is detected, or each time a predetermined event occurs (e.g., user login, new sensor started, re-install of the analyte monitoring software application, etc.). In still other embodiments, the notification can be displayed as part of a compatibility check that is manually initiated by the user of the analyte monitoring software application. Similarly, FIGS. 21Iand 21J are GUIs 2175 and 2180, respectively, each displaying a notification that a recent reader (e.g., phone) or operating system update may impact the user's ability to receive alarms. According to some embodiments, the notification can be displayed in response to an alarm availability check performed during an onboarding or setup process of the analyte monitoring software application. In other embodiments, the notification can be displayed as part of an alarm availability check event that occurs each time an update to the operating system of the reader device is detected, or each a predetermined event occurs (e.g., user login, new sensor started, re-install of the analyte monitoring software application, etc.). In still other embodiments, the notification can be displayed as part of an alarm availability check that is manually initiated by the user of the analyte monitoring software application. In some embodiments, the alarm availability check can comprise a routine to check the notification permissions of the operating system, critical alert settings of the operating system, a force closure condition of the analyte monitoring software application, or any of the other related settings, configurations, and/or conditions described above in relation to alarm availability.

It will be understood by those of skill in the art that any of the GUIs (or portions thereof) described herein, are meant to be illustrative only, and that the individual elements, or any combination of elements, depicted and/or described for a particular embodiment or figure are freely combinable with any other element, or any combination of other elements, depicted and/or described with respect to any of the other embodiments.

### Example Embodiments of Interfaces for Caregiver Applications and Alarms

Example embodiments of methods and interfaces relating to caregiver applications and alarms will now be described. As an initial matter, it will be understood by those of skill in the art that the GUIs and related methods described herein comprise instructions stored in a memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the memory of a single centralized device or, in the alternative, can be distributed across multiple discrete devices in geographically dispersed locations.

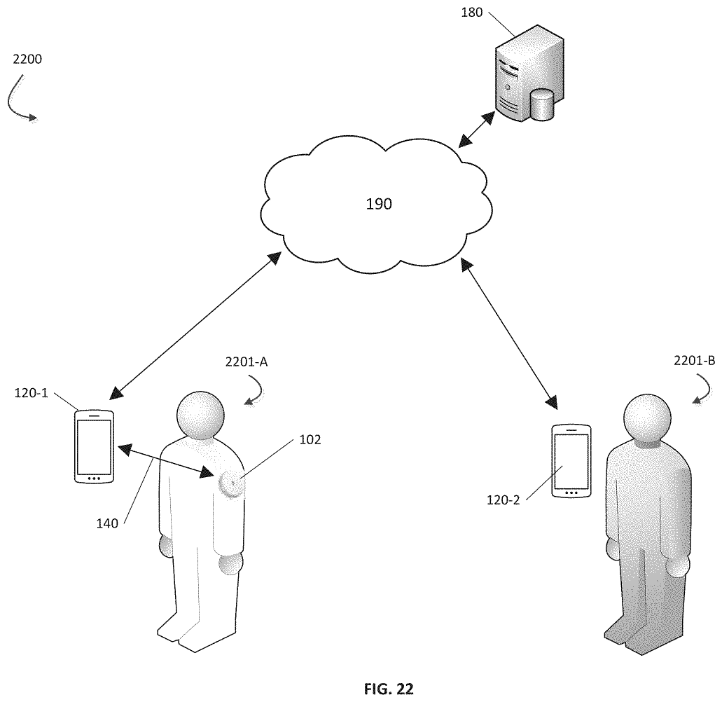

FIG. 22 is a conceptual diagram depicting an example embodiment of an analyte monitoring system 2200 capable of communicating analyte data and other information to a caregiver. According to one aspect of the embodiments, analyte monitoring system 2200 is generally similar to analyte monitoring system 100, as described with respect to FIG. 1. In particular, analyte monitoring system 2200 can comprise sensor control device 102 (worn by patient 2201-A) in wireless communication with reader device 120-1 via communication link 140. According to many of the embodiments, sensor control device 102 can include communication circuitry configured to wirelessly communicate data with reader device 120-1 via a Bluetooth communication protocol or Near Field Communication protocol. According to some embodiments, reader device 120-1 can be a smart phone, receiver device, or glucose meter. Furthermore, reader device 120-1 can be in wireless communication with trusted computer system 180 through network 190. In some embodiments, reader device 120-1 can include communication circuitry configured to wirelessly communicate data with trusted computer system 180 via an 802.11x communication protocol or a cellular communication protocol.

Referring still to FIG. 22, analyte monitoring system 2200 further comprises a second reader device 120-2 belonging to caregiver 2201-B. Second reader device 120-2 can include communication circuitry configured to wirelessly communicate data with trusted computer system 180 through network 190 via an 802.11x communication protocol or a cellular communication protocol. According to many embodiments, network 190 can be the Internet.

According to another aspect of the embodiments, sensor control device 102 includes an analyte sensor at least a portion of which is configured to be positioned in the body of patient 2201-A. Sensor control device 102 can wirelessly communicate data indicative of an analyte level of patient 2201-A to reader device 120-1, which in turn can wirelessly communicate the data to trusted computer system 180. According to another aspect of the embodiments, second reader device 120-2 includes analyte monitoring software configured to be operated by caregiver 2201-B. Furthermore, second reader device 120-2 (which can be a smart phone) is configured to receive data indicative of an analyte level of patient 2201-A, which is wirelessly communicated by trusted computer system 180 via network 190 to reader device 120-2.

In this manner, caregiver 2201-B can receive timely information regarding the analyte information of patient 2201-A. In some embodiments, for example, analyte monitoring software installed on the caregiver's reader device 120-2 can be configured to receive alarms associated with analyte levels of patient 2201-A.

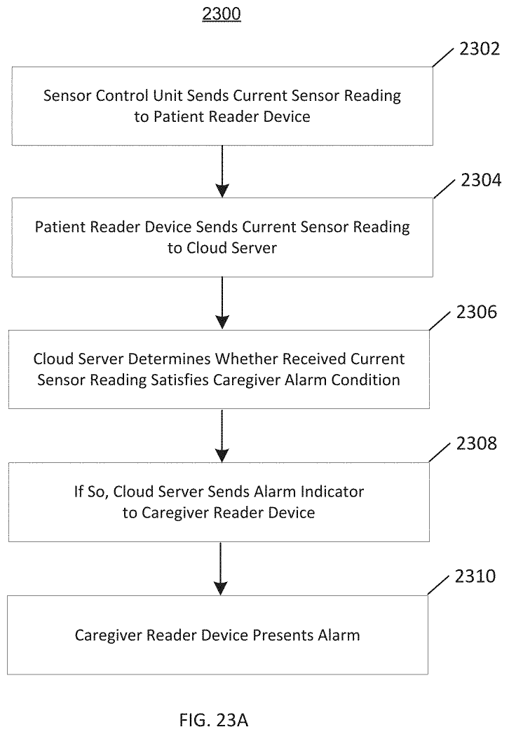

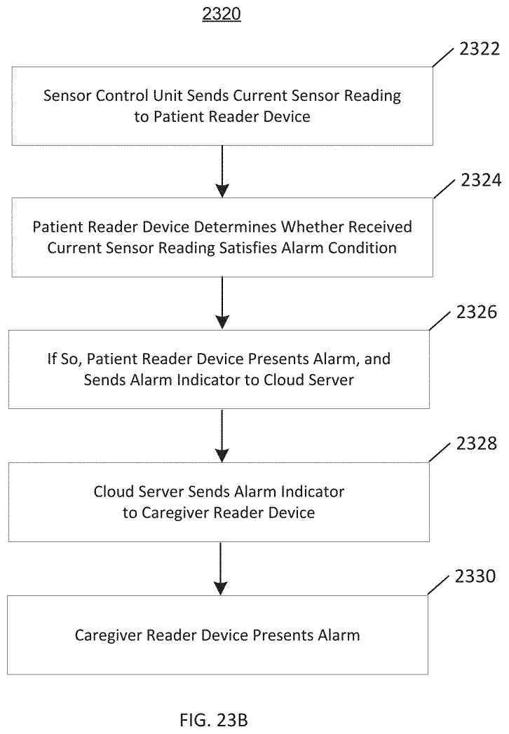

FIG. 23A is a flow diagram depicting an example embodiment of a method 2300 for providing a caregiver alarm in relation to an analyte monitoring system. At Step 2302, the sensor control unit worn by a patient communicates a current sensor reading to a patient's reader device. According to many of the embodiments, the patient's reader device can be a smart phone that includes communication circuitry configured to wirelessly communicate data with the sensor control unit according to a Bluetooth, Bluetooth Low Energy, or Near Field Communication protocol. Those of skill will recognize that other standard wireless communication protocols can be utilized and are fully within the scope of the present disclosure. At Step 2304, the patient's reader device then transmits the current sensor reading to a cloud-based server. According to many of the embodiments, the communication circuitry of the patient's reader device can be further configured to wirelessly communicate data with the cloud-based server according to an 802.11x protocol, cellular communication protocol, or any other standard wireless communication protocol.

Referring still to FIG. 23A, at Step 2306, the cloud-based server determines whether the received current sensor reading satisfies a caregiver alarm condition. According to an aspect of method 2300, the caregiver alarm condition can be configurable by the caregiver and can operate independently of the patient's own alarm conditions. In some embodiments, for example, the caregiver alarm condition can be one of: a high glucose level condition, a low glucose level condition, or a signal loss condition. In other embodiments, the caregiver alarm condition can also comprise a rate-of-change condition, a predicted glucose level condition, or a failure to dismiss an alarm condition on the patient's device. At Step 2308, if the received current sensor reading satisfies the caregiver alarm condition, then the cloud-based server then transmits an alarm indicator associated with the caregiver alarm condition to the caregiver's reader device. At Step 2310, the caregiver's reader device presents an alarm in response to receiving the alarm indicator.

FIG. 23B is a flow diagram depicting another example embodiment of a method 2320 for providing a caregiver alarm in relation to an analyte monitoring system. At Step 2322, the sensor control unit worn by a patient communicates a current sensor reading to a patient's reader device. According to many of the embodiments, the patient's reader device can be a smart phone that includes communication circuitry configured to wirelessly communicate data with the sensor control unit according to a Bluetooth, Bluetooth Low Energy, or Near Field Communication protocol. Those of skill will recognize that other standard wireless communication protocols can be utilized and are fully within the scope of the present disclosure. At Step 2324, the patient's reader device determines whether the received current sensor reading satisfies an alarm condition. In some embodiments, for example, the alarm condition can be one of: a high glucose condition, a low glucose condition, a signal loss condition, a rate-of-change condition, or a predicted glucose level condition, among others.

Referring still to FIG. 23B, at Step 2326, if an alarm condition is satisfied, then the patient's reader device presents an alarm to the patient and, furthermore, transmits an alarm indicator associated with the determined alarm condition to the cloud-based server. At Step 2328, the cloud-based server then transmits the received alarm indicator associated with the determined alarm condition to the caregiver's reader device. At Step 2330, the caregiver's reader device presents an alarm based on the received alarm indicator associated with the determined alarm condition.

According to an aspect of method 2320, the analyte monitoring system is configured such that the caregiver is only presented with alarms configured by the patient. In other words, according to the embodiment of method 2320, the alarm indicators received by the caregiver merely “pass through” the cloud-based server without any independent evaluation performed by the either the cloud-based server or the caregiver's reader device.

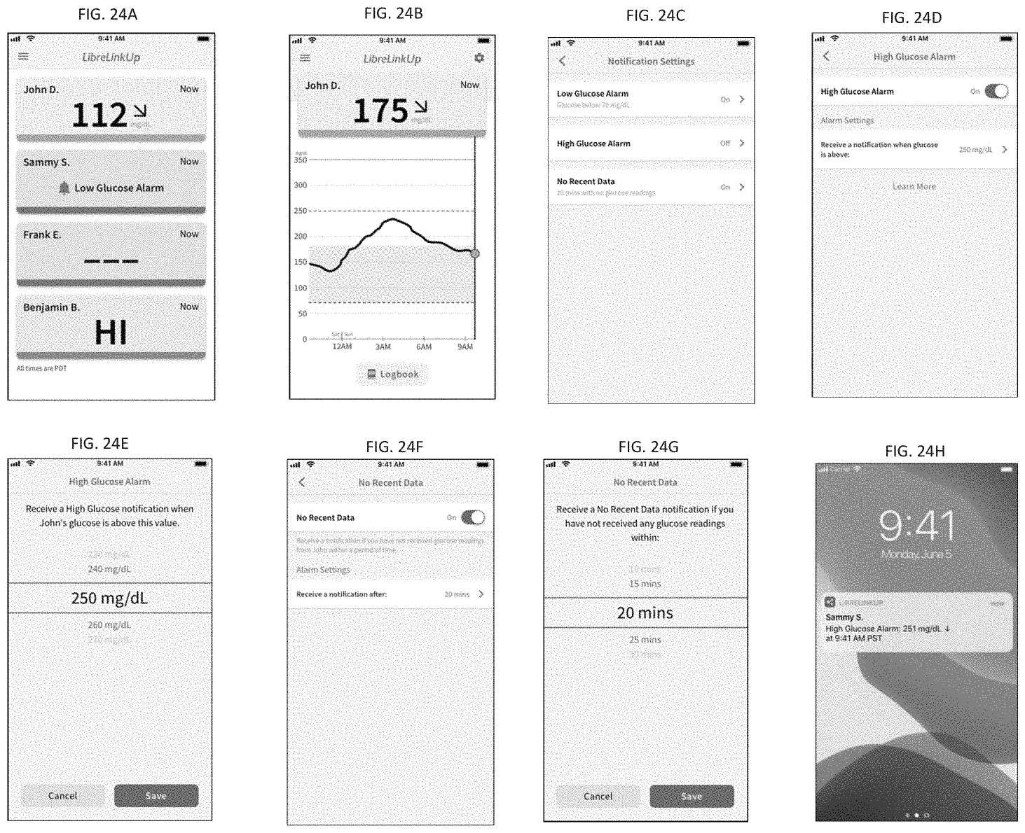

FIGS. 24A to 24H depict various GUIs and alarm interfaces of an analyte monitoring software application configured to operate on a caregiver's reader device. FIG. 24A is a GUI depicting a home interface with multiple connections, each depicted as a profile card. FIG. 24B is a GUI depicting an analyte graph for a specific connection. FIG. 24C is a GUI depicting an alarm configuration interface for toggling on/off a low glucose alarm, high glucose alarm, and a no recent data alarm. FIG. 24D is a GUI depicting another alarm configuration interface for a high glucose alarm, in which the interface indicates the current value for a high glucose alarm threshold. FIG. 24E is a GUI depicting still another alarm configuration interface for a high glucose alarm, in which the interface includes a user configurable setting for a high glucose alarm threshold. FIG. 24F is a GUI depicting an alarm configuration interface for a no recent data alarm, in which the interface indicates the current value for a notification time period after which the no recent data alarm is triggered. FIG. 24G is another GUI depicting an alarm configuration interface for a no recent data alarm, in which the interface includes a user configurable setting for a notification time period after which the no recent data alarm is triggered. FIG. 24H depicts an alarm interface for a high glucose alarm.

Example Embodiments for Displaying Non-Medical Data from a Sensor Control Device

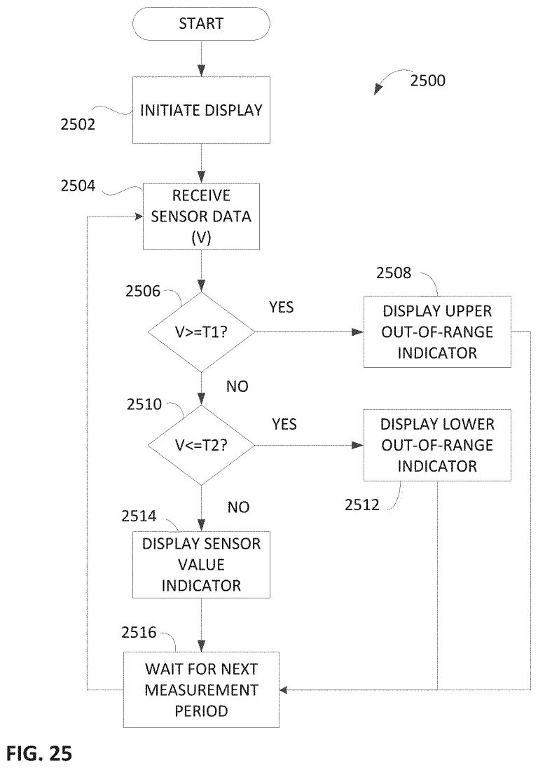

Example embodiments of methods, apparatuses, and systems, including graphical user interfaces, for the display of non-medical data from a sensor control device will now be described. FIG. 25 is a flowchart illustrating a process 2500 for an electronic interface of a computing device (e.g., a reader device as described herein) that displays non-medical data from a sensor control device 102. To initiate display 2502, at least one processor of the computing device may receive sensor data 2504 collected by a sensor control device 102, for example, data indicating a glucose level. The sensor control device may provide the sensor data at periodic intervals, for example, once per second, once per 15, 30, or 45 seconds, once per minute, or once per 2, 3, 4 or 5 minutes, using any suitable wireless or wired connection. In an alternative, or in addition, the sensor control device may provide the sensor data in response to a detected event, for example, a number of user heartbeats, a number of user breaths, or in response to user input to a reader device indicating a data request. If the sensor control device and reader device are not made for the same non-medical application, the reader device will be unable to receive data from the sensor control device, and the process 2500 will terminate, optionally providing an error message to the user (not shown).

At 2506, the at least one processor may determine whether each measurement value of the sensor data received at block 2504 is greater than or equal to a predetermined upper threshold, which the data must not be to satisfy at least one condition for display as non-medical information. For example, a measurement value of the sensor data 2504 exceeding the predetermined upper threshold 2506 may indicate of a medical pathology or condition such that communicating the value is inappropriate for non-medical use. If so, the processor may, without indicating the sensor data value, provide an upper out-of-range indicator 2508 to the user interface for display.

If after determining that the sensor data does not exceed an upper threshold at 2506, the at least one processor may determine, at 2510, whether the sensor value is less than or equal to a predetermined lower threshold, which the data must not be to satisfy at least one condition for display as non-medical information. For example, a measurement value of the sensor data 2504 being less than the predetermined lower threshold 2510 may indicate of a medical pathology or condition such that communicating the value is inappropriate for non-medical use. If so, the processor may, without indicating the sensor data value, provide a lower out-of-range indicator 2512 to the user interface for display. If not, the processor may provide the measurement value to the user interface for display, at 2514.

At 2516, after the sensor data or an out of range indicator has been provided for display by the user interface, the processor waits for the next measurement value, which may be provided periodically or in response to an event as noted above. The processor may continue the process 2500 until terminated by the user, or in response to expiration of a time period or occurrence of any other suitable terminating event. Thus, using the process 2500, the processor may provide a signal to display a sensor value indicator on a reader device only when a measurement value falls within a range of values that satisfy at least one condition for display as non-medical information.

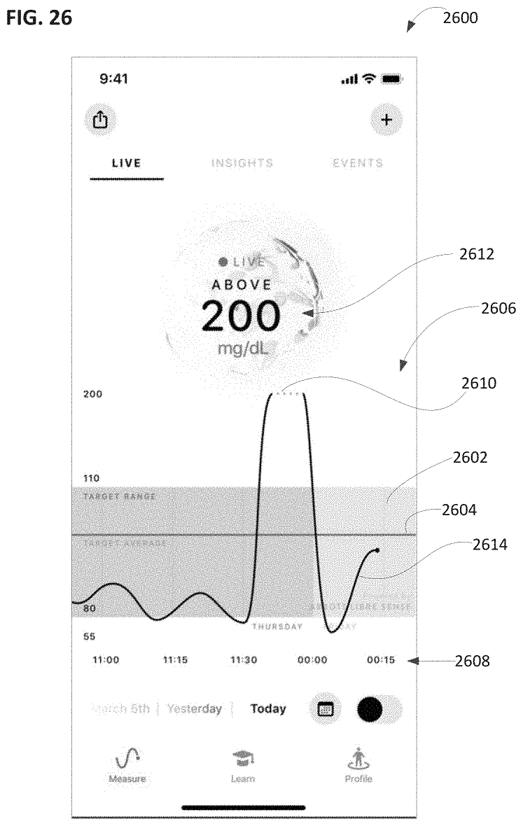

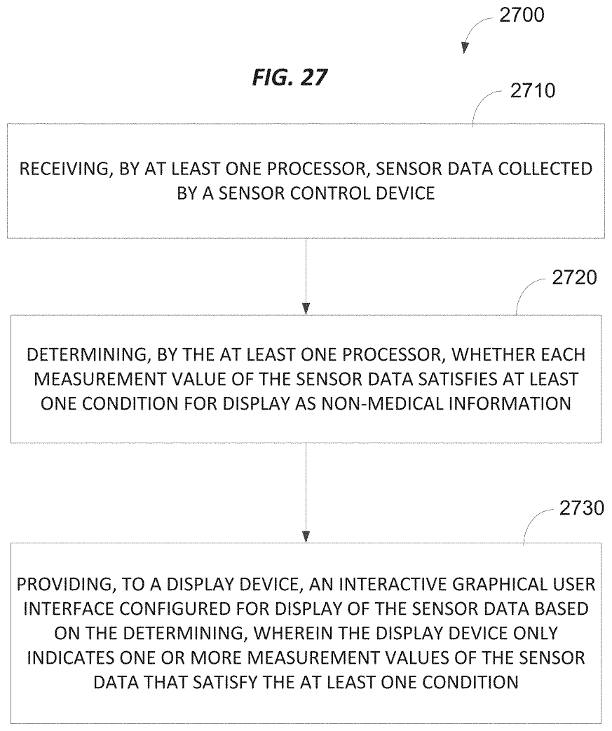

FIG. 26 is a screenshot illustrating an example display window 2600 output by an interactive graphical user interface as may be provided using the process 2500 or method 2700 (FIG. 27). The display window 2600 may include a graph 2606 indicating the analyte measurement values provided by sensor data over time 2608 and a graphical object 2612 providing a display text of a current or most recent measurement value. Additionally, the graph 2606 may include an indicator of a target average 2604 for measured analyte (e.g., glucose) values and a range 2602 that indicates the limit of non-medical values within which display of measurement values is exclusively allowed. For example, in the illustrated embodiment for monitoring glucose levels for sports use, the graph 2606 includes a range 2602 between an upper threshold measurement value of 200 mg/dL and a lower threshold measurement value of 55 mg/dL. The threshold values are merely illustrative, and other values may also be suitable for defining a limit of non-medical information, depending on the analyte and intended application.

A processor of a reader device may, for measurement values within the glucose level range 2602, cause the graph 2606 to indicate the most recent and past measurement values as segments or points of a plotted line 2614. Conversely, for measurement values out of range, the processor may cause the graph 2606 to indicate an out-of-range condition, for example, a dashed horizontal line 2610 indicating that data exceeds the glucose threshold limits. In addition, if the user's glucose level is out of range, the processor may cause the graphical object 2612 to display and out-of-range message, for example that the glucose level is “above 200 mg/dL” or “below 55 mg/dL.”

In summary of the foregoing, and by way of additional example, FIG. 27 shows operations of a method 2700 for performing the process for an electronic interface that displays non-medical data from a sensor control device 102. At 2710, the method for an electronic interface includes receiving, by at least one processor, sensor data collected by a sensor control device 102. In one embodiment of the present invention, the electronic interface receives performance-affecting analytes, for example blood glucose and/or lactate, before or during athletic training and competition so as to enable the user to track and understand glucose levels in order to maintain peak performance. At 2720, the method further includes determining, by the at least one processor, whether each measurement value of the sensor data satisfies at least one condition for display as non-medical information. For example, for an application providing glucose monitoring for sports use, a non-medical glucose level range may be defined between specified levels, such as 55 mg/dL and 200 mg/dL.

At 2730, the method 2700 may include providing, to a display device, an interactive graphical user interface configured for display of the sensor data based on the determining, wherein the display device only indicates one or more measurement values of the sensor data that satisfy the at least one condition. For example, as shown in FIG. 26 for an application measuring blood glucose for athletes, a non-medical glucose level range between 55 mg/dL and 200 mg/dL may be defined. If a user's analyte measurement value falls within this range, it satisfies at least one condition for display as non-medical information. Conversely, a user's analyte measurement value may be treated as indicative of a medical pathology or condition if it exceeds the bounds of the glucose range. Thus, the at least one processor may determine out-of-range data fails to satisfy the defined condition for non-medical information and prevent display of out-of-range data by the graphical user interface.

A processor and memory holding instructions for performing operations of the method 2700 may be, or may include, a means for performing the operations illustrated by the blocks 2710, 2720 and 2730. Said means may include more detailed algorithms for performing said operations. For example, a more detailed algorithm for performing the operation 2710 may include initiating a wireless session with a sensor control device using a wireless protocol, for example, Bluetooth Low Energy (BLE), receiving a wireless signal from the sensor control device, generating digital data in a transport layer based on the wireless signal, and providing the digital data to an application layer. For further examples, a more detailed algorithm for performing the operation 2720 may include the operations 406 and 410 described in connection with FIG. 4. A more detailed algorithm for performing the operation 2730 may include the operations 2508, 2512 and 2514 described in connection with FIG. 25, wherein these operations are conditioned on the upstream branching operations 2506, 2510 and wherein the processor formats data for generating the display by a targeted display device.

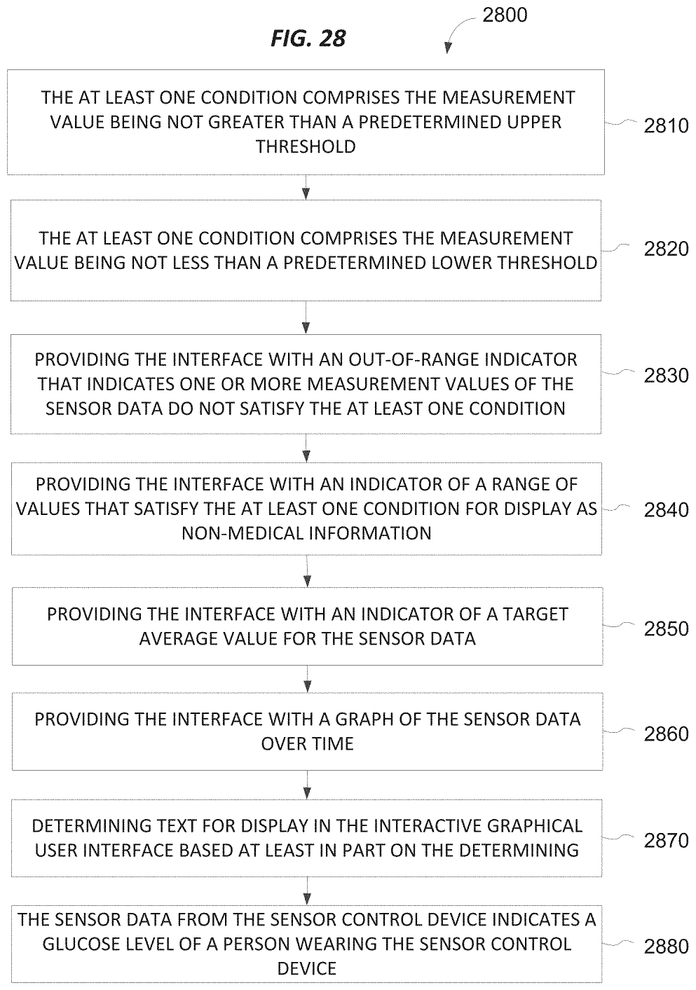

FIG. 28 shows further, optional operations or aspects 2800 that may be included in the method 2700 by at least one processor of a reader device. Said operations or aspects 2800 may be included in the method 2700 in any operative order. Inclusion or omission of any upstream one of the operations or aspects 2800 does not necessarily require that any downstream operation be likewise included or omitted.

In an aspect, the determining operation 2720 of the method 2700 may further include, at 2810, applying the at least one condition that includes the measurement value being not greater than a predetermined upper threshold. For example, when the electronic interface is collecting glucose analyte data for sports use, the predetermined upper threshold may be 200 mg/dL. Likewise, at 2820, the determining operation 2720 of the method 2700 may further include applying the at least one condition that includes the measurement value being not less than a predetermined lower threshold. For example, wherein glucose levels are measured for sports use and non-medical purpose, the predetermined lower threshold 2510 value for glucose may be 55 mg/dL. At 2830, the determining operation 2720 of the method 2700 may further include providing the interface with an out-of-range indicator that indicates one or more measurement values of the sensor data do not satisfy the at least one condition. In an aspect, the out-of-range indicator indicates ones of the measurement values are out of range without indicating values that are out of the range, for example as shown by the dotted line 2610 of FIG. 26.

In another aspect, the providing operation 2730 of the method 2700 may further include, at 2840, providing the interface with an indicator of a range of values that satisfy the at least one condition for display as non-medical information. For example, at least one condition for display as non-medical information may be satisfied if the user's glucose level is between the predetermined upper and lower threshold range of 55 mg/dL and 200 mg/dL, and the processor may accordingly cause display of a range indicator 2602 as shown in FIG. 26. In addition, the providing operation 2730 may further include, at 2850, providing the interface with an indicator of a target average value for the sensor data, for example as shown at 2604 of FIG. 26. Optionally, the target average value may be set by the user depending on activity. For example, the processor may provide a menu of target value options for different activity and set the target value based on the user-selected activity. In a related aspect, the providing operation 2730 may further include, at 2860, providing the interface with a graph of the sensor data over time, for example, as shown at 2606 and 2614 of FIG. 26.

In another aspect, the method 2700 may further include, at 2870, the at least one processor determining text for display in the interactive graphical user interface based at least in part on the determining of whether the at least one condition for display as non-medical information is satisfied. For example, the processor may limit user interface output to displaying glucose levels falling within the range of the predetermined upper and lower threshold values, as shown at 2612 of FIG. 26. When the user's glucose level exceeds the upper threshold, for example, the graphical object 2612 displays a message that glucose levels are “above 200 mg/dL.” Likewise, the processor may cause the object 2612 to display “below 55 mg/dL” for measurement values that fall below the lower threshold.

In an aspect as noted above, at 2880, the sensor data from the sensor control device used in the method 2700 may indicate a glucose level of a person wearing the sensor control device. However, the method is not limited to glucose as an analyte and may similarly control use and display of any useful analyte for non-medical use, such as, for example, blood oxygen level and/or lactate. In another aspect, the sensor control device and reader device may be configured so that only devices configured for a compatible non-medical application can access data from the sensor control device. Thus, for example, a medical sensor control device cannot provide data to a non-medical reader device, and a non-medical sensor control device cannot provide data to a medical reader device.

In some embodiments, the non-medical data from the sensor control device can be outputted to a display of a wearable device, such as a fitness tracker or a smart watch. In some embodiments, the non-medical data can be displayed on the wearable device and the reader device at the same time. In other embodiments, the user can select the device to display the non-medical device.

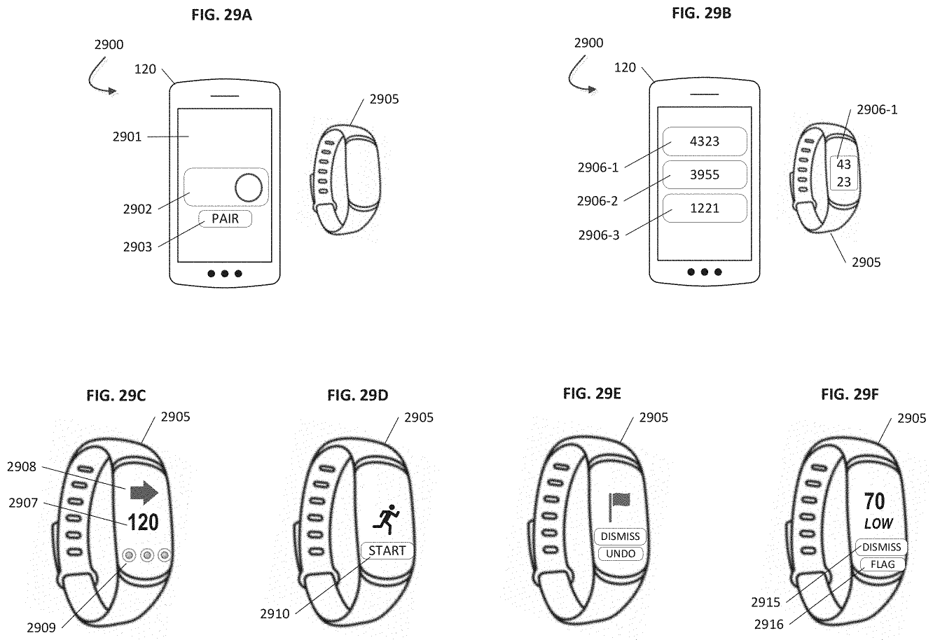

FIGS. 29A to 29G depict various example embodiments of user interfaces for displaying non-medical data on a wearable device. FIG. 29A depicts a system 2900 for displaying non-medical data from a sensor control device, wherein system 2900 includes a reader device 120 and a wearable device 2905. According to one aspect of the embodiments, reader device 120 can include software stored in memory, which can be part of the same software described with respect to FIG. 26. In some embodiments, software can be configured to display user interface 2901, which can comprise a sensor information section 2902 and a pairing button 2903. Sensor information section 2902 can include information about the sensor control device (not shown), including but not limited to, an image of the sensor control device, model name, model number, serial number, connection status, and remaining days of use (e.g., amount of time until sensor expires). Pairing button 2903 can be configured to initiate a pairing sequence.

FIG. 29B depicts system 2900 for displaying non-medical data from a sensor control device on a wearable device, in which a pairing sequence has been initiated on reader device 120. According to some embodiments, multiple selectable pairing codes, 2906-1, 2906-2, and 2906-3, are outputted to display of reader device 120. In addition, a single pairing code 2906-1 is outputted to the display of wearable device 2905. Accordingly, the user can select the correct pairing code 2906-1 on reader device 120 that matches the pairing code displayed on wearable device 2905. Once the correct pairing code 2906-1 is selected, then reader device 120 can be paired with the wearable device 2905. Subsequently, according to some embodiments, reader device 120 can transmit sensor context information to wearable device 2905, such that wearable device 2905 is then able to communicate directly with sensor control device (not shown). According to another aspect of some embodiments, before a communication link is established between wearable device 2905 and sensor control device, an existing communication link between reader device 120 and sensor control device (not shown) is deactivated. Further details regarding the establishment of multiple communication links between multiple devices and a sensor control device are described in International Publ. No. WO 2021/087013A1, which is hereby incorporated by reference in its entirety for all purposes.

FIGS. 29C to 29F depict interfaces on wearable device 2905 once a communication link has been established between wearable device 2905 and sensor control device (not shown). FIG. 29C, for example, depicts a real-time glucose level reading 2907 received from sensor control device along with a trend indicator 2908 (e.g., an arrow to indicate whether the glucose level is rising, falling, or staying the same). According to some embodiments, wearable device 2905 can also be configured to display a graph (not shown). According to one aspect of the embodiments, the graph can comprise a first axis indicative of a time unit and a second axis indicative of an analyte concentration (e.g., glucose concentration). According to another aspect of the embodiments, the graph can reflect analyte level measurements over time, shown as a plotted line, discrete data points, or both. In some embodiments, the graph can be displayed instead of real-time glucose level reading 2907 and trend indicator 2908. In other embodiments, the graph can be displayed on the same screen as either or both of real-time glucose level reading 2905 and/or trend indicator 2908. Furthermore, in some embodiments, one or more status indicators 2909 can also be displayed on wearable device 2905. The status indicators 2909 can comprise lights, for example, to indicate an active connection/pairing with a sensor control device, an active connection/pairing with an app (e.g., on reader device 120), an ongoing event, a battery indicator, or a sensor life remaining indicator. Although FIG. 29C depicts the one or more indicators 2909 as a plurality of lights, those of skill in the art will recognize that other types of indicators (e.g., textual, numeric (as a percentage or a score), graphical) can also be utilized, and are fully within the scope of the present disclosure.

FIG. 29D depicts an events interface on wearable device 2905. According to some embodiments, a user can indicate that an event is occurring by selecting the event start button 2910 on the events interface. An event can be one or more of exercise, sleep, or a meal, to name only a few. For example, a user may choose to start an event to track their glucose during a workout or a race. During an event, a user can flag their glucose data via a flag interface, as shown in FIG. 29. According to one aspect of the embodiments, a flag will bookmark the user's glucose data at a particular point in time during events of interest. FIG. 29F is an alarms interface on wearable device 2905. According to some embodiments, alarms can notify the user when the glucose level exceeds a predetermined threshold (e.g., low glucose threshold, high glucose threshold, etc.). In some embodiments, the predetermined threshold can also be a rate-of-change threshold or a signal loss threshold. The alarm can be a visual indicator, such as using a red color for the numeric or textual display, a flashing light, a flashing text, or the activation of a backlight. According to some embodiments, the alarm can also be an audible or vibratory indicator. The alarm can be dismissed (e.g., via a dismiss button 2915) or flagged (e.g., via a flag button 2916) for later review.

Although many of the embodiments described herein relate to glucose monitoring, those of skill in the art will appreciate that these same embodiments can be implemented for purposes of monitoring other analytes, such as, for example, lactate and ketones. By way of illustration only, for example, with respect to process 2500 (FIG. 25), display window 2600 (FIG. 26), and/or methods 2700 and 2800 (FIGS. 27 and 28), it will be understood by those of skill in the art that any or all of the aforementioned embodiments can be implemented for purposes of monitoring other analytes besides glucose, such as, for example, lactate or ketones. Similarly, embodiments of alarms, such as those described with respect to FIG. 29F, can be based on a lactate threshold (e.g., low lactate threshold, high lactate threshold) or a ketone threshold.

Furthermore, those of skill in the art will appreciate that the embodiments described herein are not limited to the monitoring one analyte at a time, although each embodiment described herein is capable of doing so. For example, according to some embodiments, a single sensor control device can include within its housing, for example, an analyte sensor capable of sensing an in vivo glucose level and an in vivo lactate level in a bodily fluid of the user. Likewise, any and all of the aforementioned embodiments of processes, display windows, methods, and/or alarms can be configured for purposes of monitoring multiple analytes at once (e.g., glucose and lactate, glucose and ketone, etc.).

In summary, improved digital interfaces, graphical user interfaces, and alarms for analyte monitoring systems are provided. For example, disclosed herein are various embodiments of methods, systems, and interfaces for signal loss condition determination, Time-in-Ranges interfaces, GMI metrics, urgent low glucose alarms, alarm suppression features, alarm setup interfaces, and alarm unavailability detection features. In addition, various embodiments of interfaces for alarm logging and compatibility checking of an analyte monitoring software application are described. Also, various embodiments of interface enhancements are described, including an enhanced visibility mode, a voice accessibility mode, additional interfaces relating to user privacy, as well as caregiver alarms, among other embodiments.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. An analyte monitoring system, comprising:
a sensor control device comprising an analyte sensor, wherein at least a portion of the analyte sensor is configured to be in fluid contact with a bodily fluid of a subject; and
a reader device, comprising:
wireless communication circuitry configured to receive a current sensor reading from the sensor control device; and
one or more processors coupled to a memory, the memory storing instructions that, when executed by the one or more processors, cause the one or more processors to:
determine a time elapsed since the current sensor reading was received,
determine whether the time elapsed exceeds a signal loss indicator threshold, and
in response to a determination that the time elapsed exceeds the signal loss indicator threshold, generate a signal loss indicator.

2. The analyte monitoring system of claim 1, wherein the signal loss indicator threshold is five minutes.

3. The analyte monitoring system of claim 1, wherein the reader device is a smart phone.

4. The analyte monitoring system of claim 1, wherein the instructions, when executed by the one or more processors, further cause the one or more processors to output a signal loss message to a display of the reader device.

5. An analyte monitoring system, comprising:
a sensor control device comprising an analyte sensor, wherein at least a portion of the analyte sensor is configured to be in fluid contact with a bodily fluid of a subject; and
a reader device, comprising:
wireless communication circuitry configured to receive a current sensor reading from the sensor control device; and
one or more processors coupled to a memory, the memory storing instructions that, when executed by the one or more processors, cause the one or more processors to:
determine a time elapsed since the current sensor reading was received,
determine whether the time elapsed exceeds a signal loss indicator threshold,
in response to a determination that the time elapsed exceeds the signal loss indicator threshold, generate a signal loss indicator,
determine whether the current sensor reading is invalid, and
in response to a determination that the current sensor reading is invalid, generating an invalid sensor reading indicator.

6. The analyte monitoring system of claim 5, wherein the invalid sensor reading indicator is a sensor error indicator or a temperature error indicator.

7. The analyte monitoring system of claim 6, wherein the temperature error indicator is based on a temperature measurement obtained by the sensor control device.

8. The analyte monitoring system of claim 6, wherein the sensor error indicator is based on an early signal attenuation condition detected by the sensor control device.

9. The analyte monitoring system of claim 5, wherein the instructions, when executed by the one or more processors, further cause the one or more processors to display a valid current sensor reading in response to a determination that the current sensor reading is not invalid.

10. An analyte monitoring system, comprising:
a sensor control device comprising an analyte sensor, wherein at least a portion of the analyte sensor is configured to be in fluid contact with a bodily fluid of a subject; and
a reader device, comprising:
wireless communication circuitry configured to receive a current sensor reading from the sensor control device; and
one or more processors coupled to a memory, the memory storing instructions that, when executed by the one or more processors, cause the one or more processors to:
determine whether the current sensor reading is valid,
in response to a determination that the current sensor reading is not valid, generate an invalid current sensor reading indicator and determine a time elapsed since a last valid current sensor reading,
determine whether the time elapsed since the last valid current sensor reading exceeds a recent valid reading alarm threshold,
in response to a determination that the time elapsed since the last valid current sensor reading exceeds the recent valid reading alarm threshold, generate a no recent valid reading alarm.

11. The analyte monitoring system of claim 10, wherein the recent valid reading alarm threshold is twenty minutes.

12. The analyte monitoring system of claim 10, wherein the reader device is a smart phone.

13. The analyte monitoring system of claim 10, wherein the no recent valid reading alarm comprises a temporary notification configured to disappear after a predetermined amount of time without user interaction.

14. The analyte monitoring system of claim 10, wherein the no recent valid reading alarm comprises an alarm interface configured to persist on a display of the reader device until the subject acknowledges or dismisses the alarm interface.

15. The analyte monitoring system of claim 10, wherein the instructions to determine whether the current sensor reading is valid comprises an instruction to determine whether a sensor error condition is present followed by an instruction to determine whether a temperature error condition is present.

16. The analyte monitoring system of claim 10, wherein the instructions to determine whether the current sensor reading is valid is preceded by an instruction to determine whether a signal loss condition is present.

17. The analyte monitoring system of claim 10, wherein the instructions, when executed by the one or more processors, further cause the one or more processors to determine whether a high glucose level condition or a low glucose level condition is present.

18. The analyte monitoring system of claim 17, wherein the instructions, when executed by the one or more processors, further cause the one or more processors to generate an analyte condition indicator in response to a determination that a high glucose level condition or a low glucose level condition is present.

19. The analyte monitoring system of claim 10, wherein the instructions, when executed by the one or more processors, further cause the one or more processors to display the current sensor reading in response to a determination that the current sensor reading is valid.

20-278. (canceled)
